SEQUENCE LISTING (45 sequences)

SEQ ID NO: 1 - protein, 5 aa, Artificial Sequence
  Synthetic Construct
DYGMV

SEQ ID NO: 2 - protein, 17 aa, Artificial Sequence
  Synthetic Construct
FISSGSSTVYYADTMKG

SEQ ID NO: 3 - protein, 10 aa, Artificial Sequence
  Synthetic Construct
RNYDDWYFDV

SEQ ID NO: 4 - protein, 10 aa, Artificial Sequence
  Synthetic Construct
SASSSVTYMY

SEQ ID NO: 5 - protein, 7 aa, Artificial Sequence
  Synthetic Construct
RTSDLAS

SEQ ID NO: 6 - protein, 9 aa, Artificial Sequence
  Synthetic Construct
QHYHSYPLT

SEQ ID NO: 7 - protein, 119 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSS

SEQ ID NO: 8 - protein, 106 aa, Artificial Sequence
  Synthetic Construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIK

SEQ ID NO: 9 - protein, 448 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 213 aa, Artificial Sequence
  Synthetic Construct
DIQMTQSPSSLSASVGDRVTITCSASSSVTYMYWYQQKPGKSPKPWIYRTSDLASGVPSRFSGSGSGTDFTLTISSLQPEDFATYYCQHYHSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 445 aa, Artificial Sequence
  Synthetic Construct
EVQLVESGGGLVQPGGSLRLSCAASGFTFSDYGMVWVRQAPGKGLEWVAFISSGSSTVYYADTMKGRFTISRDNSKNTLYLQMNSLRAEDTAVYYCTRRNYDDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 5 aa, Artificial Sequence
  Synthetic Construct
GYAIT

SEQ ID NO: 13 - protein, 16 aa, Artificial Sequence
  Synthetic Construct
GISSAATTFYSSWAKS

SEQ ID NO: 14 - protein, 14 aa, Artificial Sequence
  Synthetic Construct
DPRGYGAALDRLDL

SEQ ID NO: 15 - protein, 12 aa, Artificial Sequence
  Synthetic Construct
QSIKSVYNNRLG

SEQ ID NO: 16 - protein, 7 aa, Artificial Sequence
  Synthetic Construct
ETSILTS

SEQ ID NO: 17 - protein, 11 aa, Artificial Sequence
  Synthetic Construct
AGGFDRSGDTT

SEQ ID NO: 18 - protein, 122 aa, Artificial Sequence
  Synthetic Construct
EVQLVESGPGLVKPSETLSLTCTVSRFSLIGYAITWIRQPPGKGLEWIGGISSAATTFYSSWAKSRVTISRDTSKNQVSLKLSSVTAADTAVYYCARDPRGYGAALDRLDLWGQGTLVTVSS

SEQ ID NO: 19 - protein, 110 aa, Artificial Sequence
  Synthetic Construct
DIQMTQSPSSLSASVGDRVTITCQSIKSVYNNRLGWYQQKPGKAPKLLIYETSILTSGVPSRFSGSGSGTDFTLTISSLQPEDFATYYCAGGFDRSGDTTFGQGTKVEIK

SEQ ID NO: 20 - protein, 451 aa, Artificial Sequence
  Synthetic Construct
EVQLVESGPGLVKPSETLSLTCTVSRFSLIGYAITWIRQPPGKGLEWIGGISSAATTFYSSWAKSRVTISRDTSKNQVSLKLSSVTAADTAVYYCARDPRGYGAALDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 217 aa, Artificial Sequence
  Synthetic Construct
DIQMTQSPSSLSASVGDRVTITCQSIKSVYNNRLGWYQQKPGKAPKLLIYETSILTSGVPSRFSGSGSGTDFTLTISSLQPEDFATYYCAGGFDRSGDTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 448 aa, Artificial Sequence
  Synthetic Construct
EVQLVESGPGLVKPSETLSLTCTVSRFSLIGYAITWIRQPPGKGLEWIGGISSAATTFYSSWAKSRVTISRDTSKNQVSLKLSSVTAADTAVYYCARDPRGYGAALDRLDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 24 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 25 - protein, 233 aa, Homo sapiens
MLNLLLLALPVLASRAYAAPAPGQALQRVGIVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQVATAPHTFPAPS

SEQ ID NO: 26 - protein, 245 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 27 - protein, 256 aa, Homo sapiens
IVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVKVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

SEQ ID NO: 28 - protein, 256 aa, Homo sapiens
IVGGQEAPRSKWPWQVSLRVRDRYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKPGNSDYKDDDDK

SEQ ID NO: 29 - protein, 256 aa, Homo sapiens
IVGGQEAPRSKWPWQVSLRVRDRYWMHFCGGSLIHPQWVLTAAHCLGPDVKDLAALRVQLREQHLYYQDQLLPVSRIIVHPQFYIIQTGADIALLELEEPVNISSRVHTVMLPPASETFPPGMPCWVTGWGDVDNDEPLSPPFPLKQVKVPIMENHICDAKYHLGAYTGDDVRIIRDDMLCAGNSQRDSCKGDSGGPLVCKVNGTWLQAGVVSWDEGCAQPNRPGIYTRVTYYLDWIHHYVPKKPGNSDYKDDDDK

SEQ ID NO: 30 - protein, 6 aa, Artificial Sequence
  Synthetic Construct
FSLLRY

SEQ ID NO: 31 - DNA, 20 bp, Artificial Sequence
  Synthetic Construct
ctggtgtgcaaggtgaatgg

SEQ ID NO: 32 - DNA, 20 bp, Artificial Sequence
  Synthetic Construct
aggtccagcactcaggagga

SEQ ID NO: 33 - DNA, 20 bp, Artificial Sequence
  Synthetic Construct
gcaggtgagcctgagagtcc

SEQ ID NO: 34 - DNA, 20 bp, Artificial Sequence
  Synthetic Construct
gggaccttcacctgcttcag

SEQ ID NO: 35 - DNA, 18 bp, Artificial Sequence
  Synthetic Construct
cagccagtgacccagcac

SEQ ID NO: 36 - DNA, 51 bp, Homo sapiens
  misc_feature (26)..(26): n is g or a
ctgcaggcgggcgtggtcagctgggncgagggctgtgcccagcccaaccgg

SEQ ID NO: 37 - DNA, 42 bp, Homo sapiens
cacacggtcaccctgccccctgcctcagagaccttccccccc

SEQ ID NO: 38 - protein, 121 aa, Artificial Sequence [sequence omitted]

SEQ ID NO: 39 - protein, 111 aa, Artificial Sequence [sequence omitted]

SEQ ID NO: 40 - protein, 5 aa, Artificial Sequence
  Synthetic Construct
GYSWN

SEQ ID NO: 41 - protein, 16 aa, Artificial Sequence
  Synthetic Construct
SITYDGSTNYNPSVKG

SEQ ID NO: 42 - protein, 12 aa, Artificial Sequence
  Synthetic Construct
GSHYFGHWHFAV

SEQ ID NO: 43 - protein, 15 aa, Artificial Sequence
  Synthetic Construct
RASQSVDYDGDSYMN

SEQ ID NO: 44 - protein, 7 aa, Artificial Sequence
  Synthetic Construct
AASYLES

SEQ ID NO: 45 - protein, 9 aa, Artificial Sequence
  Synthetic Construct
QQSHEDPYT

(19) United States
(12) Patent Application Publication
(10) Pub. No.: US 2020/0377953 A1
(43) Pub. Date: Dec. 3, 2020

(54) THERAPEUTIC AND DIAGNOSTIC METHODS FOR MAST CELL-MEDIATED INFLAMMATORY DISEASES

(71) Applicant: Genentech, Inc., South San Francisco, CA (US)

(72) Inventors: David F. CHOY, Fairfield, CA (US); Tracy Lyn STATON, Stanford, CA (US); Brian Louis YASPAN, Pacifica, CA (US)

(21) Appl. No.: 16/987,958

(22) Filed: Aug. 7, 2020

Related U.S. Application Data
(63) Continuation of application No. PCT/US2019/017320, filed on Feb. 8, 2019.
(60) Provisional application No. 62/628,564, filed on Feb. 9, 2018.

(51) Int. Cl. C12Q 1/6883; C07K 16/42; A61K 39/395; A61K 45/06; A61P 11/06; G01N 33/573; A61K 31/498; A61K 31/4985; A61K 31/522; A61K 31/505; A61K 31/519

(52) U.S. Cl. CPC C12Q 1/6883; C07K 16/4291; A61K 39/39566; A61K 45/06; A61P 11/06; G01N 33/573; A61K 2039/505; A61K 31/4985; A61K 31/522; A61K 31/505; A61K 31/519; C12Q 2600/106; A61K 31/498

(57) ABSTRACT

The present invention features, inter alia, methods of treating patients having a mast cell-mediated inflammatory disease, methods of determining whether patients having a mast cell-mediated inflammatory disease are likely to respond to a therapy (e.g., a therapy comprising an agent selected from the group consisting of a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, and a combination thereof), methods of selecting a therapy for a patient having a mast cell-mediated inflammatory disease, methods for assessing a response of a patient having mast cell-mediated inflammatory disease, and methods for monitoring the response of a patient having a mast cell-mediated inflammatory disease.

### CROSS-REFERENCE TO RELATED APPLICATIONS

This application is a continuation of International Application No. PCT/US2019/017320 filed on Feb. 8, 2019, which claims benefit to U.S. Provisional Application No. 62/628,564, filed on Feb. 9, 2018, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Aug. 6, 2020, is named 50474-161002_Sequence_Listing_8.5.20_ST25 and is 47,443 bytes in size.

### FIELD OF THE INVENTION

The present invention relates to therapeutic and diagnostic methods for mast cell-mediated inflammatory diseases, including asthma.

### BACKGROUND

Asthma has canonically been described as an allergic inflammatory disorder of the airways, characterized clinically by episodic, reversible airway obstruction. The therapeutic rationale for targeting mediators of allergic inflammation in asthma has been borne out by the clinical efficacy achieved by anti-Type 2 cytokine therapies, e.g. anti-IL-S. These studies have supported the therapeutic strategy of targeting the Type 2 pathway to provide meaningful clinical benefit, especially in subjects selected on the basis of Type 2 biomarkers. Despite these advances, substantial interest remains to discover and develop new asthma therapies having greater efficacy in Type 2HIGH asthma as well as for asthma patients with low levels of Type 2 biomarkers, for whom currently developed therapies are anticipated to provide less clinical benefit.

Mast cell infiltration of airway smooth muscles is a defining pathophysiologic feature of asthma. IgE/FcεRI-dependent and IgE/FcεRI-independent mechanisms instigate the release of soluble mast cell asthma mediators. Demonstrating the therapeutic importance of targeting mast cell biology, XOLAIR® (omalizumab), an anti-IgE monoclonal antibody therapy, is effective at reducing asthma exacerbations.

There remains a need in the art for improved therapeutic and diagnostic approaches for asthma and other mast cell-mediated inflammatory diseases.

### SUMMARY OF THE INVENTION

The present invention features, inter alia, methods of treating patients having a mast cell-mediated inflammatory disease, methods of determining whether patients having a mast cell-mediated inflammatory disease are likely to respond to a therapy (e.g., a therapy comprising an agent selected from the group consisting of a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, and a combination thereof), methods of selecting a therapy for a patient having a mast cell-mediated inflammatory disease, methods for assessing a response of a patient having mast cell-mediated inflammatory disease, and methods for monitoring the response of a patient having a mast cell-mediated inflammatory disease.

In one aspect, the invention features a method of treating a patient having a mast cell-mediated inflammatory disease who has been identified as having (i) a genotype comprising an active tryptase allele count that is at or above a reference active tryptase allele count; or (ii) an expression level of tryptase in a sample from the patient that is at or above a reference level of tryptase, the method comprising administering to a patient having a mast cell-mediated inflammatory disease a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof.

In another aspect, the invention features a method of determining whether a patient having a mast cell-mediated inflammatory disease is likely to respond to a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof, the method comprising: (a) determining in a sample from a patient having a mast cell-mediated inflammatory disease the patient's active tryptase allele count; and (b) identifying the patient as likely to respond to a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof based on the patient's active tryptase allele count, wherein an active tryptase allele count at or above a reference active tryptase allele count indicates that the patient has an increased likelihood of being responsive to the therapy.

In another aspect, the invention features a method of determining whether a patient having a mast cell-mediated inflammatory disease is likely to respond to a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof, the method comprising: (a) determining the expression level of tryptase in a sample from a patient having a mast cell-mediated inflammatory disease; and (b) identifying the patient as likely to respond to a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof based on the expression level of tryptase in the sample from the patent, wherein an expression level of tryptase in the sample at or above a reference level of tryptase indicates that the patient has an increased likelihood of being responsive to the therapy.

In some embodiments of any of the preceding aspects, the method further comprises administering the therapy to the patient.

In some embodiments of any of the preceding aspects, the patient has been identified as having a level of a Type 2 biomarker in a sample from the patient that is below a reference level of the Type 2 biomarker. In some embodiments, the agent is administered to the patient as a monotherapy.

In some embodiments of any of the preceding aspects, the patient has been identified as having a level of a Type 2 biomarker in a sample from the patient that is at or above a reference level of the Type 2 biomarker. In some embodiments, the method further comprises administering a T_{H}2 pathway inhibitor to the patient.

In another aspect, the invention features a method of treating a patient having a mast cell-mediated inflammatory disease who has been identified as having (i) a genotype comprising an active tryptase allele count that is below a reference active tryptase allele count; or (ii) an expression level of tryptase in a sample from the patient that is below a reference level of tryptase, the method comprising administering to a patient having a mast cell-mediated inflammatory disease a therapy comprising an IgE antagonist or an Fc epsilon receptor (FcεR) antagonist.

In another aspect, the invention features a method of determining whether a patient having a mast cell-mediated inflammatory disease is likely to respond to a therapy comprising an IgE antagonist or an FcεR antagonist, the method comprising: (a) determining in a sample from a patient having a mast cell-mediated inflammatory disease the patient's active tryptase allele count; and (b) identifying the patient as likely to respond to a therapy comprising an IgE antagonist or an FcεR antagonist based on the patient's active tryptase allele count, wherein an active tryptase allele count below a reference active tryptase allele count indicates that the patient has an increased likelihood of being responsive to the therapy.

In another aspect, the invention features a method of determining whether a patient having a mast cell-mediated inflammatory disease is likely to respond to a therapy comprising an IgE antagonist or an FcεR antagonist, the method comprising: (a) determining the expression level of tryptase in a sample from a patient having a mast cell-mediated inflammatory disease; and (b) identifying the patient as likely to respond to a therapy comprising an IgE antagonist or an FcεR antagonist based on the expression level of tryptase in the sample from the patient, wherein an expression level of tryptase in the sample from the patient below a reference level of tryptase indicates that the patient has an increased likelihood of being responsive to the therapy.

In some embodiments of any of the preceding aspects, the method further comprises administering the therapy to the patient.

In some embodiments of any of the preceding aspects, the patient has been identified as having a level of a Type 2 biomarker in a sample from the patient that is at or above a reference level of the Type 2 biomarker. In some embodiments, the method further comprises administering an additional T_{H}2 pathway inhibitor to the patient.

In another aspect, the invention features a method of selecting a therapy for a patient having a mast cell-mediated inflammatory disease, the method comprising: (a) determining in a sample from a patient having a mast cell-mediated inflammatory disease the patient's active tryptase allele count; and (b) selecting for the patient: (i) a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof if the patient's active tryptase allele count is at or above a reference active tryptase allele count, or (ii) a therapy comprising an IgE antagonist or an FcεR antagonist if the patient's active tryptase allele count is below a reference active tryptase allele count.

In another aspect, the invention features a method of selecting a therapy for a patient having a mast cell-mediated inflammatory disease, the method comprising: (a) determining the expression level of tryptase in a sample from a patient having a mast cell-mediated inflammatory disease; and (b) selecting for the patient: (i) a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof if the expression level of tryptase in the sample from the patient is at or above a reference level of tryptase, or (ii) a therapy comprising an IgE antagonist or an FcεR antagonist if the expression level of tryptase in the sample from the patient is below a reference level of tryptase.

In some embodiments of any of the preceding aspects, the method further comprises administering the therapy selected in accordance with (b) to the patient.

In some embodiments of any of the preceding aspects, the patient has been identified as having a level of a Type 2 biomarker in a sample from the patient that is below a reference level of the Type 2 biomarker. In some embodiments, the agent is administered to the patient as a monotherapy.

In some embodiments of any of the preceding aspects, the patient has been identified as having a level of a Type 2 biomarker in a sample from the patient that is at or above a reference level of the Type 2 biomarker, and the method further comprises selecting a combination therapy that comprises a T_{H}2 pathway inhibitor. In some embodiments, the method further comprises administering a T_{H}2 pathway inhibitor (or an additional T_{H}2 pathway inhibitor) to the patient.

In another aspect, the invention features a method for assessing a response of a patient having a mast cell-mediated inflammatory disease to treatment with a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof, the method comprising: (a) determining the expression level of tryptase in a sample from a patient having a mast cell-mediated inflammatory disease at a time point during or after administration of a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof to the patient; and (b) maintaining, adjusting, or stopping the treatment based on a comparison of the expression level of tryptase in the sample with a reference level of tryptase, wherein a change in the expression level of tryptase in the sample from the patient compared to the reference level is indicative of a response to treatment with the therapy. In some embodiments, the change is an increase in the expression level of tryptase and the treatment is maintained. In some embodiments, the change is a decrease in the expression level of tryptase and the treatment is adjusted or stopped.

In another aspect, the invention features a method for monitoring the response of a patient having a mast cell-mediated inflammatory disease treated with a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof, the method comprising: (a) determining the expression level of tryptase in a sample from the patient at a time point during or after administration of the therapy comprising an agent selected from the group consisting of a tryptase antagonist, an IgE antagonist, an IgE⁺ B cell depleting antibody, a mast cell or basophil depleting antibody, a PAR2 antagonist, and a combination thereof to the patient; and (b) comparing the expression level of tryptase in the sample from the patient with a reference level of tryptase, thereby monitoring the response of the patient undergoing treatment with the therapy. In some embodiments, the change is an increase in the level of tryptase and the treatment is maintained. In some embodiments, the change is a decrease in the expression level of tryptase and the treatment is adjusted or stopped.

In another aspect, the invention features an agent selected from the group consisting of a tryptase antagonist, an IgE antagonist, an IgE+ B cell depleting antibody, a mast cell or basophil depleting antibody, a PAR2 antagonist, and a combination thereof for use in a method of treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor. In some embodiments, the tryptase antagonist is an anti-tryptase antibody, e.g., any of the anti-tryptase antibodies disclosed herein. In some embodiments, the IgE antagonist is an anti-IgE antibody. e.g., any of the anti-IgE antibodies disclosed herein.

In another aspect, the invention features an agent selected from an IgE antagonist or an FcεR antagonist for use in a method of treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.

In another aspect, the invention provides for the use of an agent selected from the group consisting of a tryptase antagonist, an IgE antagonist, an IgE+ B cell depleting antibody, a mast cell or basophil depleting antibody, a PAR2 antagonist, and a combination thereof in the manufacture of a medicament for treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor. In some embodiments, the tryptase antagonist is an anti-tryptase antibody, e.g., any of the anti-tryptase antibodies disclosed herein. In some embodiments, the IgE antagonist is an anti-IgE antibody. e.g., any of the anti-IgE antibodies disclosed herein. In some embodiments, the tryptase antagonist is to be administered in combination with an IgE antagonist. In some embodiments, the agent is a tryptase antagonist, and the medicament is formulated for administration with an IgE antagonist.

In another aspect, the invention provides for the use of an IgE antagonist or an FcεR antagonist in the manufacture of a medicament for treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.

In some embodiments of any of the preceding aspects, the active tryptase allele count is determined by sequencing the TPSAB1 and TPSB2 loci of the patient's genome. In some embodiments, the sequencing is Sanger sequencing or massively parallel sequencing. In some embodiments, the TPSAB1 locus is sequenced by a method comprising (i) amplifying a nucleic acid from the subject in the presence of a first forward primer comprising the nucleotide sequence of 5′-CTG GTG TGC AAG GTG AAT GG-3′ (SEQ ID NO: 31) and a first reverse primer comprising the nucleotide sequence of 5′-AGG TOO AGO ACT CAG GAG GA-3′ (SEQ ID NO: 32) to form a TPSAB1 amplicon, and (ii) sequencing the TPSAB1 amplicon. In some embodiments, sequencing the TPSAB1 amplicon comprises using the first forward primer and the first reverse primer. In some embodiments, the TPSB2 locus is sequenced by a method comprising (i) amplifying a nucleic acid from the subject in the presence of a second forward primer comprising the nucleotide sequence of 5′-GCA GGT GAG OCT GAG AGT CO-3′ (SEQ ID NO: 33) and a second reverse primer comprising the nucleotide sequence of 5″-GGG ACC TTC ACC TGC TTC AG-3′ (SEQ ID NO: 34) to form a TPSB2 amplicon, and (ii) sequencing the TPSB2 amplicon. In some embodiments, sequencing the TPSB2 amplicon comprises using the second forward primer and a sequencing reverse primer comprising the nucleotide sequence of 5′-CAG CCA GTG ACC CAG CAC-3′ (SEQ ID NO: 35).

In some embodiments of any of the preceding aspects, the active tryptase allele count is determined by the formula: 4—the sum of the number of tryptase α and tryptase β III frame-shift (βIII^{FS}) alleles in the patient's genotype. In some embodiments, tryptase alpha is detected by detecting the c733 G>A SNP at TPSAB1 comprising the nucleotide sequence CTGCAGGCGGGCGTGGTCAGCTGGG[G/A]CGAGGGCTGTGCCCAGCCCAACCGG (SEQ ID NO: 36), wherein the presence of an A at the c733 G>A SNP indicates tryptase alpha. In some embodiments, tryptase beta III^{FS} is detected by detecting a c980_981insC mutation at TPSB2 comprising the nucleotide sequence CACACGGTCACCCTGCCCCCTGCCTCAGAGACCTTCCCCCCC (SEQ ID NO: 37).

In some embodiments of any of the preceding aspects, the reference active tryptase allele count is determined in a group of patients having the mast cell-mediated inflammatory disease. In some embodiments, the reference active tryptase allele count is 3.

In some embodiments of any of the preceding aspects, the patient has an active tryptase allele count of 3 or 4.

In some embodiments of any of the preceding aspects, the patient has an active tryptase allele count of 0, 1, or 2.

In some embodiments of any of the preceding aspects, the tryptase is tryptase beta I, tryptase beta II, tryptase beta III, tryptase alpha I, or a combination thereof.

In some embodiments of any of the preceding aspects, the expression level of tryptase is a protein expression level. In some embodiments, the protein expression level of tryptase is an expression level of active tryptase. In some embodiments, the protein expression level of tryptase is an expression level of total tryptase. In some embodiments, the protein expression level is measured using an immunoassay, enzyme-linked immunosorbent assay (ELISA), Western blot, or mass spectrometry. In some embodiments, the expression level of the tryptase is an mRNA expression level. In some embodiments, the mRNA expression level is measured using a polymerase chain reaction (PCR) method or a microarray chip. In some embodiments, the PCR method is qPCR.

In some embodiments of any of the preceding aspects, the reference level of tryptase is a level determined in a group of individuals having the mast cell-mediated inflammatory disease. In some embodiments, the reference level of tryptase is a median level.

In some embodiments of any of the preceding aspects, the sample from the patient is selected from the group consisting of a blood sample, a tissue sample, a sputum sample, a bronchiolar lavage sample, a mucosal lining fluid (MLF) sample, a bronchosorption sample, and a nasosorption sample. In some embodiments, the blood sample is a whole blood sample, a serum sample, a plasma sample, or a combination thereof. In some embodiments, the blood sample is a serum sample or a plasma sample.

In some embodiments of any of the preceding aspects, the agent is a tryptase antagonist. In some embodiments, the tryptase antagonist is a tryptase alpha antagonist or a tryptase beta antagonist. In some embodiments, the tryptase antagonist is a tryptase beta antagonist. In some embodiments, the tryptase beta antagonist is an anti-tryptase beta antibody or an antigen-binding fragment thereof. In some embodiments, the antibody comprises the following six hypervariable regions (HVRs): (a) an HVR-H1 comprising the amino acid sequence of DYGMV (SEQ ID NO: 1); (b) an HVR-H2 comprising the amino acid sequence of FISSGSSTVYYADTMKG (SEQ ID NO: 2); (c) an HVR-H3 comprising the amino acid sequence of RNYDDWYFDV (SEQ ID NO: 3); (d) an HVR-L1 comprising the amino acid sequence of SASSSVTYMY (SEQ ID NO: 4); (e) an HVR-L2 comprising the amino acid sequence of RTSDLAS (SEQ ID NO: 5); and (f) an HVR-L3 comprising the amino acid sequence of QHYHSYPLT (SEQ ID NO: 6). In some embodiments, the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b). In some embodiments, the VH domain comprises the amino acid sequence of SEQ ID NO: 7. In some embodiments, the VL domain comprises the amino acid sequence of SEQ ID NO: 8. In some embodiments, the VH domain comprises the amino acid sequence of SEQ ID NO: 7 and the VL domain comprises the amino acid sequence of SEQ ID NO: 8. In some embodiments, the antibody comprises (a) a heavy chain comprising the amino acid sequence of SEQ ID NO: 9 and (b) a light chain comprising the amino acid sequence of SEQ ID NO: 10. In some embodiments, the antibody comprises (a) a heavy chain comprising the amino acid sequence of SEQ ID NO: 11 and (b) a light chain comprising the amino acid sequence of SEQ ID NO: 10. In some embodiments, the antibody comprises the following six HVRs: (a) an HVR-H1 comprising the amino acid sequence of GYAIT (SEQ ID NO: 12); (b) an HVR-H2 comprising the amino acid sequence of GISSAATTFYSSWAKS (SEQ ID NO: 13); (c) an HVR-H3 comprising the amino acid sequence of DPRGYGAALDRLDL (SEQ ID NO: 14); (d) an HVR-L1 comprising the amino acid sequence of QSIKSVYNNRLG (SEQ ID NO: 15); (e) an HVR-L2 comprising the amino acid sequence of ETSILTS (SEQ ID NO: 16); and (f) an HVR-L3 comprising the amino acid sequence of AGGFDRSGDTT (SEQ ID NO: 17). In some embodiments, the antibody comprises (a) a heavy chain variable (VH) domain comprising an amino acid sequence having at least 90%, at least 95%, or at least 99% sequence identity to the amino acid sequence of SEQ ID NO: 18; (b) a light chain variable (VL) domain comprising an amino acid sequence having at least 90%, at least 95%, or at least 99% identity to the amino acid sequence of SEQ ID NO: 19; or (c) a VH domain as in (a) and a VL domain as in (b). In some embodiments, the VH domain comprises the amino acid sequence of SEQ ID NO: 18. In some embodiments, the VL domain comprises the amino acid sequence of SEQ ID NO: 19. In some embodiments, the VH domain comprises the amino acid sequence of SEQ ID NO: 18 and the VL domain comprises the amino acid sequence of SEQ ID NO: 19. In some embodiments, the antibody comprises (a) a heavy chain comprising the amino acid sequence of SEQ ID NO: 20 and (b) a light chain comprising the amino acid sequence of SEQ ID NO: 21. In some embodiments, the antibody comprises (a) a heavy chain comprising the amino acid sequence of SEQ ID NO: 22 and (b) a light chain comprising the amino acid sequence of SEQ ID NO: 21. In some embodiments, the therapy further comprises an IgE antagonist.

In some embodiments of any of the preceding aspects, the agent is an FcεR antagonist. In some embodiments, the FcεR antagonist is a Bruton's tyrosine kinase (BTK) inhibitor. In some embodiments, the BTK inhibitor is GDC-0853, acalabrutinib, GS-4059, spebrutinib, BGB-3111, or HM71224. In some embodiments, the agent is an IgE⁺ B cell depleting antibody. In some embodiments, the IgE⁺ B cell depleting antibody is an anti-M1′ domain antibody.

In some embodiments of any of the preceding aspects, the agent is a mast cell or basophil depleting antibody.

In some embodiments of any of the preceding aspects, the agent is a PAR2 antagonist.

In some embodiments of any of the aspects disclosed herein, the therapy or the combination comprises a tryptase antagonist (e.g., an anti-tryptase antibody, including any of the anti-tryptase antibodies described herein) and an IgE antagonist (e.g., an anti-IgE antibody, including any of the anti-IgE antibodies described herein, e.g., omalizumab (e.g., XOLAIR®)).

In some embodiments of any of the aspects disclosed herein, the agent is an IgE antagonist. In some embodiments, the IgE antagonist is an anti-IgE antibody. In some embodiments, the anti-IgE antibody is an IgE blocking antibody and/or an IgE depleting antibody. In some embodiments, the anti-IgE antibody comprises the following six HVRs: (a) an HVR-H1 comprising the amino acid sequence of GYSWN (SEQ ID NO: 40); (b) an HVR-H2 comprising the amino acid sequence of SITYDGSTNYNPSVKG (SEQ ID NO: 41); (c) an HVR-H3 comprising the amino acid sequence of GSHYFGHWHFAV (SEQ ID NO: 42); (d) an HVR-L1 comprising the amino acid sequence of RASQSVDYDGDSYMN (SEQ ID NO: 43); (e) an HVR-L2 comprising the amino acid sequence of AASYLES (SEQ ID NO: 44); and (f) an HVR-L3 comprising the amino acid sequence of QQSHEDPYT (SEQ ID NO: 45). In some embodiments, the anti-IgE antibody comprises (a) a heavy chain variable (VH) domain comprising an amino acid sequence having at least 90%, at least 95%, or at least 99% sequence identity to the amino acid sequence of SEQ ID NO: 38; (b) a light chain variable (VL) domain comprising an amino acid sequence having at least 90%, at least 95%, or at least 99% identity to the amino acid sequence of SEQ ID NO: 39; or (c) a VH domain as in (a) and a VL domain as in (b). In some embodiments, the VH domain comprises the amino acid sequence of SEQ ID NO: 38. In some embodiments, the VL domain comprises the amino acid sequence of SEQ ID NO: 39. In some embodiments, the VH domain comprises the amino acid sequence of SEQ ID NO: 38 and the VL domain comprises the amino acid sequence of SEQ ID NO: 39. In some embodiments, the anti-IgE antibody is omalizumab (XOLAIR®) or XmAb7195. In some embodiments, the anti-IgE antibody is omalizumab (XOLAIR®).

In some embodiments of any of the preceding aspects, the Type 2 biomarker is a T_{H}2 cell-related cytokine, periostin, eosinophil count, an eosinophil signature, FeNO, or IgE. In some embodiments, the T_{H}2 cell-related cytokine is IL-13, IL-4, IL-9, or IL-5. In some embodiments, the T_{H}2 pathway inhibitor inhibits any of the targets selected from interleukin-2-inducible T cell kinase (ITK), Bruton's tyrosine kinase (BTK), Janus kinase 1 (JAK1) (e.g., ruxolitinib, tofacitinib, oclacitinib, baricitinib, filgotinib, gandotinib, lestaurtinib, momelotinib, pacrinitib, upadacitinib, peficitinib, and fedratinib), GATA binding protein 3 (GATA3), IL-9 (e.g., MEDI-528), IL-5 (e.g., mepolizumab, CAS No. 196078-29-2; resilizumab), IL-13 (e.g., IMA-026, IMA-638 (also referred to as anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody)), IL-4 (e.g., AER-001, IL-4/IL-13 trap), OX40L, TSLP, IL-25, IL-33, and IgE (e.g., XOLAIR®, QGE-031; and MEDI-4212); and receptors such as: IL-9 receptor, IL-5 receptor (e.g., MEDI-563 (benralizumab, CAS No. 1044511-01-4)), IL-4 receptor alpha (e.g., AMG-317, AIR-645), IL-13 receptoralphal (e.g., R-1671) and IL-13 receptoralpha2, OX40, TSLP-R, IL-7Ralpha (a co-receptor for TSLP), IL-17RB (receptor for IL-25), ST2 (receptor for IL-33), CCR3, CCR4, CRTH2 (e.g., AMG-853, AP768, AP-761, MLN6095, ACT129968), FcεRI, FcεRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL-5, IL-3, and GM-CSF (e.g., TPI ASM8).

In some embodiments of any of the preceding aspects, the method further comprises administering an additional therapeutic agent to the patient. In some embodiments, the additional therapeutic agent is selected from the group consisting of a corticosteroid, an IL-33 axis binding antagonist, a TRPA1 antagonist, a bronchodilator or asthma symptom control medication, an immunomodulator, a tyrosine kinase inhibitor, and a phosphodiesterase inhibitor. In some embodiments, the additional therapeutic agent is a corticosteroid. In some embodiments, the corticosteroid is an inhaled corticosteroid.

In some embodiments of any of the preceding aspects, the mast cell-mediated inflammatory disease is selected from the group consisting of asthma, atopic dermatitis, chronic spontaneous urticaria (CSU), systemic anaphylaxis, mastocytosis, chronic obstructive pulmonary disease (COPD), idiopathic pulmonary fibrosis (IPF), and eosinophilic esophagitis. In some embodiments, the mast cell-mediated inflammatory disease is asthma. In some embodiments, the asthma is moderate to severe asthma. In some embodiments, the asthma is uncontrolled on a corticosteroid. In some embodiments, the asthma is T_{H}2 high asthma or T_{H}2 low asthma.

In another aspect, the invention features a kit for identifying a patient having a mast cell-mediated inflammatory disease who is likely to respond to a therapy comprising an agent selected from the group consisting of a tryptase antagonist, an IgE antagonist, an IgE+ B cell depleting antibody, a mast cell or basophil depleting antibody, a protease activated receptor 2 (PAR2) antagonist, and a combination thereof, the kit comprising: (a) reagents for determining the patient's active tryptase allele count or for determining the expression level of tryptase in a sample from the patient; and, optionally, (b) instructions for using the reagents to identify a patient having a mast cell-mediated inflammatory disease who is likely to respond to a therapy comprising an agent selected from the group consisting of a tryptase antagonist, an IgE antagonist, an IgE+ B cell depleting antibody, a mast cell or basophil depleting antibody, a PAR2 antagonist, and a combination thereof. In some embodiments, the agent is a tryptase antagonist, and the therapy further comprises an IgE antagonist. In some embodiments, the therapy comprises a tryptase antagonist and an IgE antagonist.

In another aspect, the invention features a kit for identifying a patient having a mast cell-mediated inflammatory disease who is likely to respond to a therapy comprising an IgE antagonist or an FcεR antagonist, the kit comprising: (a) reagents for determining the patient's active tryptase allele count or for determining the expression level of tryptase in a sample from the patient; and, optionally, (b) instructions for using the reagents to identify a patient having a mast cell-mediated inflammatory disease who is likely to respond to a therapy comprising an IgE antagonist or an FcεR antagonist.

In some embodiments of any of the preceding aspects, the kit further comprises reagents for determining the level of a Type 2 biomarker in a sample from the patient.

In another aspect, the invention features an agent selected from the group consisting of a tryptase antagonist, an IgE antagonist, an IgE+ B cell depleting antibody, a mast cell or basophil depleting antibody, a PAR2 antagonist, and a combination thereof for use in a method of treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor.

In another aspect, the invention features an agent selected from an IgE antagonist or an FcεR antagonist for use in a method of treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.

In another aspect, the invention provides for the use of an agent selected from the group consisting of a tryptase antagonist, an IgE antagonist, an IgE+ B cell depleting antibody, a mast cell or basophil depleting antibody, a PAR2 antagonist, and a combination thereof in the manufacture of a medicament for treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor.

In another aspect, the invention provides for the use of an IgE antagonist or an FcεR antagonist in the manufacture of a medicament for treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.

### BRIEF DESCRIPTION OF THE DRAWINGS

FIG. 1 is a graph showing active tryptase allele count for moderate to severe asthma patients. Active tryptase allele count is plotted by barplot for BOBCAT, EXTRA, and MILLY moderate to severe asthma subjects.

FIGS. 2A and 2B are a series of graphs showing that total peripheral tryptase protein level is associated with tryptase copy number in moderate to severe asthma. Protein Quantitative Trait Linkage (pQTL) analyses were conducted for plasma total tryptase from BOBCAT (FIG. 2A) and serum total tryptase from MILLY studies (FIG. 2B). Linear regression line (95% CI) are indicated in gray shading. The P-value of r² from linear regression is annotated on the plots. r² is the coefficient of determination of the linear regression, which takes on a value from 0 to 1; increasing values indicate the proportion of variance described by the independent variable.

FIG. 3 is a series of graphs showing asthmatic FEV₁ treatment benefit from anti-IgE therapy (omalizumab (XOLAIR®)) based on active tryptase copy number. FEV₁ percent change from baseline was assessed in subjects from the EXTRA study on the basis of active tryptase allele count (left panel, 1 or 2; right panel, 3 or 4).

FIGS. 4A-4C are a series of graphs showing that biomarkers of Type 2 asthma do not correlate with active tryptase allele count in moderate to severe asthma. The levels of the Type 2 biomarkers serum periostin (FIG. 4A), fractional exhaled nitric oxide (FeNO) (FIG. 4B), and blood eosinophil count (FIG. 4C) were assessed with respect to active tryptase count in BOBCAT, EXTRA, and MILLY moderate to severe asthma cohorts.

### DETAILED DESCRIPTION OF EMBODIMENTS OF THE INVENTION

### I. Definitions

The term “about” as used herein refers to the usual error range for the respective value readily known to the skilled person in this technical field. Reference to “about” a value or parameter herein includes (and describes) embodiments that are directed to that value or parameter per se.

The terms “biomarker” and “marker” are used interchangeably herein to refer to a DNA, RNA, protein, carbohydrate, or glycolipid-based molecular marker, the expression or presence of which in a subject's or patient's sample can be detected by standard methods (or methods disclosed herein) and is useful, for example, for identifying, for example, the likelihood of responsiveness or sensitivity of a mammalian subject to a treatment, or for monitoring the response of a subject to a treatment. Expression of such a biomarker may be determined to be higher or lower in a sample obtained from a patient that has an increased or decreased likelihood of being responsive to a therapy than a reference level (including, e.g., the median expression level of the biomarker in samples from a group/population of patients (e.g., asthma patients); the level of the biomarker in samples from a group/population of control individuals (e.g., healthy individuals); or the level in a sample previously obtained from the individual at a prior time). In particular embodiments, a biomarker as described herein is an active tryptase allele count or an expression level of tryptase.

As used herein, “tryptase” refers to any native tryptase from any vertebrate source, including mammals such as primates (e.g., humans) and rodents (e.g., mice and rats), unless otherwise indicated. Tryptase is also known in the art as mast cell tryptase, mast cell protease II, skin tryptase, lung tryptase, pituitary tryptase, mast cell neutral proteinase, and mast cell serine proteinase II. The term “tryptase” encompasses tryptase alpha (encoded in humans by TPSAB1), tryptase beta (encoded in humans by TPSAB1 and TPSB2; see below), tryptase delta (encoded in humans by TPSD1), tryptase gamma (encoded in humans by TPSG1), and tryptase epsilon (encoded in humans by PRSS22). Tryptase alpha (α), beta (β), and gamma (γ) proteins are soluble, whereas tryptase epsilon (ε) proteins are membrane anchored. Tryptase beta and gamma are active serine proteases, although they have different specificities. Tryptase alpha and delta (δ) proteins are largely inactive proteases as they have residues in critical position that differ from typical active serine proteases. An exemplary tryptase alpha full length protein sequence can be found under NCBI GenBank Accession No. ACZ98910.1. Exemplary tryptase gamma full length protein sequences can be found under Uniprot Accession No. Q9NRR2 or GenBank Accession Nos. Q9NRR2.3, AAF03695.1, NP_036599.3 or AAF76457.1. Exemplary tryptase delta full length protein sequences can be found under Uniprot Accession No. Q9BZJ3 or GenBank Accession No. NP_036349.1. Several tryptase genes are clustered on human chromosome 16p13.3. The term encompasses “full-length,” unprocessed tryptase as well as any form of tryptase that results from processing in the cell. Tryptase beta is the main tryptase expressed in mast cells, while tryptase alpha is the main tryptase expressed in basophils. Tryptase alpha and tryptase beta typically include a leader sequence of approximately 30 amino acids and a catalytic sequence of approximately 245 amino acids (see, e.g., Schwartz, Immunol. Allergy Clin. N. Am. 26:451-463, 2006).

As used herein, “tryptase beta” refers to any native tryptase beta from any vertebrate source, including mammals such as primates (e.g., humans) and rodents (e.g., mice and rats), unless otherwise indicated. Tryptase beta is a serine protease that is a major constituent of mast cell secretory granules. As used herein, the term encompasses tryptase beta 1 (encoded by the TPSAB1 gene, which also encodes tryptase alpha 1), tryptase beta 2 (encoded by the TPSB2 gene), and tryptase beta 3 (also encoded by the TPSB2 gene). An exemplary human tryptase beta 1 sequence is shown in SEQ ID NO: 23 (see also GenBank Accession No. NP_003285.2). An exemplary human tryptase beta 2 sequence is shown in SEQ ID NO: 24 (see also GenBank Accession No. AAD13876.1). An exemplary human tryptase beta 3 sequence is shown in SEQ ID NO: 25 (see also GenBank Accession No. NP_077078.5). The term tryptase beta encompasses “full-length,” unprocessed tryptase beta as well as tryptase beta that results from post-translational modifications, including proteolytic processing. Full-length, pro-tryptase beta is thought to be processed in two proteolytic steps. First, autocatalytic intermolecular cleavage at R⁻³ occurs, particularly at acidic pH and in the presence of a polyanion (e.g., heparin or dextran sulfate). Next, the remaining pro′ dipeptide is removed (likely by dipeptidyl peptidase I). For full-length human tryptase beta 1, with reference to SEQ ID NO: 23 below, the underlined amino acid residues correspond to the native leader sequence, and the bolded and gray-shaded amino acid residues correspond to the pro-domain, which are cleaved to form the mature protein (see, e.g., Sakai et al. J. Clin. Invest. 97:988-995, 1996)

(SEQ ID NO: 23)

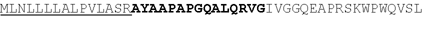

RVHGPYMHFCGGSLIHPQWVLTAAHCVGPDVKDLAALRVQLREQHLYY
QDQLLPVSRIIVHPQFYTAQIGADIALLELEEPVNVSSHVHTVTLPPA
SETFPPGMPCWVTGWGDVDNDERLPPPFPLKQVKVPIMENHICDAKYH
LGAYTGDDVRIVRDDMLCAGNTRRDSCQGDSGGPLVCKVNGTWLQAGV
VSWGEGCAQPNRPGIYTRVTYYLDWIHHYVPKKP.

Mature, enzymatically active tryptase beta is typically a homotetramer or heterotetramer, although active monomer has been reported (see, e.g., Fukuoka et al. J. Immunol. 176:3165, 2006). The subunits of the tryptase beta tetramer are held together by hydrophobic and polar interactions between subunits and stabilized by polyanions (particularly heparin and dextran sulfate). The term tryptase can refer to tryptase tetramer or tryptase monomer. Exemplary sequences for mature human tryptase beta 1, beta 2, and beta 3 are shown in SEQ ID NO: 26, SEQ ID NO: 27, and SEQ ID NO: 28, respectively. The active site of each subunit faces into a central pore of the tetramer, which measures approximately 50×30 angstroms (see, e.g., Pereira et al. Nature 392:306-311, 1998). The size of the central pore typically restricts access of the active sites by inhibitors. Exemplary substrates of tryptase beta include, but are not limited to, PAR2, C3, fibrinogen, fibronectin, and kininogen.

The terms “oligonucleotide” and “polynucleotide” are used interchangeably and refer to a molecule comprised of two or more deoxyribonucleotides or ribonucleotides, preferably more than three. Its exact size will depend on many factors, which in turn depend on the ultimate function or use of the oligonucleotide. An oligonucleotide can be derived synthetically or by cloning. Chimeras of deoxyribonucleotides and ribonucleotides may also be in the scope of the present invention.

The term “genotype” refers to a description of the alleles of a gene contained in an individual or a sample. In the context of this invention, no distinction is made between the genotype of an individual and the genotype of a sample originating from the individual. Although typically a genotype is determined from samples of diploid cells, a genotype can be determined from a sample of haploid cells, such as a sperm cell.

A nucleotide position in a genome at which more than one sequence is possible in a population is referred to herein as a “polymorphism” or “polymorphic site.” A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region.

The term “single nucleotide polymorphism” or “SNP” refers to a single base substitution within a DNA sequence that leads to genetic variability. Single nucleotide polymorphisms may occur at any region of a gene. In some instances the polymorphism can result in a change in protein sequence. The change in protein sequence may affect protein function or not.

When there are two, three, or four alternative nucleotide sequences at a polymorphic site, each nucleotide sequence is referred to as a “polymorphic variant” or “nucleic acid variant.” Each possible variant in the DNA sequence is referred to as an “allele.” Typically, the first identified allelic form is arbitrarily designated as the reference form and other allelic forms are designated as alternative or variant alleles.

The term “active tryptase allele count” refers to the number of active tryptase alleles in a subject's genotype. In some embodiments, an active tryptase allele count can be inferred by accounting for inactivating mutations of TPSAB1 and TPSB2. Because each diploid subject will have two copies each of TPSAB1 and TPSB2, an active tryptase allele count can be determined according to the formula 4—the sum of the number of tryptase alpha and tryptase beta III frame-shift (beta III^{FS}) alleles in the subject's genotype. In some embodiments, a subject's active tryptase allele count is an integer in the range of from 0 to 4 (e.g., 0, 1, 2, 3, or 4).

The term “reference active tryptase allele count” refers to an active tryptase allele count against which another active tryptase allele count is compared, e.g., to make a diagnostic, predictive, prognostic, and/or therapeutic determination. A reference active tryptase allele count can be determined in a reference sample, a reference population, and/or a pre-assigned value (e.g., a cut-off value which was previously determined to significantly (e.g., statistically significantly) separate a first subset of individuals from a second subset of individuals (e.g., in terms of response to a therapy (e.g., a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof)). In some embodiments, the reference active tryptase allele count is a pre-determined value. The reference active tryptase allele count in one embodiment has been predetermined in the disease entity to which the patient belongs (e.g., a mast cell-mediated inflammatory disease such as asthma). In certain embodiments, the active tryptase allele count is determined from the overall distribution of the values in a disease entity investigated or in a given population. In some embodiments, a reference active tryptase allele count is an integer in the range of from 0 to 4 (e.g., 0, 1, 2, 3, or 4). In particular embodiments, a reference active tryptase allele count is 3.

The terms “level,” “level of expression,” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (e.g., transfer and ribosomal RNAs).

In certain embodiments, the term “reference level” herein refers to a predetermined value. As the skilled artisan will appreciate, the reference level is predetermined and set to meet the requirements in terms of, for example, specificity and/or sensitivity. These requirements can vary, e.g., from regulatory body to regulatory body. It may be, for example, that assay sensitivity or specificity, respectively, has to be set to certain limits, e.g., 80%, 90%, or 95%. These requirements may also be defined in terms of positive or negative predictive values. Nonetheless, based on the teaching given in the present invention it will always be possible to arrive at the reference level meeting those requirements. In one embodiment, the reference level is determined in healthy individuals. The reference value in one embodiment has been predetermined in the disease entity to which the patient belongs (e.g., a mast cell-mediated inflammatory disease such as asthma). In certain embodiments, the reference level can be set to any percentage between, e.g., 25% and 75% of the overall distribution of the values in a disease entity investigated. In other embodiments, the reference level can be set to, for example, the median, tertiles, quartiles, or quintiles as determined from the overall distribution of the values in a disease entity investigated or in a given population. In one embodiment, the reference level is set to the median value as determined from the overall distribution of the values in a disease entity investigated. In one embodiment, the reference level may depend on the gender of the patient, e.g., males and females may have different reference levels.

In certain embodiments, the term “at a reference level” refers to a level of a marker (e.g., tryptase) that is the same as the level, detected by the methods described herein, from a reference sample.

In certain embodiments, the term “increase” or “above” refers to a level at the reference level or to an overall increase of 5%, 10%, 20%, 25%, 30%, 40%, 50%, 60%, 70%, 80%, 85%, 90%, 95%, 100%, or greater, in the level of a marker (e.g., tryptase) detected by the methods described herein, as compared to the level from a reference sample.

In certain embodiments, the term “decrease” or “below” herein refers to a level below the reference level or to an overall reduction of 5%, 10%, 20%, 25%, 30%, 40%, 50%, 60%, 70%, 80%, 85%, 90%, 95%, 96%, 97%, 98%, 99% or greater, in the level of a marker (e.g., tryptase) detected by the methods described herein, as compared to the level from a reference sample.

A “disorder” or “disease” is any condition that would benefit from treatment or diagnosis with a method of the invention. This includes chronic and acute disorders or diseases including those pathological conditions which predispose the mammal to the disorder in question. Examples of disorders to be treated herein include mast cell-mediated inflammatory diseases such as asthma.

A “mast cell-mediated inflammatory disease” refers to a diseases or disorders that are mediated at least in part by mast cells, such as asthma (e.g., allergic asthma), urticaria (e.g., chronic spontaneous urticaria (CSU) or chronic idiopathic urticaria (CIU)), eczema, itch, allergy, atopic allergy, anaphylaxis, anaphylactic shock, allergic bronchopulmonary aspergillosis, allergic rhinitis, allergic conjunctivitis, as well as autoimmune disorders including rheumatoid arthritis, juvenile rheumatoid arthritis, psoriatic arthritis, pancreatitis, psoriasis, plaque psoriasis, guttate psoriasis, inverse psoriasis, pustular psoriasis, erythrodermic psoriasis, paraneoplastic autoimmune diseases, autoimmune hepatitis, bullous pemphigoid, myasthenia gravis, inflammatory bowel disease, Crohn's disease, ulcerative colitis, celiac disease, thyroiditis (e.g., Graves' disease), Sjogren's syndrome, Guillain-Barre disease, Raynaud's phenomenon, Addison's disease, liver diseases (e.g., primary biliary cirrhosis, primary sclerosing cholangitis, non-alcoholic fatty liver disease, and non-alcoholic steatohepatitis), and diabetes (e.g., type I diabetes).

In some embodiments, the asthma is persistent chronic severe asthma with acute events of worsening symptoms (exacerbations or flares) that can be life threatening. In some embodiments, the asthma is atopic (also known as allergic) asthma, non-allergic asthma (e.g., often triggered by infection with a respiratory virus (e.g., influenza, parainfluenza, rhinovirus, human metapneumovirus, and respiratory syncytial virus) or inhaled irritant (e.g., air pollutants, smog, diesel particles, volatile chemicals and gases indoors or outdoors, or even by cold dry air).

In some embodiments, the asthma is intermittent or exercise-induced, asthma due to acute or chronic primary or second-hand exposure to “smoke” (typically cigarettes, cigars, or pipes), inhaling or “vaping” (tobacco, marijuana, or other such substances), or asthma triggered by recent ingestion of aspirin or related non-steroidal anti-inflammatory drugs (NSAIDs). In some embodiments, the asthma is mild, or corticosteroid naïve asthma, newly diagnosed and untreated asthma, or not previously requiring chronic use of inhaled topical or systemic steroids to control the symptoms (cough, wheeze, shortness of breath/breathlessness, or chest pain). In some embodiments, the asthma is chronic, corticosteroid resistant asthma, corticosteroid refractory asthma, asthma uncontrolled on corticosteroids or other chronic asthma controller medications.

In some embodiments, the asthma is moderate to severe asthma. In certain embodiments, the asthma is T_{H}2-high asthma. In some embodiments, the asthma is severe asthma. In some embodiments, the asthma is atopic asthma, allergic asthma, non-allergic asthma (e.g., due to infection and/or respiratory syncytial virus (RSV)), exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids. In some embodiments, the asthma is eosinophilic asthma. In some embodiments, the asthma is allergic asthma. In some embodiments, the individual has been determined to be Eosinophilic Inflammation Positive (EIP). See WO 2015/061441. In some embodiments, the asthma is periostin-high asthma (e.g., having periostin level at least about any of 20 ng/ml, 25 ng/ml, or 50 ng/ml serum). In some embodiments, the asthma is eosinophil-high asthma (e.g., at least about any of 150, 200, 250, 300, 350, 400 eosinophil counts/ml blood). In some embodiments, the individual has been determined to be Eosinophilic Inflammation Negative (EIN). See WO 2015/061441. In some embodiments, the asthma is periostin-low asthma (e.g., having periostin level less than about 20 ng/ml serum). In some embodiments, the asthma is eosinophil-low asthma (e.g., less than about 150 eosinophil counts/μl blood or less than about 100 eosinophil counts/μl blood).

The term “T_{H}2-high asthma,” as used herein, refers to asthma that exhibits high levels of one or more T_{H}2 cell-related cytokines, for example, IL-13, IL-4, IL-9, or IL-5, or that exhibits T_{H}2 cytokine-associated inflammation. In certain embodiments, the term T_{H}2-high asthma may be used interchangeably with eosinophil-high asthma, T helper lymphocyte type 2-high, type 2-high, or T_{H}2-driven asthma. In some embodiments, the asthma patient has been determined to be Eosinophilic Inflammation Positive (EIP). See, e.g., International Patent Application Publication No. WO 2015/061441, which is incorporated by reference herein in its entirety. In certain embodiments, the individual has been determined to have elevated levels of at least one of the eosinophilic signature genes as compared to a control or reference level. See WO 2015/061441. In certain embodiments, the T_{H}2-high asthma is periostin-high asthma. In some embodiments, the individual has high serum periostin. In certain embodiments, the individual is eighteen years or older. In certain embodiments, the individual has been determined to have an elevated level of serum periostin as compared to a control or reference level. In certain embodiments, the control or reference level is the median level of periostin in a population. In certain embodiments, the individual has been determined to have 20 ng/ml or higher serum periostin. In certain embodiments, the individual has been determined to have 25 ng/ml or higher serum periostin. In certain embodiments, the individual has been determined to have 50 ng/ml or higher serum periostin. In certain embodiments, the control or reference level of serum periostin is 20 ng/ml, 25 ng/ml, or 50 ng/ml. In certain embodiments, the asthma is eosinophil-high asthma. In certain embodiments, the individual has been determined to have an elevated eosinophil count as compared to a control or reference level. In certain embodiments, the control or reference level is the median level of a population. In certain embodiments, the individual has been determined to have 150 or higher eosinophil count/μl blood. In certain embodiments, the individual has been determined to have 200 or higher eosinophil count/μl blood. In certain embodiments, the individual has been determined to have 250 or higher eosinophil count/μl blood. In certain embodiments, the individual has been determined to have 300 or higher eosinophil count/μl blood. In certain embodiments, the individual has been determined to have 350 or higher eosinophil count/μl blood. In certain embodiments, the individual has been determined to have 400 or higher eosinophil count/μl blood. In certain embodiments, the individual has been determined to have 450 or higher eosinophil count/μl blood. In certain embodiments, the individual has been determined to have 500 or higher eosinophil count/μl blood. In certain preferred embodiments, the individual has been determined to have 300 or higher eosinophil count/μl blood. In certain embodiments, the eosinophils are peripheral blood eosinophils. In certain embodiments, the eosinophils are sputum eosinophils. In certain embodiments, the individual exhibits elevated level of FeNO (fractional exhaled nitric acid) and/or elevated level of IgE. For example, in some instances, the individual exhibits a FeNO level above about 250 parts per billion (ppb), above about 275 ppb, above about 300 ppb, above about 325 ppb, above about 325 ppb, or above about 350 ppb. In some instances, the individual has an IgE level that is above 50 IU/ml. For a review of T_{H}2-high asthma, see, e.g., Fajt et al. J. Allergy Clin. Immunol. 135(2):299-310, 2015.

The term “T_{H}2-low asthma” or “non-T_{H}2-high asthma” as used herein, refers to asthma that exhibits low levels of one or more T_{H}2 cell-related cytokines, for example, IL-13, IL-4, IL-9, or IL-5, or exhibits non-T_{H}2 cytokine-associated inflammation. In certain embodiments, the term T_{H}2-low asthma may be used interchangeably with eosinophil-low asthma. In some embodiments, the asthma patient has been determined to be Eosinophilic Inflammation Negative (EIN). See, e.g., WO 2015/061441. In certain embodiments, the T_{H}2-low asthma is periostin-low asthma. In certain embodiments, the individual is eighteen years or older. In certain embodiments, the individual has been determined to have a reduced level of serum periostin as compared to a control or reference level. In certain embodiments, the control or reference level is the median level of periostin in a population. In certain embodiments, the individual has been determined to have less than 20 ng/ml serum periostin. In certain embodiments, the asthma is eosinophil-low asthma. In certain embodiments, the individual has been determined to have a reduced eosinophil count as compared to a control or reference level. In certain embodiments, the control or reference level is the median level of a population. In certain embodiments, the individual has been determined to have less than 150 eosinophil count/μl blood. In certain embodiments, the individual has been determined to have less than 100 eosinophil count/μl blood. In certain embodiments, the individual has been determined to have less than 300 eosinophil count/μl blood.

As used herein, a “Type 2 biomarker” refers to a biomarker that is associated with T_{H}2 inflammation. Non-limiting examples of Type 2 biomarkers include a T_{H}2 cell-related cytokine (e.g., IL-13, IL-4, IL-9, or IL-5), periostin, eosinophil count, an eosinophil signature, FeNO, or IgE.

The term “administering” means the administration of a composition to a patient (e.g., a patient having a mast cell-mediated inflammatory disease such as asthma). The compositions utilized in the methods described herein can be administered, for example, parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. Parenteral administration includes intramuscular, intravenous, intraarterial, intraperitoneal, or subcutaneous administration. The compositions utilized in the methods described herein can also be administered systemically or locally. The method of administration can vary depending on various factors (e.g., the compound or composition being administered and the severity of the condition, disease, or disorder being treated).

The terms “therapeutic agent” or “agent” refer to any agent that is used to treat a disease, e.g., a mast cell-mediated inflammatory disease, e.g., asthma. A therapeutic agent may be, for example, a polypeptide(s) (e.g., an antibody, an immunoadhesin, or a peptibody), an aptamer, a small molecule that can bind to a protein, or a nucleic acid molecule that can bind to a nucleic acid molecule encoding a target (e.g., siRNA), and the like.

The terms “inhibitors” and “antagonists,” as used interchangeably herein, refer to compounds or agents which inhibit or reduce the biological activity of the molecule to which they bind. Inhibitors include antibodies, synthetic or native-sequence peptides, immunoadhesins, and small-molecule inhibitors that bind to, for example, tryptase or IgE. In certain embodiments, an inhibitor (e.g., an antibody) inhibits an activity of the antigen by at least 10% in the presence of the inhibitor compared to the activity in the absence of the inhibitor. In some embodiments, an inhibitor inhibits an activity by at least 20%, at least 30%, at least 40%, at least 50%, at least 60%, at least 70%, at least 80%, at least 90%, or 100%.

As used herein, the term “tryptase antagonist” refers to compounds or agents which inhibit or reduce the biological activity of tryptase (e.g., tryptase alpha (e.g., tryptase alpha I) or tryptase beta (e.g., tryptase beta I, tryptase beta II, or tryptase beta III)). In some embodiment, a tryptase antagonist is an anti-tryptase antibody or a small molecule inhibitor.

The terms “anti-tryptase antibody,” an “antibody that binds to tryptase,” and “antibody that specifically binds tryptase” refer to an antibody that is capable of binding tryptase with sufficient affinity such that the antibody is useful as a diagnostic and/or therapeutic agent in targeting tryptase. In one embodiment, the extent of binding of an anti-tryptase antibody to an unrelated, non-tryptase protein is less than about 10% of the binding of the antibody to tryptase as measured, e.g., by a radioimmunoassay (RIA). In certain embodiments, an antibody that binds to tryptase has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹M to 10⁻¹³ M). In certain embodiments, an anti-tryptase antibody binds to an epitope of tryptase that is conserved among tryptase from different species. Exemplary anti-tryptase antibodies are described herein and in U.S. Provisional Patent Application No. 62/457,722 and International Patent Application Publication No. WO 2018/148585, which are incorporated herein by reference in their entirety.

The term “FcεRI” refers to refers to any native FcεRI (also known in the art as high-affinity IgE receptor or FCER1) from any vertebrate source, including mammals such as primates (e.g., humans) and rodents (e.g., mice and rats), unless otherwise indicated. FcεRI is a tetrameric receptor complex that binds the Fc protein of the c heavy chain of IgE. FcεRI is composed of one α chain, one β chain, and two γ chains. The amino acid sequence of an exemplary human FcϵRIα polypeptide is listed under UniProt Accession No. P12319. The amino acid sequence of an exemplary human FcεRIβ polypeptide is listed under UniProt Accession No. 001362. The amino acid sequence of an exemplary human FcεRIγ polypeptide is listed under UniProt Accession No. P30273.

The term “FcεRII” refers to refers to any native FcεRII (also known in the art as CD23, FCER2, or low-affinity IgE receptor) from any vertebrate source, including mammals such as primates (e.g., humans) and rodents (e.g., mice and rats), unless otherwise indicated. The term encompasses “full-length,” unprocessed FcεRII as well as any form of FcεRII that results from processing in the cell. The term also encompasses naturally occurring variants of FcεRII, e.g., splice variants or allelic variants. The amino acid sequence of an exemplary human FcεRII polypeptide is listed under UniProt Accession No. P06734.

As used herein, the term “Fc epsilon receptor (FcεR) antagonist” refers to compounds or agents which inhibit or reduce the biological activity of FcεR (e.g., FcεRI or FcεRII). The FcεR antagonist may inhibit the activity of FcεR or a nucleic acid (e.g., a gene or mRNA transcribed from the gene) or polypeptide that is involved in FcεR signal transduction. For example, in some embodiments, the FcεR antagonist inhibits tyrosine-protein kinase Lyn (Lyn), Bruton's tyrosine kinase (BTK), tyrosine-protein kinase Fyn (Fyn), spleen associated tyrosine kinase (Syk), linker for activation of T cells (LAT), growth factor receptor bound protein 2 (Grb2), son of sevenless (Sos), Ras, Raf-1, mitogen-activated protein kinase kinase 1 (MEK), mitogen-activated protein kinase 1 (ERK), cytosolic phospholipase A2 (cPLA2), arachidonate 5-lipoxygenase (5-LO), arachidonate 5-lipoxygenase activating protein (FLAP), guanine nucleotide exchange factor VAV (Vav), Rac, mitogen-activated protein kinase kinase 3, mitogen-activated protein kinase kinase 7, p38 MAP kinase (p38), c-Jun N-terminal kinase (JNK), growth factor receptor bound protein 2-associated protein 2 (Gab2), phosphatidylinositol-4,5-bisphosphate 3-kinase (PI3K), phospholipase C gamma (PLCγ), protein kinase C (PKC), 3-phosphoinositide dependent protein kinase 1 (PDK1), RAC serine/threonine-protein kinase (AKT), histamine, heparin, interleukin (IL)-3, IL-4, IL-13, IL-5, granulocyte-macrophage colony-stimulating factor (GM-CSF), tumor necrosis factor alpha (TNFα), leukotrienes (e.g., LTC4, LTD4 and LTE4), and prostaglandins (e.g., PDG2). In some embodiments, the FcεR antagonist is a BTK inhibitor, e.g., GDC-0853, acalabrutinib, GS-4059, spebrutinib, BGB-3111, or HM71224.

A “B cell” is a lymphocyte that matures within the bone marrow, and includes a naïve B cell, memory B cell, or effector B cell (plasma cells). The B cell herein may be normal or non-malignant.

The term “IgE⁺ B cell depleting antibody” refers to an antibody that can reduce the number of IgE⁺ B cells in a subject and/or interfere with one or more IgE⁺ B cell functions. An “IgE⁺ B cell” refers to a B cell that expresses the membrane B cell receptor form of IgE. In some embodiments, the IgE⁺ B cell is an IgE-switched B cell or a memory B cell. Human membrane IgE contains an extracellular 52 amino acid segment referred to as M1 prime (also known as M1′, me.1, or CemX) that is not expressed in secreted IgE antibodies. In some embodiments, the IgE⁺ B cell depleting antibody is an anti-M1′ antibody (e.g., quilizumab). In some embodiments, the anti-M1′ antibody is any anti-M1′ antibody described in International Patent Application Publication No. WO 2008/116149.

A “mast cell” is a type of granulocyte immune cell. Mast cells are typically present in mucosal and epithelial tissues throughout the body. Mast cells contain cytoplasmic granules that store inflammatory mediators, including tryptase (particularly tryptase beta), histamine, heparin, and cytokines. Mast cells can be activated by antigen/IgE/FcεRI cross-linking, which can result in degranulation and release of inflammatory mediators. A mast cell may be a mucosal mast cell or a connective tissue mast cell. See, e.g., Krystel-Whittemore et al. Front. Immunol. 6:620, 2015.

A “basophil” is a type of granulocyte immune cell. Basophils are typically present in peripheral blood. Basophils can be activated via antigen/IgE/FcεRI cross-linking to release molecules such as histamines, tryptase (particularly tryptase alpha), leukotrienes, and cytokines. See, e.g., Siracusa et al. J. Allergy Clin. Immunol. 132(4):789-801, 2013.

The term “mast cell or basophil depleting antibody” refers to an antibody that can reduce the number or biological activity of mast cells or basophils in a subject and/or interfere with one or more functions of mast cells or basophils. In some embodiments, the antibody is a mast cell depleting antibody. In other embodiments, the antibody is a basophil depleting antibody. In yet other embodiments, the antibody depletes mast cells and basophils. In some embodiments, the mast cell or basophil depleting antibody is an anti-Siglec8 antibody.

The term “protease-activated receptor 2 (PAR2)” refers to refers to any native PAR2 (also known in the art as F2R like trypsin receptor 1 (F2RL1) or G-protein coupled receptor 11 (GPR11)) from any vertebrate source, including mammals such as primates (e.g., humans) and rodents (e.g., mice and rats), unless otherwise indicated. The term encompasses “full-length,” unprocessed PAR2 as well as any form of PAR2 that results from processing in the cell. The term also encompasses naturally occurring variants of PAR2, e.g., splice variants or allelic variants. The nucleic acid sequence of an exemplary human PAR2 is listed in RefSeq Accession No. NM_005252. The amino acid sequence of an exemplary protein encoded by human PAR2 is listed in UniProt Accession No. P55085.

The term “PAR2 antagonist” refers to a molecule that decreases, blocks, inhibits, abrogates, or interferes with PAR2 biological activity or signal transduction. PAR2 is typically activated by proteolytic cleavage of its N-terminus, which unmasks a tethered peptide ligand that binds and activates the transmembrane receptor domain. Exemplary PAR2 antagonists include small molecule inhibitors (e.g., K-12940, K-14585, GB83, GB88, AZ3451, and AZ8838), soluble receptors, siRNAs, and anti-PAR2 antibodies (e.g., MAB3949 and Fab3949). See, e.g., Cheng et al. Nature 545:112-115, 2017; Kanke et al. Br. J. Pharmacol. 158(1):361-371, 2009; and Lohman et al. FASEB J. 26(7):2877-2887, 2012.

The term “IgE antagonist” refers to a molecule that decreases, blocks, inhibits, abrogates, or interferes with IgE biological activity. Such antagonists include but are not limited to anti-IgE antibodies, IgE receptors, anti-IgE receptor antibodies, variants of IgE antibodies, ligands for the IgE receptors, and fragments thereof. In some embodiments, an IgE antagonist is capable of disrupting or blocking the interaction between IgE (e.g., human IgE) and the high affinity receptor FcεRI, for example, on mast cells or basophils.

An “anti-IgE antibody” includes any antibody that binds specifically to IgE in a manner so as to not induce cross-linking when IgE is bound to the high affinity receptor on mast cells and basophils. Exemplary anti-IgE antibodies include rhuMabE25 (E25, omalizumab (XOLAIR®)), E26, E27, as well as CGP-5101 (Hu-901), the HA antibody, ligelizumab, and talizumab. The amino acid sequences of the heavy and light chain variable domains of the humanized anti-IgE antibodies E25, E26 and E27 are disclosed, for example, in U.S. Pat. No. 6,172,213 and WO 99/01556. The CGP-5101 (Hu-901) antibody is described in Corne et al. J. Clin. Invest. 99(5): 879-887, 1997; WO 92/17207; and ATCC Dep. Nos. BRL-10706, BRL-11130, BRL-11131, BRL-11132 and BRL-11133. The HA antibody is described in U.S. Ser. No. 60/444,229, WO 2004/070011, and WO 2004/070010.

The term “interleukin-33 (IL-33),” as used herein, refers to any native IL-33 from any vertebrate source, including mammals such as primates (e.g., humans) and rodents (e.g., mice and rats), unless otherwise indicated. IL-33 is also referred to in the art as nuclear factor of high endothelial venules (NF-HEV; see, e.g., Baekkevold et al. Am. J. Pathol. 163(1): 69-79, 2003), DVS27, C9orf26, and interleukin-1 family member 11 (IL-1F11). The term encompasses “full-length,” unprocessed IL-33, as well as any form of IL-33 that results from processing in the cell. Human full-length, unprocessed IL-33 contains 270 amino acids (a.a.) and may also be referred to as IL-331-270. Processed forms of human IL-33 include, for example, IL-33₉₅₋₂₇₀, IL-33₉₉₋₂₇₀, IL-33₁₀₉₋₂₇₀, IL-33₁₁₂₋₂₇₀, IL-33₁₋₁₇₈, and IL-33₁₇₉₋₂₇₀ (Lefrançais et al. Proc. Natl. Acad. Sci. 109(5): 1673-1678, 2012 and Martin, Semin. Immunol. 25: 449-457, 2013). In some embodiments, processed forms of human IL-33, e.g., IL-3395-270, IL-3399-270, IL-33109-270, or other forms processed by proteases such as calpain, proteinase 3, neutrophil elastase, and cathepsin G may have increased biological activity compared to full-length IL-33. The term also encompasses naturally occurring variants of IL-33, for example, splice variants (e.g., the constitutively active splice variant spIL-33 which lacks exon 3, Hong et al. J. Biol. Chem. 286(22): 20078-20086, 2011) or allelic variants. IL-33 may be present within a cell (e.g., within the nucleus) or as a secreted cytokine form. Full-length IL-33 protein contains a helix-turn-helix DNA-binding motif including nuclear localization sequence (a.a. 1-75 of human IL-33), which includes a chromatin binding motif (a.a. 40-58 of human IL-33). Forms of IL-33 that are processed and secreted lack these N-terminal motifs. The amino acid sequence of an exemplary human IL-33 can be found, for example, under UniProt accession number 095760.

By “IL-33 axis” is meant a nucleic acid (e.g., a gene or mRNA transcribed from the gene) or polypeptide that is involved in IL-33 signal transduction. For example, the IL-33 axis may include the ligand IL-33, a receptor (e.g., ST2 and/or IL-1 RAcP), adaptor molecules (e.g., MyD88), or proteins that associate with receptor molecules and/or adaptor molecules (e.g., kinases, such as interleukin-1 receptor-associated kinase 1 (IRAK1) and interleukin-1 receptor-associated kinase 4 (IRAK4), or E3 ubiquitin ligases, such as TNF receptor associated factor 6 (TRAF6)).

An “IL-33 axis binding antagonist” refers to a molecule that inhibits the interaction of an IL-33 axis binding partner with one or more of its binding partners. As used herein, an IL-33 axis binding antagonist includes IL-33 binding antagonists, ST2 binding antagonists, and IL1 RAcP binding antagonists. Exemplary IL-33 axis binding antagonists include anti-IL-33 antibodies and antigen-binding fragments thereof (e.g., anti-IL-33 antibodies such as ANB-020 (AnaptysBio Inc.) or any of the antibodies described in EP1725261, U.S. Pat. No. 8,187,596, WO 2011/031600, WO 2014/164959, WO 2015/099175, WO 2015/106080, or WO 2016/077381, which are each incorporated herein by reference in their entirety); polypeptides that bind IL-33 and/or its receptor (ST2 and/or IL-1 RAcP) and block ligand-receptor interaction (e.g., ST2-Fc proteins; immunoadhesins, peptibodies, and soluble ST2, or derivatives thereof); anti-IL-33 receptor antibodies (e.g., anti-ST2 antibodies, for example, AMG-282 (Amgen) or STLM15 (Janssen) or any of the anti-ST2 antibodies described in WO 2013/173761 or WO 2013/165894, which are each incorporated herein by reference in their entirety; or ST2-Fc proteins, such as those described in WO 2013/173761; WO 2013/165894; or WO 2014/152195, which are each incorporated herein by reference in their entirety); and IL-33 receptor antagonists, such as small molecule inhibitors, aptamers that bind IL-33, and nucleic acids that hybridize under stringent conditions to IL-33 axis nucleic acid sequences (e.g., short interfering RNAs (siRNA) or clustered regularly interspaced short palindromic repeat RNAs (CRISPR-RNA or crRNA)).

The term “ST2 binding antagonist” refers to a molecule that inhibits the interaction of an ST2 with IL-33, IL1 RAcP, and/or a second ST2 molecule. The ST2 binding antagonist may be a protein, such as an “ST2-Fc protein” that includes an IL-33-binding domain (e.g., all or a portion of an ST2 or IL1 RAcP protein) and a multimerizing domain (e.g., an Fc portion of an immunoglobulin, e.g., an Fc domain of an IgG selected from the isotypes IgG1, IgG2, IgG3, and IgG4, as well as any allotype within each isotype group), which are attached to one another either directly or indirectly through a linker (e.g., a serine-glycine (SG) linker, glycine-glycine (GG) linker, or variant thereof (e.g., a SGG, a GGS, an SGS, or a GSG linker)), and includes, but is not limited to, ST2-Fc proteins and variants thereof described in WO 2013/173761, WO 2013/165894, and WO 2014/152195, which are each incorporated herein by reference in their entirety.

A “T_{H}2 pathway inhibitor” or “T_{H}2 inhibitor” is an agent that inhibits the T_{H}2 pathway. Examples of a T_{H}2 pathway inhibitor include inhibitors of the activity of any one of the targets selected from interleukin-2-inducible T cell kinase (ITK), Bruton's tyrosine kinase (BTK), Janus kinase 1 (JAK1) (e.g., ruxolitinib, tofacitinib, oclacitinib, baricitinib, filgotinib, gandotinib, lestaurtinib, momelotinib, pacrinitib, upadacitinib, peficitinib, and fedratinib), GATA binding protein 3 (GATA3), IL-9 (e.g., MEDI-528), IL-5 (e.g., mepolizumab, CAS No. 196078-29-2; resilizumab), IL-13 (e.g., IMA-026, IMA-638 (also referred to as anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody)), IL-4 (e.g., AER-001, IL-4/IL-13 trap), OX40L, TSLP, IL-25, IL-33, and IgE (e.g., XOLAIR®, QGE-031; and MEDI-4212); and receptors such as: IL-9 receptor, IL-5 receptor (e.g., MEDI-563 (benralizumab, CAS No. 1044511-01-4)), IL-4 receptor alpha (e.g., AMG-317, AIR-645), IL-13 receptoralpha1 (e.g., R-1671) and IL-13 receptoralpha2, OX40, TSLP-R, IL-7Ralpha (a co-receptor for TSLP), IL-17RB (receptor for IL-25), ST2 (receptor for IL-33), CCR3, CCR4, CRTH2 (e.g., AMG-853, AP768, AP-761, MLN6095, ACT129968), FcεRI, FcεRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL-5, IL-3, and GM-CSF (e.g., TPI ASM8). Examples of inhibitors of the aforementioned targets are disclosed in, for example, WO 2008/086395; WO 2006/085938; U.S. Pat. Nos. 7,615,213; 7,501,121; WO 2006/085938; WO 2007/080174; U.S. Pat. No. 7,807,788; WO 2005/007699; WO 2007/036745; WO 2009/009775; WO 2007/082068; WO 2010/073119; WO 2007/045477; WO 2008/134724; US 2009/0047277; and WO 2008/127271.

The terms “patient” or “subject” refer to any single animal, more specifically a mammal (including such non-human animals as, for example, cats, dogs, horses, rabbits, cows, pigs, sheep, zoo animals, and non-human primates) for which diagnosis or treatment is desired. Even more specifically, the patient herein is a human.

The term “small molecule” refers to an organic molecule having a molecular weight between 50 Daltons to 2500 Daltons.

The term “effective amount” refers to an amount of a drug or therapeutic agent (e.g., a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist)) effective to treat a disease or disorder (e.g., a mast cell-mediated inflammatory disease, e.g., asthma) in a subject or patient, such as a mammal, e.g., a human.

As used herein, “therapy” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. Those in need of treatment include can include those already with the disorder as well as those at risk to have the disorder or those in whom the disorder is to be prevented. A patient may be successfully “treated” for asthma if, for example, after receiving an asthma therapy, the patient shows observable and/or measurable reduction in or absence of one or more of the following: recurrent wheezing, coughing, trouble breathing, chest tightness, symptoms that occur or worsen at night, symptoms that are triggered by cold air, exercise or exposure to allergens.

A “response” of a patient or a patient's “responsiveness” to treatment or therapy, for example a therapy including a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist), refers to the clinical or therapeutic benefit imparted to a patient at risk for or having asthma from or as a result of the treatment. A skilled person will readily be in position to determine whether a patient is responsive. For example, a patient having asthma who is responsive to a therapy including a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist) may show observable and/or measurable reduction in or absence of one or more asthma symptoms, for example, recurrent wheezing, coughing, trouble breathing, chest tightness, symptoms that occur or worsen at night, symptoms that are triggered by cold air, exercise or exposure to allergens. In some embodiments, a response may be an improvement in lung function, e.g., an improvement in FEN/1%.

The terms “sample” and “biological sample” are used interchangeably to refer to any biological sample derived from an individual including body fluids, body tissue (e.g., lung samples), nasal samples (including nasal swabs or nasal polyps), sputum, nasosorption samples, bronchosorption samples, cells, or other sources. Body fluids include, e.g., bronchiolar lavage fluid (BAL), mucosal lining fluid (MLF; including, e.g., nasal MLF or bronchial MLF), lymph, sera, whole fresh blood, frozen whole blood, plasma (including fresh or frozen), serum (including fresh or frozen), peripheral blood mononuclear cells, urine, saliva, semen, synovial fluid, and spinal fluid. Methods for obtaining tissue biopsies and body fluids from mammals are well known in the art.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “affinity-matured” antibody is one with one or more alterations in one or more HVRs and/or framework regions which result in an improvement in the affinity of the antibody for antigen, compared to a parent antibody which does not possess those alteration(s). Preferred affinity-matured antibodies will have nanomolar or even picomolar affinities for the target antigen. Affinity-matured antibodies are produced by procedures known in the art. For example, Marks et al. Bio/Technology 10:779-783, 1992 describes affinity maturation by VH and VL domain shuffling. Random mutagenesis of HVR and/or framework residues is described by: Barbas et al. Proc. Natl. Acad. Sci. USA 91:3809-3813, 1994; Schier et al. Gene 169:147-155, 1995; Yelton et al. J. Immunol. 155:1994-2004, 1995; Jackson et al. J. Immunol. 154(7):3310-3319, 1995; and Hawkins et al. J. Mol. Biol. 226:889-896, 1992.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more, 60% or more, 70% or more, 80% or more, or 90% or more. In some embodiments, the set of amino acid residues contacted by the antibody may be completely overlapping or partially overlapping with the set of amino acid residues contacted by the reference antibody. In some embodiments, an antibody that binds to the same epitope as a reference antibody blocks binding of the reference antibody to its antigen in a competition assay by 50% or more, 60% or more, 70% or more, 80% or more, or 90% or more, and conversely, the reference antibody blocks binding of the antibody to its antigen in a competition assay by 50% or more, 60% or more, 70% or more, 80% or more, or 90% or more. An exemplary competition assay is provided herein.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al. Protein Eng. 8(10):1057-1062, 1995); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, Md., 1991.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three Hs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315, 1994.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al. Proc. Natl. Acad. Sci. USA 90:6444-6448, 1993.

A “blocking” antibody or an “antagonist” antibody is one which inhibits or reduces biological activity of the antigen it binds. Certain blocking antibodies or antagonist antibodies substantially or completely inhibit the biological activity of the antigen. For example, with respect to anti-tryptase antibodies, in some embodiments, the activity may be a tryptase enzymatic activity, e.g., protease activity. In other instances, the activity may be tryptase-mediated stimulation of bronchial smooth muscle cell proliferation and/or collagen-based contraction. In other instances, the activity may be mast cell histamine release (e.g., IgE-triggered histamine release and/or tryptase-triggered histamine release). In some embodiments, an antibody can inhibit a biological activity of the antigen it binds by at least about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch et al. Annu. Rev. Immunol. 9:457-492, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA 95:652-656, 1998.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Da\tilde{e}ron, Annu. Rev. Immunol. 15:203-234, 1997). FcRs are reviewed, for example, in Ravetch et al. Annu. Rev. Immunol. 9:457-492, 1991; Capel et al. Immunomethods 4:25-34, 1994; and de Haas et al. J. Lab. Clin. Med. 126:330-41, 1995. Other FcRs, including those to be identified in the future, are encompassed by the term “FcR” herein. The term also includes the neonatal receptor, FcRn, which is responsible for the transfer of maternal IgGs to the fetus (see, e.g., Guyer et al. J. Immunol. 117:587, 1976; and Kim et al. J. Immunol. 24:249, 1994).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al. J. Immunol. Methods 202:163, 1996, can be performed.

An “epitope” is the portion of the antigen to which the antibody selectively binds. For a polypeptide antigen, a linear epitope can be a peptide portion of about 4-15 (e.g., 4, 5, 6, 7, 8, 9, 10, 11, 12, amino acid residues. A non-linear, conformational epitope may comprise residues of a polypeptide sequence brought to close vicinity in the three-dimensional (3D) structure of the protein. In some embodiments, the epitope comprises amino acids that are within 4 angstroms (Å) of any atom of an antibody. In certain embodiments, the epitope comprises amino acids that are within 3.5 Å, 3 Å, 2.5 Λ, or 2 Å of any atom of an antibody. The amino acid residues of an antibody that contact an antigen (i.e., paratope) can be determined, for example, by determining the crystal structure of the antibody in complex with the antigen or by performing hydrogen/deuterium exchange.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human and/or has been made using any of the techniques for making human antibodies. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda Md., vols. 1-3, 1991. In one embodiment, for the VL, the subgroup is subgroup kappa III or kappa IV as in Kabat et al. supra. In one embodiment, for the VH, the subgroup is subgroup III as in Kabat et al. supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525, 1986; Riechmann et al. Nature 332:323-329, 1988; and Presta, Curr. Op. Struct. Biol. 2:593-596, 1992.

An “immunoconjugate” is an antibody conjugated to one or more heterologous molecule(s), including but not limited to a cytotoxic agent.

The term “isolated” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., sodium dodecyl sulfate polyacrylamide gel electrophoresis (SDS-PAGE), isoelectric focusing (IEF), capillary electrophoresis) or chromatographic (e.g., ion exchange or reverse phase HPLC) methods. For a review of methods for assessment of antibody purity, see, for example, Flatman et al. J. Chromatogr. B 848:79-87, 2007. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope on an antigen, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In certain embodiments, the term “monoclonal antibody” encompasses bispecific antibodies.

The term “bivalent antibody” refers to an antibody that has two binding sites for the antigen. A bivalent antibody can be, without limitation, in the IgG format or in the F(ab′)2 format.

The term “multispecific antibody” is used in the broadest sense and covers an antibody that binds to two or more determinants or epitopes on one antigen or two or more determinants or epitopes on more than one antigen. Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). In certain embodiments, the multispecific antibody is a bispecific antibody. “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody binds to each epitope with an affinity of 5 μM to 0.001 pM, 3 μM to 0.001 pM, 1 μM to 0.001 pM, 0.5 μM to 0.001 pM or 0.1 pM to 0.001 pM. “Monospecific” refers to the ability to bind only one epitope.

A “naked antibody” refers to an antibody that is not conjugated to a heterologous moiety (e.g., a cytotoxic moiety) or radiolabel. The naked antibody may be present in a pharmaceutical composition.

With regard to the binding of a antibody to a target molecule, the term “binds” or “binding” or “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The term “variable” refers to the fact that certain segments of the variable domains differ extensively in sequence among antibodies. The variable or “V” domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The term “hypervariable region” or “HVR” when used herein refers to the amino acid residues of an antibody which are responsible for antigen-binding. The hypervariable region generally comprises amino acid residues from e.g., around about residues 24-34 (L1), 50-56 (L2) and 89-97 (L3) in the VL, and around about residues 26-35 (H1), 49-65 (H2) and 95-102 (H3) in the VH (in one embodiment, H1 is around about residues 31-35); Kabat et al. supra) and/or those residues from a “hypervariable loop” (e.g., residues 26-32 (L1), 50-52 (L2), and 91-96 (L3) in the VL, and 26-32 (H1), 53-55 (H2), and 96-101 (H3) in the VH; Chothia et al. J. Mol. Biol. 196:901-917, 1987. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al. supra). Accordingly, the HVR and FR sequences generally appear in the following sequence in VH (or VL): FR1-H1(L1)-FR2-H2(L2)-FR3-H3(L3)-FR4. The constant domains are not involved directly in binding an antibody to an antigen, but exhibit various effector functions, such as participation of the antibody in antibody dependent cellular cytotoxicity (ADCC).

The term “variable domain residue numbering as in Kabat” or “amino acid position numbering as in Kabat,” and variations thereof, refers to the numbering system used for heavy chain variable domains or light chain variable domains of the compilation of antibodies in Kabat et al. supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al. supra). The “EU numbering system” or “EU index” is generally used when referring to a residue in an immunoglobulin heavy chain constant region (e.g., the EU index reported in Kabat et al. supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

By “massively parallel sequencing” or “massive parallel sequencing,” also known in the art as “next-generation sequencing,” or “second generation sequencing,” is meant any high-throughput nucleic acid sequencing approach. These approaches typically involve parallel sequencing of a large number (e.g., thousands, millions, or billions) of spatially separated, clonally amplified DNA templates or single DNA molecules. See, for example, Metzker, Nature Reviews Genetics 11: 31-36, 2010.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “pharmaceutical formulation” and “pharmaceutical composition” are used interchangeably herein, and refer to a preparation which is in such form as to permit the biological activity of an active ingredient contained therein to be effective, and which contains no additional components which are unacceptably toxic to a subject to which the formulation would be administered. Such formulations are sterile.

A “sterile” pharmaceutical formulation is aseptic or free or essentially free from all living microorganisms and their spores.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “kit” is any manufacture (e.g., a package or container) comprising at least one reagent, for example, a probe for determining a patient's active tryptase allele count or for determining the expression level of a biomarker (e.g., tryptase) as described herein and/or a medicament for treatment of a mast cell-mediated inflammatory disease, e.g., asthma. The manufacture is preferably promoted, distributed, or sold as a unit for performing the methods of the present invention.

### II. Therapeutic Methods and Uses of the Invention

The present invention features methods of treating a patient having a mast cell-mediated inflammatory disease (e.g., asthma). In some embodiments, the methods of the invention include administering a therapy to a patient based on the presence and/or expression level of a biomarker of the invention, for example, tryptase (e.g., the patient's active tryptase allele count and/or the expression level of tryptase). In some embodiments, the methods involve administering a therapy, for example, a therapy including a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, or a combination thereof (e.g., a tryptase antagonist and an IgE antagonist). In some embodiments, the therapy includes a mast-cell directed therapy (e.g. a tryptase antagonist, an IgE antagonist, an IgE⁺ B cell depleting antibody, a mast cell or basophil depleting antibody, and/or a PAR2 antagonist). In some embodiments, the therapy includes a tryptase antagonist (e.g., an anti-tryptase antibody, e.g., any anti-tryptase antibody described herein or in WO 2018/148585) and an IgE antagonist (e.g., an anti-IgE antibody, e.g., omalizumab (XOLAIR®)).

For example, the invention features a method of treating a patient having a mast cell-mediated inflammatory disease that includes administering to a patient having a mast cell-mediated inflammatory disease a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)), wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. For example, in some embodiments, the genotype of the patient has been determined to comprise an active tryptase allele count that is at or above a reference active tryptase allele count. In other embodiments, a sample from the patient has been determined to have an expression level of tryptase that is at or above a reference level of tryptase.

In another aspect, the invention features a method of treating a patient having a mast cell-mediated inflammatory disease who has been identified as having (i) a genotype comprising an active tryptase allele count that is at or above a reference active tryptase allele count; or (ii) an expression level of tryptase in a sample from the patient that is at or above a reference level of tryptase, the method including administering to a patient having a mast cell-mediated inflammatory disease a mast-cell directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)). For example, in some embodiments, the genotype of the patient has been idendified to comprise an active tryptase allele count that is at or above a reference active tryptase allele count. In other embodiments, the patient has been identified to have an expression level of tryptase in a sample from the patient that is at or above a reference level of tryptase.

In another aspect, the invention features a method of treating a patient having a mast cell-mediated inflammatory disease, the method including: (a) obtaining a sample containing a nucleic acid from the patient; (b) performing a genotyping on the sample and detecting the presence of an active tryptase allele count that is at or above a reference level of tryptase; (c) identifying the patient having the active tryptase allele count that is at or above a reference level of tryptase as having an increased likelihood of benefiting from treatment with a mast cell-directed therapy (e.g., a therapy comprising a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)); and (d) administering a mast-cell directed therapy (e.g., a therapy comprising a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) to the patient.

In a still further aspect, the invention features a method of treating a patient having a mast cell-mediated inflammatory disease, the method including: (a) obtaining a sample containing a nucleic acid or protein from the patient; (b) performing an expression assay and detecting an expression level of tryptase that is at or above a reference level of tryptase; (c) identifying the patient having an expression level of tryptase that is at or above a reference level of tryptase as having an increased likelihood of benefiting from treatment with a mast cell-directed therapy (e.g., a therapy comprising a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)); and (d) administering a mast-cell-directed therapy (e.g., a therapy comprising a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) to the patient. In some embodiments, the sample contains a protein and the expression assay is an ELISA or an immunoassay.

In some embodiments of any of the preceding methods, the patient has been identified as having a level of a Type 2 biomarker in a sample from the patient that is below a reference level of the Type 2 biomarker. In some embodiments, the agent is administered to the patient as a monotherapy.

In some embodiments of any of the preceding methods, the patient has been identified as having a level of a Type 2 biomarker in a sample from the patient that is at or above a reference level of the Type 2 biomarker. In some embodiments, the method further comprises administering a T_{H}2 pathway inhibitor to the patient.

In another aspect, the invention features a method of treating a patient having a mast cell-mediated inflammatory disease that includes administering to a patient having a mast cell-mediated inflammatory disease a therapy comprising an IgE antagonist or a FcεR antagonist, wherein (i) the genotype of the patient has been determined to comprise an active tryptase allele count that is below a reference active tryptase allele count; or (ii) a sample from the patient has been determined to have an expression level of tryptase that is below a reference level of tryptase. For example, in some embodiments, the genotype of the patient has been determined to comprise an active tryptase allele count that is below a reference active tryptase allele count. In other embodiments, a sample from the patient has been determined to have an expression level of tryptase that is below a reference level of tryptase.

In another aspect, the invention features a method of treating a patient having a mast cell-mediated inflammatory disease who has been identified as having (i) a genotype comprising an active tryptase allele count that is below a reference active tryptase allele count; or (ii) an expression level of tryptase in a sample from the patient that is below a reference level of tryptase, the method including administering to a patient having a mast cell-mediated inflammatory disease a therapy comprising an IgE antagonist or a FcεR antagonist. For example, in some embodiments, the genotype of the patient has been identified to comprise an active tryptase allele count that is below a reference active tryptase allele count. In other embodiments the patient has been identified to have an expression level of tryptase in a sample from the patient that is below a reference level of tryptase.

In another aspect, the invention features a method of treating a patient having a mast cell-mediated inflammatory disease, the method including: (a) obtaining a sample containing a nucleic acid from the patient; (b) performing a genotyping on the sample and detecting the presence of an active tryptase allele count that is below a reference level of tryptase; (c) identifying the patient having the active tryptase allele count that is below a reference level of tryptase as having an increased likelihood of benefiting from treatment with an IgE antagonist or a FcεR antagonist; and (d) administering an IgE antagonist or a FcεR antagonist to the patient.

In a still further aspect, the invention features a method of treating a patient having a mast cell-mediated inflammatory disease, the method including: (a) obtaining a sample containing a nucleic acid or protein from the patient; (b) performing an expression assay and detecting an expression level of tryptase that is below a reference level of tryptase; (c) identifying the patient having an expression level of tryptase that is below a reference level of tryptase as having an increased likelihood of benefiting from treatment with an IgE antagonist or a FcεR antagonist; and (d) administering an IgE antagonist or a FcεR antagonist to the patient. In some embodiments, the sample contains a protein and the expression assay is an ELISA or an immunoassay.

In some embodiments of any of the preceding methods, the patient has been identified as having a level of a Type 2 biomarker in a sample from the patient that is at or above a reference level of the Type 2 biomarker. In some embodiments, the method further comprises administering an additional T_{H}2 pathway inhibitor to the patient.

In some embodiments of any of the preceding methods, the active tryptase allele count has been determined by sequencing the TPSAB1 and TPSB2 loci of the patient's genome. Any suitable sequencing approach can be used, for example, Sanger sequencing or massively parallel (e.g., ILLUMINA®) sequencing. In some embodiments, the TPSAB1 locus is sequenced by a method comprising (i) amplifying a nucleic acid from the subject in the presence of a first forward primer comprising the nucleotide sequence of 5′-CTG GTG TGC AAG GTG AAT GG-3′ (SEQ ID NO: 31) and a first reverse primer comprising the nucleotide sequence of 5′-AGG TOO AGO ACT CAG GAG GA-3′ (SEQ ID NO: 32) to form a TPSAB1 amplicon, and (ii) sequencing the TPSAB1 amplicon. In some embodiments, sequencing the TPSAB1 amplicon comprises using the first forward primer and the first reverse primer. In some embodiments, the TPSB2 locus is sequenced by a method comprising (i) amplifying a nucleic acid from the subject in the presence of a second forward primer comprising the nucleotide sequence of 5′-GCA GGT GAG COT GAG AGT CC-3′ (SEQ ID NO: 33) and a second reverse primer comprising the nucleotide sequence of 5′-GGG ACC TTC ACC TOO TTC AG-3′ (SEQ ID NO: 34) to form a TPSB2 amplicon, and (ii) sequencing the TPSB2 amplicon. In some embodiments, sequencing the TPSB2 amplicon comprises Lasing the second forward primer and a sequencing reverse primer comprising the nucleotide sequence of 5′-CAG CCA GTG ACC CAG CAC-3′ (SEQ ID NO: 35). In some embodiments, the active tryptase allele count may be determined by determining the presence of any variation in the TPSAB1 and TPSB2 loci of the patient's genome. In some embodiments, the active tryptase allele count is determined by the formula: 4—the sum of the number of tryptase alpha and tryptase beta III frame-shift (beta III^{FS}) alleles in the patient's genotype. In some embodiments, tryptase alpha is detected by detecting the c733 G>A SNP at TPSAB1. In some embodiments, detecting the c733 G>A SNP at TPSAB1 comprises detecting the patient's genotype at the polymorphism CTGCAGGCGGGCGTGGTCAGCTGGG[G/A]CGAGGGCTGTGCCCAGCCCAACCGG (SEQ ID NO: 36), wherein the presence of an A at the c733 G>A SNP indicates tryptase alpha. In some embodiments, tryptase beta III^{FS} is detected by detecting a c980_981insC mutation at TPSB2. In some embodiments, detecting a c980_981insC mutation at TPSB2 comprises detecting the nucleotide sequence CACACGGTCACCCTGCCCCCTGCCTCAGAGACCTTCCCCCCC (SEQ ID NO: 37).

In some embodiments of any of the preceding methods, the patient has an active tryptase allele count of 3 or 4. In some embodiments, the active tryptase allele count is 3. In other embodiments, the active tryptase allele count is 4.

In other embodiments of any of the preceding methods, the patient has an active tryptase allele count of 0, 1, or 2. In some embodiments, the active tryptase allele count is 0. In some embodiments, the active tryptase allele count is 1. In other embodiments, the active tryptase allele count is 2.

In some embodiments of any of the preceding methods, the reference active tryptase allele count can be determined in a reference sample, a reference population, and/or be a pre-assigned value (e.g., a cut-off value which was previously determined to significantly (e.g., statistically significantly) separate a first subset of individuals from a second subset of individuals (e.g., in terms of response to a therapy (e.g., a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof (e.g., a tryptase antagonist and an IgE antagonist))). In some embodiments, the reference active tryptase allele count is a pre-determined value. In some embodiments, the reference active tryptase allele count is predetermined in the mast cell-mediated inflammatory disease to which the patient belongs (e.g., asthma). In certain embodiments, the active tryptase allele count is determined from the overall distribution of the values in a mast cell-mediated inflammatory disease (e.g., asthma) investigated or in a given population. In some embodiments, a reference active tryptase allele count is an integer in the range of from 0 to 4 (e.g., 0, 1, 2, 3, or 4). In particular embodiments, a reference active tryptase allele count is 3.

In any of the preceding methods, the genotype of a patient can be determined using any of the methods or assays described herein (e.g., in Section IV of the Detailed Description of the Invention or in Example 1) or that are known in the art.

In some embodiments of any of the preceding aspects, the Type 2 biomarker is a T_{H}2 cell-related cytokine, periostin, eosinophil count, an eosinophil signature, FeNO, or IgE. In some embodiments, the T_{H}2 cell-related cytokine is IL-13, IL-4, IL-9, or IL-5.

In some embodiments of any of the preceding methods, the expression level of the biomarker (e.g., tryptase) is a protein expression level. For example, in some embodiments, the protein expression level has been measured using an immunoassay (e.g., a multiplexed immunoassay), ELISA, Western blot, or mass spectrometry. See, e.g., Section V of the Detailed Description of the Invention. In some embodiments, the protein expression level of tryptase is an expression level of active tryptase. In other embodiments, the protein expression level of tryptase is an expression level of total tryptase.

In other embodiments of any of the preceding methods, the expression level of the biomarker (e.g., tryptase) is an mRNA expression level. For example, in some embodiments, the mRNA expression level has been measured using a PCR method (e.g., qPCR) or a microarray chip. See, e.g., Section V of the Detailed Description of the Invention.

In any of the preceding methods or uses, the expression level of a biomarker of the invention (e.g., tryptase) in a sample derived from the patient may be changed at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker. For instance, in some embodiments, the expression level of a biomarker of the invention in a sample derived from the patient may be increased at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker. In other embodiments, the expression level of a biomarker of the invention in a sample derived from the patient may be decreased at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker.

In some embodiments, the reference level may be set to any percentile between, for example, the 20^{th} percentile and the 99^{th} percentile (e.g., the 20^{th}, 25^{th}, 30^{th}, 35^{th}, 40^{th},45^{th}, 50^{th}, 55^{th}, 60^{th}, 65^{th}, 70^{th}, 75^{th}, 80^{th}, 85^{th}, 90^{th}, 95^{th}, or 99^{th} percentile) of the overall distribution of the expression level of a biomarker (e.g., tryptase), for example, in healthy subjects or in a group of patients having a disorder (e.g., a mast cell-mediated inflammatory disease (e.g., asthma)). In particular embodiments, the reference level may be set to the 25^{th} percentile of the overall distribution of the values in a population of asthma patients. In other particular embodiments, the reference level may be set to the 50th percentile of the overall distribution of the values in a population of patients having asthma. In other embodiments, the reference level may be the median of the overall distribution of the values in a population of patients having asthma.

Any suitable sample derived from the patient may be used in any of the preceding methods. For example, in some embodiments, the sample derived from the patient is a blood sample (e.g., a whole blood sample, a serum sample, a plasma sample, or a combination thereof), a tissue sample, a sputum sample, a bronchiolar lavage sample, a mucosal lining fluid (MLF) sample, a bronchosorption sample, or a nasosorption sample.

The invention also features a mast-cell directed therapy (e.g., an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) for use in a method of treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor.

In another aspect, the invention provides for the use of a mast-cell directed therapy (e.g., an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) in the manufacture of a medicament for treating a patient having a mast cell-mediated inflammatory disease, wherein (i) the genotype of the patient has been determined to comprise an active tryptase allele count that is at or above a reference active tryptase allele count; or (ii) a sample from the patient has been determined to have an expression level of tryptase that is at or above a reference level of tryptase. In some embodiments, the patient has been determined to have a level of a Type 2 biomarker in a sample from the patient that is below a reference level of the Type 2 biomarker, and the agent is for use as a monotherapy. In some embodiments, the patient has been identified as having a level of a Type 2 biomarker in a sample from the patient that is at or above a reference level of the Type 2 biomarker, and the agent is for use in combination with a T_{H}2 pathway inhibitor.

In yet another aspect, the invention features an IgE antagonist or an FcεR antagonist for use in a method of treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with a T_{H}2 pathway inhibitor.

In a further aspect, the invention provides for the use of an IgE antagonist or an FcεR antagonist in the manufacture of a medicament for treating a patient having a mast cell-mediated inflammatory disease, wherein (i) the genotype of the patient has been determined to comprise an active tryptase allele count that is below a reference active tryptase allele count; or (ii) a sample from the patient has been determined to have an expression level of tryptase that is below a reference level of tryptase. In some embodiments, the patient has been determined to have a level of a Type 2 biomarker in a sample from the patient that is at or above a reference level of the Type 2 biomarker, and the IgE antagonist or FcεR antagonist is for use in combination with a T_{H}2 pathway inhibitor.

Any of the preceding methods or uses may include administering a tryptase antagonist to the patient. The tryptase antagonist may be a tryptase alpha antagonist (e.g., a tryptase alpha 1 antagonist) or a tryptase beta antagonist (e.g., a tryptase beta 1, tryptase beta 2, and/or tryptase beta 3 antagonist). In some embodiments, the tryptase antagonist is a tryptase alpha antagonist and a tryptase beta antagonist. In some embodiments, the tryptase antagonist (e.g., the tryptase alpha antagonist and/or the tryptase beta antagonist) is an anti-tryptase antibody (e.g., an anti-tryptase alpha antibody and/or an anti-tryptase beta antibody). Any anti-tryptase antibody described in Section VII below can be used.

Any of the preceding methods or uses may include administering an FcεR antagonist to the patient. In some embodiments, the FcεR antagonist inhibits FcεRIα, FcεRIβ, and/or FcεRIγ. In other embodiments, the FcεR antagonist inhibits FcεRII. In yet other embodiments, the FcεR antagonist inhibits a member of the FcεR signaling pathway. For example, in some embodiments, the FcεR antagonist inhibits tyrosine-protein kinase Lyn (Lyn), Bruton's tyrosine kinase (BTK), tyrosine-protein kinase Fyn (Fyn), spleen associated tyrosine kinase (Syk), linker for activation of T cells (LAT), growth factor receptor bound protein 2 (Grb2), son of sevenless (Sos), Ras, Raf-1, mitogen-activated protein kinase kinase 1 (MEK), mitogen-activated protein kinase 1 (ERK), cytosolic phospholipase A2 (cPLA2), arachidonate 5-lipoxygenase (5-LO), arachidonate 5-lipoxygenase activating protein (FLAP), guanine nucleotide exchange factor VAV (Vav), Rac, mitogen-activated protein kinase kinase 3, mitogen-activated protein kinase kinase 7, p38 MAP kinase (p38), c-Jun N-terminal kinase (JNK), growth factor receptor bound protein 2-associated protein 2 (Gab2), phosphatidylinositol-4,5-bisphosphate 3-kinase (PI3K), phospholipase C gamma (PLCγ), protein kinase C (PKC), 3-phosphoinositide dependent protein kinase 1 (PDK1), RAC serine/threonine-protein kinase (AKT), histamine, heparin, interleukin (IL)-3, IL-4, IL-13, IL-5, granulocyte-macrophage colony-stimulating factor (GM-CSF), tumor necrosis factor alpha (TNFα), leukotrienes (e.g., LTC4, LTD4 and LTE4) and prostaglandins (e.g., PDG2). In some embodiments, the FcεR antagonist is a BTK inhibitor (e.g., GDC-0853, acalabrutinib, GS-4059, spebrutinib, BGB-3111, or HM71224).

Any of the preceding methods or uses may include administering an IgE⁺ B cell depleting agent (e.g., an IgE⁺ B cell depleting antibody) to the patient. In some embodiments, the IgE⁺ B cell depleting antibody is an anti-M1′ domain antibody. Any suitable anti-M1′ domain antibody may be used, for example, any anti-M1′ domain antibody described in International Patent Application Publication No. WO 2008/116149, which is incorporated herein by reference in its entirety. In some embodiments, the anti-M1′ domain antibody is afucosylated. In some embodiments, the anti-M1′ domain antibody is quilizumab or 47H4 (see, e.g., Brightbill et al. J. Clin. Invest. 120(6):2218-2229, 2010).

Any of the preceding methods or uses may include administering a mast cell or basophil depleting agent (e.g., a mast cell or basophil depleting antibody) to the patient. In some embodiments, the antibody depletes mast cells. In other embodiments, the antibody depletes basophils. In yet other embodiments, the antibody depletes mast cells and basophils.

Any of the preceding methods or uses may include administering a PAR2 antagonist to the patient. Exemplary PAR2 antagonists include small molecule inhibitors (e.g., K-12940, K-14585, the peptide FSLLRY-NH2 (SEQ ID NO: 30), GB88, AZ3451, and AZ8838), soluble receptors, siRNAs, and anti-PAR2 antibodies (e.g., MAB3949 and Fab3949).

Any of the preceding methods or uses may include administering an IgE antagonist to the patient. In some embodiments, the IgE antagonist is an anti-IgE antibody. Any suitable anti-IgE antibody can be used. For example, the anti-IgE antibody may be any anti-IgE antibody described in U.S. Pat. No. 8,961,964, which is incorporated herein by reference in its entirety. Exemplary anti-IgE antibodies include omalizumab (XOLAIR®), E26, E27, CGP-5101 (Hu-901), HA, ligelizumab, and talizumab. In particular embodiments, the anti-IgE antibody is omalizumab (XOLAIR®).

The amino acid sequence of the heavy chain variable (VH) domain of omalizumab (XOLAIR®) is as follows (the HVR-H1, -H2, and -H3 amino acid sequences are underlined):

(SEQ ID NO: 38)
EVQLVESGGGLVQPGGSLRLSCAVSGYSITSGYSWNWIRQAPGKGLEWVA
SITYDGSTNYNPSVKGRITISRDDSKNTFYLQMNSLRAEDTAVYYCARGS
HYFGHWHFAVWGQGTLVTVSS.

The amino acid sequence of the light chain variable (VL) domain of omalizumab (XOLAIR®) is as follows (the HVR-L1, -L2, and -L3 amino acid sequences are underlined):

(SEQ ID NO: 40)
DIQLTQSPSSLSASVGDRVTITCRASQSVDYDGDSYMNWYQQKPGKAPKL
LIYAASYLESGVPSRFSGSG SGTDFTLTISSLQPEDFATYYCQQSHEDP
YTFGQGTKVEIK.

Accordingly, in some embodiments, the anti-IgE antibody includes one, two, three, four, five, or all six of the following six HVRs: (a) an HVR-H1 comprising the amino acid sequence of GYSWN (SEQ ID NO: 40); (b) an HVR-H2 comprising the amino acid sequence of SITYDGSTNYNPSVKG (SEQ ID NO: 41); (c) an HVR-H3 comprising the amino acid sequence of GSHYFGHWHFAV (SEQ ID NO: 42); (d) an HVR-L1 comprising the amino acid sequence of RASQSVDYDGDSYMN (SEQ ID NO: 43); (e) an HVR-L2 comprising the amino acid sequence of AASYLES (SEQ ID NO: 44); and (f) an HVR-L3 comprising the amino acid sequence of QQSHEDPYT (SEQ ID NO: 45). In some embodiments, the anti-IgE antibody includes (a) a VH domain comprising an amino acid sequence having at least 90%, at least 95%, or at least 99% sequence identity to the amino acid sequence of SEQ ID NO: 38; (b) a VL domain comprising an amino acid sequence having at least 90%, at least 95%, or at least 99% identity to the amino acid sequence of SEQ ID NO: 39; or (c) a VH domain as in (a) and a VL domain as in (b). In some embodiments, the VH domain comprises the amino acid sequence of SEQ ID NO: 38. In some embodiments, the VL domain comprises the amino acid sequence of SEQ ID NO: 39. In some embodiments, the VH domain comprises the amino acid sequence of SEQ ID NO: 38 and the VL domain comprises the amino acid sequence of SEQ ID NO: 39. Any of the anti-IgE antibodies described herein may be used in combination with any anti-tryptase antibody described herein, e.g., in Section VII below.

Any of the preceding methods or uses may include administering a T_{H}2 pathway inhibitor to the patient. In some embodiments, the T_{H}2 pathway inhibitor inhibits any of the targets selected from interleukin-2-inducible T cell kinase (ITK), Bruton's tyrosine kinase (BTK), Janus kinase 1 (JAK1) (e.g., ruxolitinib, tofacitinib, oclacitinib, baricitinib, filgotinib, gandotinib, lestaurtinib, momelotinib, pacrinitib, upadacitinib, peficitinib, and fedratinib), GATA binding protein 3 (GATA3), IL-9 (e.g., MEDI-528), IL-5 (e.g., mepolizumab, CAS No. 196078-29-2; resilizumab), IL-13 (e.g., IMA-026, IMA-638 (also referred to as anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody)), IL-4 (e.g., AER-001, IL-4/IL-13 trap), OX40L, TSLP, IL-25, IL-33, and IgE (e.g., XOLAIR®, QGE-031; and MEDI-4212); and receptors such as: IL-9 receptor, IL-5 receptor (e.g., MEDI-563 (benralizumab, CAS No. 1044511-01-4)), IL-4 receptor alpha (e.g., AMG-317, AIR-645), IL-13 receptoralpha1 (e.g., R-1671) and IL-13 receptoralpha2, OX40, TSLP-R, IL-7Ralpha (a co-receptor for TSLP), IL-17RB (receptor for IL-25), ST2 (receptor for IL-33), CCR3, CCR4, CRTH2 (e.g., AMG-853, AP768, AP-761, MLN6095, ACT129968), FcεRI, FcεRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL-5, IL-3, and GM-CSF (e.g., TPI ASM8).

Any of the preceding methods or uses may include administering an additional therapeutic agent to the patient. In some embodiments, the additional therapeutic agent is selected from the group consisting of a T_{H}2 pathway inhibitor, a corticosteroid, an IL-33 axis binding antagonist, a TRPA1 antagonist, a bronchodilator or asthma symptom control medication, an immunomodulator, a tyrosine kinase inhibitor, and a phosphodiesterase inhibitor. Such combination therapies are described further below.

In some embodiments, an additional therapeutic agent is an asthma therapy, as described below. Moderate asthma is currently treated with a daily inhaled anti-inflammatory-corticosteroid or mast cell inhibitor such as cromolyn sodium or nedocromil plus an inhaled beta2-agonist as needed (3-4 times per day) to relieve breakthrough symptoms or allergen- or exercise-induced asthma. Exemplary inhaled corticosteroids include QVAR®, PULMICORT®, SYMBICORT®, AEROBID®, FLOVENT®, FLONASE®, ADVAIR®, and AZMACORT®. Additional asthma therapies include long acting bronchial dilators (LABD). In certain embodiments, the LABD is a long-acting beta-2 agonist (LABA), leukotriene receptor antagonist (LTRA), long-acting muscarinic antagonist (LAMA), theophylline, or oral corticosteroids (OCS). Exemplary LABDs include SYMBICORT®, ADVAIR®, BROVANA®, FORADIL®, PERFOROMIST™, and SEREVENT®.

In some embodiments, any of the preceding methods or uses further comprises administering a bronchodilator or asthma symptom controller medication. In some embodiments, the bronchodilator or asthma controller medication is a β2-adrenergic agonist, such as a short-acting β2-agonist (SABA) (such as albuterol), or a long-acting β2-adrenergic agonist (LABA). In some embodiments, the LABA is salmeterol, abediterol, indacaterol, vilanterol, and/or formoterol (formoterol fumarate dehydrate). In some embodiments, the asthma controller medication is a Leukotriene Receptor Antagonist (LTRA). In some embodiments, the LTRA is montelukast, zafirlukast, and/or zileuton. In some embodiments, the bronchodilator or asthma controller medication is a muscarinic antagonist, such as a long-acting muscarinic acetylcholine receptor (cholinergic) antagonist (LAMA). In some embodiments, the LAMA is glycopyrronium. In some embodiments, the bronchodilator or asthma controller medication is an agonist of an ion channel such as a bitter taste receptor (such as TAS2R).

In some embodiments, any of the preceding methods or uses further comprises administering a bronchodilator. In some embodiments, the bronchodilator is an inhaled bronchodilator. In some embodiments, the inhaled bronchodilator is a β2-adrenergic agonist. In some embodiments, the β2-adrenergic agonist is a short-acting β2-adrenergic agonist (SABA). In some embodiments, the SABA is bitolterol, fenoterol, isoproterenol, levalbuterol, metaproterenol, pirbuterol, procaterol, ritodrine, albuterol, and/or terbutaline. In some embodiments, the β2-adrenergic agonist is a long-acting β2-adrenergic agonist (LABA). In some embodiments, the LABA is arformoterol, bambuterol, clenbuterol, formoterol, salmeterol, abediterol, carmoterol, indacaterol, olodaterol, and/or vilanterol. In some embodiments, the inhaled bronchodilator is a muscarinic receptor antagonist. In some embodiments, the muscarinic receptor antagonist is a short-acting muscarinic receptor antagonist (SAMA). In some embodiments, the SAMA is ipratropium bromide. In some embodiments, the muscarinic receptor antagonist is a long-acting muscarinic receptor antagonist (LAMA). In some embodiments, the LAMA is tiotropium bromide, glycopyrronium bromide, umeclidinium bromide, aclidinium bromide, and/or revefenacin. In some embodiments, the inhaled bronchodilator is a SABA/SAMA combination. In some embodiments, SABA/SAMA combination is albuterol/ipratropium. In some embodiments, the inhaled bronchodilator is a LABA/LAMA combination. In some embodiments, the LABA/LAMA combination is formoterol/aclidinium, formoterol/glycopyrronium, formoterol/tiotropium, indacaterol/glycopyrronium, indacaterol/tiotropium, olodaterol/tiotropium, salmeterol/tiotropium, and/or vilanterol/umeclidinium. In some embodiments, the inhaled bronchodilator is a bifunctional bronchodilator. In some embodiments, the bifunctional bronchodilator is a muscarinic antagonist/β2-agonist (MABA). In some embodiments, the MABA is batefenterol, THRX 200495, AZD 2115, LAS 190792, TE13252, PF-3429281 and/or PF-4348235. In some embodiments, the inhaled bronchodilator is an agonist of TAS2R. In some embodiments, the bronchodilator is a nebulized SABA. In some embodiments, the nebulized SABA is albuterol and/or levalbuterol. In some embodiments, the bronchodilator is a nebulized LABA. In some embodiments, the nebulized LABA is arformoterol and/or formoterol. In some embodiments, the bronchodilator is a nebulized SAMA. In some embodiments, the nebulized SAMA is ipratropium. In some embodiments, the bronchodilator is a nebulized LAMA. In some embodiments, the nebulized LAMA is glycopyrronium and/or revefenacin. In some embodiments, the bronchodilator is a nebulized SABA/SAMA combination. In some embodiments, the nebulized SABA/SAMA combination is albuterol/ipratropium. In some embodiments, the bronchodilator is a leukotriene receptor antagonist (LTRA). In some embodiments, the LTRA is montelukast, zafirlukast, and/or zileuton. In some embodiments, the bronchodilator is a methylxanthine. In some embodiments, the methylxanthine is theophylline.

In some embodiments, any of the preceding methods or uses further comprises administering an immunomodulator. In some embodiments, the method further comprises administering cromolyn. In some embodiments, the method further comprises administering methylxanthine. In some embodiments, the methylxanthine is theophylline or caffeine.

In some embodiments, any of the preceding methods or uses further comprises administering one or more corticosteroids, such as an inhaled corticosteroid (ICS) or an oral corticosteroid. Non-limiting exemplary corticosteroids include inhaled corticosteroids, such as beclomethasone dipropionate, budesonide, ciclesonide, flunisolide, fluticasone propionate, fluticasone furoate, mometasone, and/or triamcinolone acetonide and oral corticosteroids, such as methylprednisolone, prednisolone, and prednisone. In some embodiments, the corticosteroid is an ICS. In some embodiments, the ICS is beclomethasone, budesonide, flunisolide, fluticasone furoate, fluticasone propionate, mometasone, ciclesonide, and/or triamcinolone. In some embodiments, the method further comprises administering an ICS/LABA and/or LAMA combination. In some embodiments, the ICS/LABA and/or LAMA combination is fluticasone propionate/salmeterol, budesonide/formoterol, mometasone/formoterol, fluticasone furoate/vilanterol, fluticasone propionate/formoterol, beclomethasone/formoterol, fluticasone furoate/umeclidinium, fluticasone furoate/vilanterol/umeclidinium, fluticasone/salmeterol/tiotropium, beclomethasone/formoterol/glycopyrronium, budesonide/formoterol/glycopyrronium, and/or budesonide/formoterol/tiotropium. In some embodiments, the method further comprises administering a nebulized corticosteroid. In some embodiments, the nebulized corticosteroid is budesonide. In some embodiments, the method further comprises administering an oral or intravenous corticosteroid. In some embodiments, the oral or intravenous corticosteroid is prednisone, prednisolone, methylprednisolone, and/or hydrocortisone.

In some embodiments, any of the preceding methods or uses further comprises administering one or more active ingredients selected from an aminosalicylate; a steroid; a biological; a thiopurine; methotrexate; a calcineurin inhibitor, e.g., cyclosporine or tacrolimus; and an antibiotic. In some embodiments, the method comprises administering the further active ingredient in an oral or topical formulation. Examples of aminosalicylates include 4-aminosalicylic acid, sulfasalazine, balsalazide, olsalazine and mesalazine, in forms like Eudragit-S-coated, pH-dependent mesalamine, ethylcellulose-coated mesalamine, and multimatrix-release mesalamine. Examples of a steroid include corticosteroids or glucocorticosteroids. Examples of a corticosteroid include prednisone and hydrocortisone or methylprednisolone, or a second generation corticosteroid, e.g., budesonide or azathioprine; e.g., in forms like a hydrocortisone enema or a hydrocortisone foam, Examples of biologicals include etanercept; an antibody to tumor necrosis factor alpha, e.g., infliximab, adalimumab or certolizumab; an antibody to IL-12 and IL-23, e.g., ustekinumab; vedolizumab; etrolizumab, and natalizumab. Examples of thiopurines include azathioprine, 6-mercaptopurine and thioguanine. Examples of antibiotics include vancomycin, rifaximin, metronidazole, trirnethoprim, sulfamethoxazole, diaminodiphenyl sulfone, and ciprofloxacin; and antiviral agents like ganciclovir.

In some embodiments, any of the preceding methods or uses further comprises administering an antifibrotic agent. In some embodiments, the antifibrotic agent inhibits transforming growth factor beta (TGF-β)-stimulated collagen synthesis, decreases the extracellular matrix, and/or blocks fibroblast proliferation. In some embodiments, the antifibrotic agent is pirfenidone. In some embodiments, the antifibrotic agent is PBI-4050. In some embodiments, the antifibrotic agent is tipelukast.

In some embodiments, any of the preceding methods or uses further comprises administering a tyrosine kinase inhibitor. In some embodiments, the tyrosine kinase inhibitor inhibits a tyrosine kinase that mediates elaboration of one or more fibrogenic growth factors. In some embodiments, the fibrogenic growth factor is platelet-derived growth factor, vascular endothelial growth factor, and/or fibroblast growth factor. In some embodiments, the tyrosine kinase inhibitor is imatinib and/or nintedanib. In some embodiments, the tyrosine kinase inhibitor is nintedanib. In some embodiments, the method further comprises administering an antidiarrheal agent. In some embodiments, the antidiarrheal agent is loperamide.

In some embodiments, any of the preceding methods or uses further comprises administering an antibody. In some embodiments, the antibody is an anti-interleukin (IL)-13 antibody. In some embodiments, the anti-IL-13 antibody is tralokinumab. In some embodiments, the antibody is an anti-IL-4/anti-IL-13 antibody. In some embodiments, the anti-IL-4/anti-IL-13 antibody is SAR 156597. In some embodiments, the antibody is an anti-connective tissue growth factor (CTGF) antibody. In some embodiments, the anti-CTGF antibody is FG-3019. In some embodiments, the antibody is an anti-lysyl oxidase-like 2 (LOXL2) antibody. In some embodiments, the anti-LOXL2 antibody is simtuzumab. In some embodiments, the antibody is an anti-αvβ6 integrin receptor antibody. In some embodiments, the anti-αvβ6 integrin receptor antibody is STX-100. In some embodiments, the antibody is a monoclonal antibody.

In some embodiments, any of the preceding methods or uses further comprises administering a lysophosphatidic acid-1 (LPA1) receptor antagonist. In some embodiments, the LPA1 receptor antagonist is BMS-986020. In some embodiments, the method further comprises administering a galectin 3 inhibitor. In some embodiments, the galectin 3 inhibitor is TD-139.

In some embodiments, any of the preceding methods or uses further comprises administering a palliative therapy. In some embodiments, the palliative therapy comprises one or more of an antibiotic, an anxiolytic, a corticosteroid, and an opioid. In some embodiments, the antibiotic is a broad-spectrum antibiotic. In some embodiments, the antibiotic is penicillin, a β-lactamase inhibitor, and/or a cephalosporin. In some embodiments, the antibiotic is piperacillin/tazobactam, cefixime, ceftriaxone and/or cefdinir. In some embodiments, the anxiolytic is alprazolam, buspirone, chlorpromazine, diazepam, midazolam, lorazepam, and/or promethazine. In some embodiments, the corticosteroid is a glucocorticosteroid. In some embodiments, the glucocorticosteroid is prednisone, prednisolone, methylprednisolone, and/or hydrocortisone. In some embodiments, the opioid is morphine, codeine, dihydrocodeine, and/or diamorphine.

In some embodiments, any of the preceding methods or uses further comprises administering an antibiotic. In some embodiments, the antibiotic is a macrolide. In some embodiments, the macrolide is azithromycin, and/or clarithromycin. In some embodiments, the antibiotic is doxycycline. In some embodiments, the antibiotic is trimethoprim/sulfamethoxazole. In some embodiments, the antibiotic is a cephalosporin. In some embodiments, the cephalosporin is cefepime, cefixime, cefpodoxime, cefprozil, ceftazidime, and/or cefuroxime. In some embodiments, the antibiotic is penicillin. In some embodiments, the antibiotic is amoxicillin, ampicillin, and/or pivampicillin. In some embodiments, the antibiotic is a penicillin/β-lactamase inhibitor combination. In some embodiments, the penicillin/β-lactamase inhibitor combination is amoxicillin/clavulanate and/or piperacillin/tazobactam. In some embodiments, the antibiotic is a fluoroquinolone. In some embodiments, the fluoroquinolone is ciprofloxacin, gemifloxacin, levofloxacin, moxifloxacin, and/or ofloxacin.

In some embodiments, any of the preceding methods or uses further comprises administering a phosphodiesterase inhibitor. In some embodiments, the phosphodiesterase inhibitor is a phosphodiesterase type 5 inhibitor. In some embodiments, the phosphodiesterase inhibitor is avanafil, benzamidenafil, dasantafil, icariin, lodenafil, mirodenafil, sildenafil, tadalafil, udenafil, and/or vardenafil. In some embodiments, the PDE inhibitor is a PDE-4 inhibitor. In some embodiments, the PDE-4 inhibitor is roflumilast, cilomilast, tetomilast, and/or CHF6001. In some embodiments, the PDE inhibitor is a PDE-3/PDE-4 inhibitor. In some embodiments, the PDE-3/PDE-4 inhibitor is RPL-554.

In some embodiments, any of the preceding methods or uses further comprises administering a cytotoxic and/or immunosuppressive agent. In some embodiments, the cytotoxic and/or immunosuppressive agent is azathioprine, colchicine, cyclophosphamide, cyclosporine, methotrexate, penicillamine, and/or thalidomide. In some embodiments, the method further comprises administering an agent that restores depleted glutathione levels in the lung. In some embodiments, the agent that restores depleted glutathione levels in the lung is N-acetylcysteine. In some embodiments, the method further comprises administering an anticoagulant. In some embodiments, the anticoagulant is warfarin, heparin, activated protein C, and/or tissue factor pathway inhibitor.

In some embodiments, any of the preceding methods or uses further comprises administering an endothelin receptor antagonist. In some embodiments, the endothelin receptor antagonist is bosentan, macitentan and/or ambrisentan. In some embodiments, the method further comprises administering a TNF-α antagonist. In some embodiments, the TNF-α antagonist comprises one or more of etanercept, adalimumab, infliximab, certolizumab, and golimumab. In some embodiments, the method further comprises administering interferon gamma-1b.

In some embodiments, any of the preceding methods or uses further comprises administering an interleukin (IL) inhibitor. In some embodiments, the IL inhibitor is an IL-5 inhibitor. In some embodiments, the IL-5 inhibitor is mepolizumab and/or benralizumab. In some embodiments, the IL inhibitor is an IL-17A inhibitor. In some embodiments, the IL-17A inhibitor is CNTO-6785.

In some embodiments, any of the preceding methods or uses further comprises administering a p38 mitogen-activated protein kinase (MAPK) inhibitor. In some embodiments, the p38 MAPK inhibitor is losmapimod and/or AZD-7624. In some embodiments, the method further comprises administering a CXCR2 antagonist. In some embodiments, the CXCR2 antagonist is danirixin.

In some embodiments, any of the preceding methods or uses further comprises vaccination. In some embodiments, the vaccination is vaccination against pneumococci and/or influenza. In some embodiments, the vaccination is vaccination against Streptococcus pneumoniae and/or influenza. In some embodiments, the method further comprises administering an antiviral therapy. In some embodiments, the antiviral therapy is oseltamivir, peramivir, and/or zanamivir.

In some embodiments, any of the preceding methods or uses further comprises prevention of gastroesophageal reflux and/or recurrent microaspiration.

In some embodiments, any of the preceding methods or uses further comprises ventilatory support. In some embodiments, the ventilatory support is mechanical ventilation. In some embodiments, the ventilatory support is noninvasive ventilation. In some embodiments, the ventilatory support is supplemental oxygen. In some embodiments, the method further comprises pulmonary rehabilitation.

In some embodiments, any of the preceding methods or uses further comprises lung transplantation. In some embodiments, the lung transplantation is single lung transplantation. In some embodiments, the lung transplantation is bilateral lung transplantation.

In some embodiments, any of the preceding methods or uses further comprises a non-pharmacological intervention. In some embodiments, the non-pharmacological intervention is smoking cessation, a healthy diet, and/or regular exercise. In some embodiments, the method further comprises administering a pharmacological aid for smoking cessation. In some embodiments, the pharmacological aid for smoking cessation is nicotine replacement therapy, bupropion, and/or varenicline. In some embodiments, the non-pharmacological intervention is lung therapy. In some embodiments, the lung therapy is pulmonary rehabilitation and/or supplemental oxygen. In some embodiments, the non-pharmacological intervention is lung surgery. In some embodiments, the lung surgery is lung volume reduction surgery, single lung transplantation, bilateral lung transplantation, or bullectomy. In some embodiments, the non-pharmacological intervention is the use of a device. In some embodiments, the device is a lung volume reduction coil, an exhale airway stent, and/or a nasal ventilatory support system.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. The combined administration includes co-administration, using separate formulations or a single pharmaceutical formulation, and consecutive administration in either order, wherein preferably there is a time period while both (or all) active agents simultaneously exert their biological activities. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. When administered sequentially, the combination may be administered in two or more administrations.

Such combination therapies noted above encompass combined administration (where two or more therapeutic agents are included in the same or separate formulations), and separate administration, in which case, administration of an agent (e.g., a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist)), or a pharmaceutical composition thereof, can occur prior to, simultaneously, and/or following, administration of the additional therapeutic agent(s). In one embodiment, administration of agent (e.g., a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist)), or a pharmaceutical composition thereof, and administration of an additional therapeutic agent occur within about one month; or within about one, two, or three weeks; or within about one, two, three, four, five, or six days; or within about 1, 2, 3, 4, 5, 6, 7, 8, or 9 hours; or within about 1, 5, 10, 20, 30, 40, or 50 minutes, of each other. For embodiments involving sequential administration, the agent (e.g., a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, or a combination thereof (e.g., a tryptase antagonist and an IgE antagonist)) may be administered prior to or after administration of the additional therapeutic agent(s).

In any of the preceding methods or uses, the therapy (e.g., a therapy including a tryptase antagonist, an FcεR antagonist, an IgE⁺ B cell depleting antibody, a mast cell or basophil depleting antibody, a PAR2 antagonist, an IgE antagonist, or a combination thereof (e.g., a tryptase antagonist and an IgE antagonist)), and any additional therapeutic agent, can be administered by any suitable means, including parenterally, intraperitoneally, intramuscularly, intravenously, intradermally, percutaneously, intraarterially, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, intravitreally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The administration may be systemic or local. In addition, the antagonist may suitably be administered by pulse infusion, e.g., with declining doses of the antagonist.

Any therapeutic agent, e.g., a tryptase antagonist, an FcεR antagonist, an IgE⁺ B cell depleting antibody, a mast cell or basophil depleting antibody, a PAR2 antagonist, an IgE antagonist, a combination thereof (e.g., a tryptase antagonist and an IgE antagonist), any additional therapeutic agent, or pharmaceutical compositions thereof, would be formulated, dosed, and administered in a fashion consistent with good medical practice. Such dosages are known in the art. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The tryptase antagonist, FcεR antagonist, IgE⁺ B cell depleting antibody, mast cell or basophil depleting antibody, a PAR2 antagonist, IgE antagonist, or pharmaceutical composition thereof, need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

As one example, for the prevention or treatment of disease, the appropriate dosage of an antibody (e.g., an anti-tryptase antibody, an anti-IgE antibody (e.g., XOLAIR®), an IgE+ B cell depleting antibody (e.g., an anti-M1′ domain antibody (e.g., quilizumab)), a mast cell or basophil depleting antibody, or an anti-PAR2 antibody) (when used alone or in combination with one or more other additional therapeutic agents) will depend on the type of disease to be treated, the type of antibody, the severity and course of the disease, whether the antibody is administered for preventive or therapeutic purposes, previous therapy, the patients clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg to 10 mg/kg) of antibody can be an initial candidate dosage for administration to the patient, whether, for example, by one or more separate administrations, or by continuous infusion. One typical daily dosage might range from about 1 μg/kg to 200 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week, every two weeks, every three weeks, or every four weeks (e.g., such that the patient receives from about two to about twenty, or e.g., about six doses of the antibody). For example, a dose may be administered once per month. An initial higher loading dose, followed by one or more lower doses may be administered. However, other dosage regimens may be useful. The progress of this therapy is easily monitored by conventional techniques and assays. In some instances, a dose of about 50 mg/mL to about 200 mg/mL (e.g., about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL of an antibody may be administered. In some embodiments, XOLAIR® (omalizumab) dosing for asthma patients can be determined based on body weight and pretreatment IgE levels using approaches known in the art. XOLAIR® (omalizumab) can be administered by subcutaneous injection every four weeks at 300 mg or 150 mg per dose for treatment of CIU.

In any of the preceding methods or uses, in some embodiments, the mast cell-mediated inflammatory disease is selected from the group consisting of asthma, atopic dermatitis, urticaria (e.g., CSU or CIU), systemic anaphylaxis, mastocytosis, chronic obstructive pulmonary disease (COPD), idiopathic pulmonary fibrosis (IPF), and eosinophilic esophagitis.

In some embodiments of any of the preceding methods or uses, the mast cell-mediated inflammatory disease is asthma. In some embodiments, the asthma is persistent chronic severe asthma with acute events of worsening symptoms (exacerbations or flares) that can be life threatening. In some embodiments, the asthma is atopic (also known as allergic) asthma, non-allergic asthma (e.g., often triggered by infection with a respiratory virus (e.g., influenza, parainfluenza, rhinovirus, human metapneurnovirus, and respiratory syncytial virus) or inhaled irritant (e.g., air pollutants, smog, diesel particles, volatile chemicals and gases indoors or outdoors, or even by cold dry air).

In some embodiments of any of the preceding methods or uses, the asthma is intermittent or exercise-induced, asthma due to acute or chronic primary or second-hand exposure to “smoke” (typically cigarettes, cigars, or pipes), inhaling or “vaping” (tobacco, marijuana, or other such substances), or asthma triggered by recent ingestion of aspirin or related NSAIDS. In some embodiments, the asthma is mild, or corticosteroid naïve asthma, newly diagnosed and untreated asthma, or not previously requiring chronic use of inhaled topical or systemic steroids to control the symptoms (cough, wheeze, shortness of breath/breathlessness, or chest pain). In some embodiments, the asthma is chronic, corticosteroid resistant asthma, corticosteroid refractory asthma, or asthma uncontrolled on corticosteroids or other chronic asthma controller medications.

In some embodiments of any of the preceding methods or uses, the asthma is moderate to severe asthma. In certain embodiments, the asthma is T_{H}2-high asthma. In some embodiments, the asthma is severe asthma. In some embodiments, the asthma is atopic asthma, allergic asthma, non-allergic asthma (e.g., due to infection and/or respiratory syncytial virus (RSV)), exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, or asthma uncontrolled on corticosteroids. In some embodiments, the asthma is eosinophilic asthma. In some embodiments, the asthma is allergic asthma. In some embodiments, the individual has been determined to be Eosinophilic Inflammation Positive (EIP). See WO2015/061441. In some embodiments, the asthma is periostin-high asthma (e.g., having periostin level at least about any of 20 ng/ml, 25 ng/ml, or 50 ng/ml serum). In some embodiments, the asthma is eosinophil-high asthma (e.g., at least about any of 150, 200, 250, 300, 350, 400 eosinophil counts/ml blood). In certain embodiments, the asthma is T_{H}2-low asthma. In some embodiments, the individual has been determined to be Eosinophilic Inflammation Negative (EIN). See WO2015/061441. In some embodiments, the asthma is periostin-low asthma (e.g., having periostin level less than about 20 ng/ml serum). In some embodiments, the asthma is eosinophil-low asthma (e.g., less than about 150 eosinophil counts/μl blood or less than about 100 eosinophil counts/μl blood).

For example, in particular embodiments of any of the preceding methods or uses, the asthma is moderate to severe asthma. In some embodiments, the asthma is uncontrolled on a corticosteroid. In some embodiments, the asthma is T_{H}2 high asthma or T_{H}2 low asthma. In particular embodiments, the asthma is T_{H}2 high asthma.

### III. Diagnostic Methods of the Invention

The present invention features methods of determining whether patients having a mast cell-mediated inflammatory disease (e.g., asthma) are likely to respond to a therapy (e.g., a therapy comprising an agent selected from the group consisting of a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, and a combination thereof (e.g., a tryptase antagonist and an IgE antagonist)), methods of selecting a therapy for a patient having a mast cell-mediated inflammatory disease, methods for assessing a response of a patient having mast cell-mediated inflammatory disease, and methods for monitoring the response of a patient having a mast cell-mediated inflammatory disease. In some embodiments, the therapy is a mast-cell directed therapy (e.g. a therapy that includes a tryptase antagonist, an IgE antagonist, an IgE⁺ B cell depleting antibody, a mast cell or basophil depleting antibody, and/or a PAR2 antagonist). In some embodiments, the therapy includes a tryptase antagonist (e.g., an anti-tryptase antibody, e.g., any anti-tryptase antibody described herein or in WO 2018/148585) and an IgE antagonist (e.g., an anti-IgE antibody, e.g., omalizumab (XOLAIR®)).

The presence and/or expression level of the biomarker of the invention (e.g., an active tryptase allele count and/or tryptase) can be determined using any of the assays described herein or by any method or assay known in the art. In some embodiments, the methods further involve administering a therapy to the patient, for example, as described in Section II of the Detailed Description of the Invention above. The methods may be conducted in a variety of assay formats, including assays detecting genetic information (e.g., DNA or RNA sequencing), genetic or protein expression (such as polymerase chain reaction (PCR) and enzyme immunoassays), and biochemical assays detecting appropriate activity, for example, as described below.

For example, in one aspect, the invention features a method of determining whether a patient having a mast cell-mediated inflammatory disease is likely to respond to a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)), the method including: (a) determining in a sample from a patient having a mast cell-mediated inflammatory disease the patient's active tryptase allele count; and (b) identifying the patient as likely to respond to a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) based on the patient's active tryptase allele count, wherein an active tryptase allele count at or above a reference active tryptase allele count indicates that the patient has an increased likelihood of being responsive to the therapy. In some embodiments, the method further includes administering the therapy to the patient.

In another example, the invention features a method of determining whether a patient having a mast cell-mediated inflammatory disease is likely to respond to a mast cell-directed therapy (e.g., a therapy comprising an agent selected from the group consisting of a tryptase antagonist, an IgE antagonist, an IgE⁺ B cell depleting antibody, a mast cell or basophil depleting antibody, a protease activated receptor 2 (PAR2) antagonist, and a combination thereof (e.g., a tryptase antagonist and an IgE antagonist)), the method including: (a) determining the expression level of tryptase in a sample from a patient having a mast cell-mediated inflammatory disease; and (b) identifying the patient as likely to respond to a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) based on the expression level of tryptase in the sample from the patent, wherein an expression level of tryptase in the sample at or above a reference level of tryptase indicates that the patient has an increased likelihood of being responsive to the therapy. In some embodiments, the method further includes administering the therapy to the patient.

In some embodiments of any of the preceding methods, the patient has been identified as having a level of a Type 2 biomarker in a sample from the patient that is below a reference level of the Type 2 biomarker. In some embodiments, the agent is administered to the patient as a monotherapy.

In some embodiments of any of the preceding methods, the patient has been identified as having a level of a Type 2 biomarker in a sample from the patient that is at or above a reference level of the Type 2 biomarker. In some embodiments, the method further comprises administering a T_{H}2 pathway inhibitor to the patient.

In another aspect, the invention features a method of determining whether a patient having a mast cell-mediated inflammatory disease is likely to respond to a therapy comprising an IgE antagonist or an FcεR antagonist that includes (a) determining in a sample from a patient having a mast cell-mediated inflammatory disease the patient's active tryptase allele count; and (b) identifying the patient as likely to respond to a therapy comprising an IgE antagonist or an FcεR antagonist based on the patient's active tryptase allele count, wherein an active tryptase allele count below a reference active tryptase allele count indicates that the patient has an increased likelihood of being responsive to the therapy. In some embodiments, the method further includes administering the therapy to the patient.

In another example, the invention features a method of determining whether a patient having a mast cell-mediated inflammatory disease is likely to respond to a therapy comprising an IgE antagonist or an FcεR antagonist that includes (a) determining the expression level of tryptase in a sample from a patient having a mast cell-mediated inflammatory disease; and (b) identifying the patient as likely to respond to a therapy comprising an IgE antagonist or an FcεR antagonist based on the expression level of tryptase in the sample from the patient, wherein an expression level of tryptase in the sample from the patient below a reference level of tryptase indicates that the patient has an increased likelihood of being responsive to the therapy. In some embodiments, the method further includes administering the therapy to the patient.

In some embodiments of any of the preceding methods, the patient has been identified as having a level of a Type 2 biomarker in a sample from the patient that is at or above a reference level of the Type 2 biomarker. In some embodiments, the method further comprises administering an additional T_{H}2 pathway inhibitor to the patient.

In a further example, the invention features a method of selecting a therapy for a patient having a mast cell-mediated inflammatory disease that includes (a) determining in a sample from a patient having a mast cell-mediated inflammatory disease the patient's active tryptase allele count; and (b) selecting for the patient: (i) a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) if the patient's active tryptase allele count is at or above a reference active tryptase allele count, or (ii) a therapy comprising an IgE antagonist or an FcεR antagonist if the patient's active tryptase allele count is below a reference active tryptase allele count. In some embodiments, the method further includes administering the therapy selected in accordance with (b) to the patient.

In yet another example, the invention features a method of selecting a therapy for a patient having a mast cell-mediated inflammatory disease that includes (a) determining the expression level of tryptase in a sample from a patient having a mast cell-mediated inflammatory disease; and (b) selecting for the patient:

(i) a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) if the expression level of tryptase in the sample from the patient is at or above a reference level of tryptase, or (ii) a therapy comprising an IgE antagonist or an FcεR antagonist if the expression level of tryptase in the sample from the patient is below a reference level of tryptase. In some embodiments, the method further includes administering the therapy selected in accordance with (b) to the patient.

In some embodiments of any of the preceding aspects, the patient has been identified as having a level of a Type 2 biomarker in a sample from the patient that is below a reference level of the Type 2 biomarker. In some embodiments, the agent is administered to the patient as a monotherapy.

In some embodiments of any of the preceding aspects, the patient has been identified as having a level of a Type 2 biomarker in a sample from the patient that is at or above a reference level of the Type 2 biomarker, and the method further comprises selecting a combination therapy that comprises a T_{H}2 pathway inhibitor. In some embodiments, the method further comprises administering a T_{H}2 pathway inhibitor (or an additional T_{H}2 pathway inhibitor) to the patient.

The invention also features a method for assessing a response of a patient having a mast cell-mediated inflammatory disease to treatment with a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)), the method including: (a) determining the expression level of tryptase in a sample from a patient having a mast cell-mediated inflammatory disease at a time point during or after administration of a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)) to the patient; and (b) maintaining, adjusting, or stopping the treatment based on a comparison of the expression level of tryptase in the sample with a reference level of tryptase, wherein a change in the expression level of tryptase in the sample from the patient compared to the reference level is indicative of a response to treatment with the therapy. In some embodiments, the change is an increase in the expression level of tryptase and the treatment is maintained. In other embodiments, the change is a decrease in the expression level of tryptase and the treatment is adjusted or stopped.

In another example, the invention features a method for monitoring the response of a patient having a mast cell-mediated inflammatory disease treated with a mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist)), the method including: (a) determining the expression level of tryptase in a sample from the patient at a time point during or after administration of the mast cell-directed therapy (e.g., a therapy comprising an agent selected from the group consisting of a tryptase antagonist, an IgE antagonist, an IgE⁺ B cell depleting antibody, a mast cell or basophil depleting antibody, a PAR2 antagonist, and a combination thereof (e.g., a tryptase antagonist and an IgE antagonist) to the patient); and (b) comparing the expression level of tryptase in the sample from the patient with a reference level of tryptase, thereby monitoring the response of the patient undergoing treatment with the therapy. In some embodiments, the change is an increase in the expression level of tryptase and the treatment is maintained. In other embodiments, the change is a decrease in the expression level of tryptase and the treatment is adjusted or stopped.

In some embodiments of any of the preceding methods, the active tryptase allele count has been determined by sequencing the TPSAB1 and TPSB2 loci of the patient's genome. Any suitable sequencing approach can be used, for example, Sanger sequencing or massively parallel (e.g., ILLUMINA®) sequencing. In some embodiments, the TPSAB1 locus is sequenced by a method comprising (i) amplifying a nucleic acid from the subject in the presence of a first forward primer comprising the nucleotide sequence of 5′-CTG GTG TGC AAG GTG AAT GG-3′ (SEQ ID NO: 31) and a first reverse primer comprising the nucleotide sequence of 5″-AGG TCC AGO ACT CAG GAG GA-3′ (SEQ ID NO: 32) to form a TPSAB1 amplicon, and (ii) sequencing the TPSAB1 amplicon. In some embodiments, sequencing the TPSAB1 amplicon comprises using the first forward primer and the first reverse primer. In some embodiments, the TPSB2 locus is sequenced by a method comprising (i) amplifying a nucleic acid from the subject in the presence of a second forward primer comprising the nucleotide sequence of 5′-GCA GGT GAG COT GAG AGT CC-3′ (SEQ ID NO: 33) and a second reverse primer comprising the nucleotide sequence of 5r-GGG ACC ITC ACC TGC ITC AG-3′ (SEQ ID NO: 34) to form a TPSB2 amplicon, and (ii) sequencing the TPSB2 amplicon. In some embodiments, sequencing the TPSB2 amplicon comprises Lasing the second forward primer and a sequencing reverse primer comprising the nucleotide sequence of 5′-CAG CCA GIG ACC CAG CAC-3′ (SEQ ID NO: 35). In some embodiments, the active tryptase allele count may be determined by determining the presence of any variation in the TPSAB1 and TPSB2 loci of the patient's genome. In some embodiments, the active tryptase allele count is determined by the formula: 4—the sum of the number of tryptase alpha and tryptase beta III frame-shift (beta III^{FS}) alleles in the patient's genotype. In some embodiments, tryptase alpha is detected by detecting the c733 G>C SNP at TPSAB1. In some embodiments, detecting the c733 G>A SNP at TPSAB1 comprises detecting the patient's genotype at the polymorphism CTGCAGGCGGGCGTGGTCAGCTGGG[G/A]CGAGGGCTGTGCCCAGCCCAACCGG (SEQ ID NO: 36), wherein the presence of an A at the c733 G>A SNP indicates tryptase alpha. In some embodiments, tryptase beta III^{FS} is detected by detecting a c980_981insC mutation at TPSB2. In some embodiments, detecting a c980_981insC mutation at TPSB2 comprises detecting the nucleotide sequence CACACGGTCACCCTGCCCCCTGCCTCAGAGACCTTCCCCCCC (SEQ ID NO: 37). In some embodiments of any of the preceding methods, the patient has an active tryptase allele count of 3 or 4. In some embodiments, the active tryptase allele count is 3. In other embodiments, the active tryptase allele count is 4.

In other embodiments of any of the preceding methods, the patient has an active tryptase allele count of 0, 1, or 2. In some embodiments, the active tryptase allele count is 0. In some embodiments, the active tryptase allele count is 1. In other embodiments, the active tryptase allele count is 2.

In some embodiments of any of the preceding methods, the reference active tryptase allele count can be determined in a reference sample, a reference population, and/or be a pre-assigned value (e.g., a cut-off value which was previously determined to significantly (e.g., statistically significantly) separate a first subset of individuals from a second subset of individuals (e.g., in terms of response to a therapy (e.g., a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof (e.g., a tryptase antagonist and an IgE antagonist))). In some embodiments, the reference active tryptase allele count is a pre-determined value. In some embodiments, the reference active tryptase allele count is predetermined in the mast cell-mediated inflammatory disease to which the patient belongs (e.g., asthma). In certain embodiments, the active tryptase allele count is determined from the overall distribution of the values in a mast cell-mediated inflammatory disease (e.g., asthma) investigated or in a given population. In some embodiments, a reference active tryptase allele count is an integer in the range of from 0 to 4 (e.g., 0, 1, 2, 3, or 4). In particular embodiments, a reference active tryptase allele count is 3.

Any of the preceding methods can include determining the expression level of one or more Type 2 biomarkers. In some embodiments, the Type 2 biomarker is a T_{H}2 cell-related cytokine, periostin, eosinophil count, an eosinophil signature, FeNO, or IgE. In some embodiments, the T_{H}2 cell-related cytokine is IL-13, IL-4, IL-9, or IL-5.

In any of the preceding methods, the genotype of a patient can be determined using any of the methods or assays described herein (e.g., in Section IV of the Detailed Description of the Invention or in Example 1) or that are known in the art.

In some embodiments of any of the preceding methods, the expression level of the biomarker is a protein expression level. For example, in some embodiments, the protein expression level is measured using an immunoassay (e.g., a multiplexed immunoassay), ELISA, Western blot, or mass spectrometry. In some embodiments, the protein expression level of tryptase is an expression level of active tryptase. In other embodiments, the protein expression level of tryptase is an expression level of total tryptase.

In other embodiments of any of the preceding methods, the expression level of the biomarker is an mRNA expression level. For example, in some embodiments, the mRNA expression level is measured using a PCR method (e.g., qPCR) or a microarray chip.

In some embodiments of any of the preceding methods, the reference level of the biomarker is a level of the biomarker determined in a group of individuals having asthma. For example, in some embodiments, the reference level is a median level.

Any suitable sample derived from the patient may be used in any of the preceding methods. For example, in some embodiments, the sample derived from the patient is a blood sample (e.g., a whole blood sample, a serum sample, a plasma sample, or a combination thereof), a tissue sample, a sputum sample, a bronchiolar lavage sample, a mucosal lining fluid (MLF) sample, a bronchosorption sample, or a nasosorption sample.

In any of the preceding methods, the expression level of a biomarker of the invention (e.g., tryptase) in a sample derived from the patient may be changed at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker. For instance, in some embodiments, the expression level of a biomarker of the invention in a sample derived from the patient may be increased at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker. In other embodiments, the expression level of a biomarker of the invention in a sample derived from the patient may be decreased at least about 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, or more relative to a reference level of the biomarker.

In some embodiments of any of the preceding methods, the reference level may be set to any percentile between, for example, the 20^{th} percentile and the 99^{th} percentile (e.g., the 20^{th}, 25^{th}, 30^{th}, 35^{th}, 40^{th}, 45^{th}, 50^{th}, 55^{th}, 60^{th}, 65^{th}, 70^{th}, 75^{th}, 80^{th}, 85^{th}, 90^{th}, 95^{th}, or 99^{th} percentile) of the overall distribution of the expression level of a biomarker (e.g., tryptase), for example, in healthy subjects or in patients having a disorder (e.g., a mast cell-mediated inflammatory disease (e.g., asthma)). In some embodiments, the reference level may be set to the 25th percentile of the overall distribution of the values in a population of patients having asthma. In other embodiments, the reference level may be set to the 50th percentile of the overall distribution of the values in a population of patients having a mast cell-mediated inflammatory disease (e.g., asthma). In yet other embodiments, the reference level may be the median of the overall distribution of the values in a population of patients having a mast cell-mediated inflammatory disease (e.g., asthma).

In any of the preceding methods, the patient may have an elevated level of a T_{H}2 biomarker relative to a reference level. In some embodiments, the T_{H}2 biomarker is selected from the group consisting of serum periostin, fractional exhaled nitric oxide (FeNO), sputum eosinophil count, and peripheral blood eosinophil count. In some embodiments, the T_{H}2 biomarker is serum periostin. For example, the patient may have a serum periostin level of about 20 ng/ml or higher (e.g., about 20 ng/ml, about 25 ng/ml, about 30 ng/ml, about 35 ng/ml, about 40 ng/ml, about 45 ng/ml, about 50 ng/ml, or higher). In other embodiments, the patient may have a serum periostin level of about 50 ng/ml or higher (e.g., about 50 ng/ml, about 55 ng/ml, about 60 ng/ml, about 65 ng/ml, about 70 ng/ml, about 75 ng/ml, about 80 ng/ml, or higher). Serum periostin levels may be determined using any suitable method, for example an enzyme-linked immunosorbent assay (ELISA). Suitable approaches are described herein.

In some embodiments of any of the preceding methods, the therapy includes a tryptase antagonist. The tryptase antagonist may be a tryptase alpha antagonist (e.g., a tryptase alpha 1 antagonist) or a tryptase beta antagonist (e.g., a tryptase beta 1, tryptase beta 2, and/or tryptase beta 3 antagonist). In some embodiments, the tryptase antagonist is a tryptase alpha antagonist and a tryptase beta antagonist. In some embodiments, the tryptase antagonist (e.g., the tryptase alpha antagonist and/or the tryptase beta antagonist) is an anti-tryptase antibody (e.g., an anti-tryptase alpha antibody and/or an anti-tryptase beta antibody). Any anti-tryptase antibody described in Section VII below can be used.

In some embodiments of any of the preceding methods, the therapy includes an FcεR antagonist. In some embodiments, the FcεR antagonist inhibits FcεRIα, FcεRIβ, and/or FcεRIγ. In other embodiments, the FcεR antagonist inhibits FcεRII. In yet other embodiments, the FcεR antagonist inhibits a member of the FcεR signaling pathway. For example, in some embodiments, the FcεR antagonist inhibits tyrosine-protein kinase Lyn (Lyn), Bruton's tyrosine kinase (BTK), tyrosine-protein kinase Fyn (Fyn), spleen associated tyrosine kinase (Syk), linker for activation of T cells (LAT), growth factor receptor bound protein 2 (Grb2), son of sevenless (Sos), Ras, Raf-1, mitogen-activated protein kinase kinase 1 (MEK), mitogen-activated protein kinase 1 (ERK), cytosolic phospholipase A2 (cPLA2), arachidonate 5-lipoxygenase (5-LO), arachidonate 5-lipoxygenase activating protein (FLAP), guanine nucleotide exchange factor VAV (Vav), Rac, mitogen-activated protein kinase kinase 3, mitogen-activated protein kinase kinase 7, p38 MAP kinase (p38), c-Jun N-terminal kinase (JNK), growth factor receptor bound protein 2-associated protein 2 (Gab2), phosphatidylinositol-4,5-bisphosphate 3-kinase (PI3K), phospholipase C gamma (PLCγ), protein kinase C (PKC), 3-phosphoinositide dependent protein kinase 1 (PDK1), RAC serine/threonine-protein kinase (AKT), histamine, heparin, interleukin (IL)-3, IL-4, IL-13, IL-5, granulocyte-macrophage colony-stimulating factor (GM-CSF), tumor necrosis factor alpha (TNFα), leukotrienes (e.g., LTC4, LTD4 and LTE4) and prostaglandins (e.g., PDG2). In some embodiments, the FcεR antagonist is a BTK inhibitor (e.g., GDC-0853, acalabrutinib, GS-4059, spebrutinib, BGB-3111, or HM71224).

In some embodiments of any of the preceding methods, the therapy includes an IgE⁺ B cell depleting agent (e.g., an IgE⁺ B cell depleting antibody). In some embodiments, the IgE⁺ B cell depleting antibody is an anti-M1′ domain antibody. Any suitable anti-M1′ domain antibody may be used, for example, any anti-M1′ domain antibody described in International Patent Application Publication No. WO 2008/116149, which is incorporated herein by reference in its entirety. In some embodiments, the anti-M1′ domain antibody is afucosylated. In some embodiments, the anti-M1′ domain antibody is quilizumab or 47H4 (see, e.g., Brightbill et al. J. Clin. Invest. 120(6):2218-2229, 2010).

In some embodiments of any of the preceding methods, the therapy includes a mast cell or basophil depleting agent (e.g., a mast cell or basophil depleting antibody). In some embodiments, the antibody depletes mast cells. In other embodiments, the antibody depletes basophils. In yet other embodiments, the antibody depletes mast cells and basophils.

In some embodiments of any of the preceding methods, the therapy includes a PAR2 antagonist. Exemplary PAR2 antagonists include small molecule inhibitors (e.g., K-12940, K-14585, the peptide FSLLRY-NH2 (SEQ ID NO: 30), GB88, AZ3451, and AZ8838), soluble receptors, siRNAs, and anti-PAR2 antibodies (e.g., MAB3949 and Fab3949).

In some embodiments of any of the preceding methods, the therapy includes an IgE antagonist. In some embodiments, the IgE antagonist is an anti-IgE antibody. Any suitable anti-IgE antibody can be used. Exemplary anti-IgE antibodies include omalizumab (XOLAIR®), E26, E27, CGP-5101 (Hu-901), HA, ligelizumab, and talizumab. In some embodiments, the anti-IgE antibody includes one, two, three, four, five, or all six of the following six HVRs: (a) an HVR-H1 comprising the amino acid sequence of GYSWN (SEQ ID NO: 40); (b) an HVR-H2 comprising the amino acid sequence of SITYDGSTNYNPSVKG (SEQ ID NO: 41); (c) an HVR-H3 comprising the amino acid sequence of GSHYFGHWHFAV (SEQ ID NO: 42); (d) an HVR-L1 comprising the amino acid sequence of RASQSVDYDGDSYMN (SEQ ID NO: 43); (e) an HVR-L2 comprising the amino acid sequence of AASYLES (SEQ ID NO: 44); and (f) an HVR-L3 comprising the amino acid sequence of QQSHEDPYT (SEQ ID NO: 45). In some embodiments, the anti-IgE antibody includes (a) a VH domain comprising an amino acid sequence having at least 90%, at least 95%, or at least 99% sequence identity to the amino acid sequence of SEQ ID NO: 38; (b) a VL domain comprising an amino acid sequence having at least 90%, at least 95%, or at least 99% identity to the amino acid sequence of SEQ ID NO: 39; or (c) a VH domain as in (a) and a VL domain as in (b). In some embodiments, the VH domain comprises the amino acid sequence of SEQ ID NO: 38. In some embodiments, the VL domain comprises the amino acid sequence of SEQ ID NO: 39. In some embodiments, the VH domain comprises the amino acid sequence of SEQ ID NO: 38 and the VL domain comprises the amino acid sequence of SEQ ID NO: 39. Any of the anti-IgE antibodies described herein may be used in combination with any anti-tryptase antibody described herein, e.g., in Section VII below. In particular embodiments, the anti-IgE antibody is omalizumab (XOLAIR®).

In some embodiments of any of the preceding methods, the therapy includes a T_{H}2 pathway inhibitor. In some embodiments, the T_{H}2 pathway inhibitor inhibits any of the targets selected from interleukin-2-inducible T cell kinase (ITK), Bruton's tyrosine kinase (BTK), Janus kinase 1 (JAK1) (e.g., ruxolitinib, tofacitinib, oclacitinib, baricitinib, filgotinib, gandotinib, lestaurtinib, momelotinib, pacrinitib, upadacitinib, peficitinib, and fedratinib), GATA binding protein 3 (GATA3), IL-9 (e.g., MEDI-528), IL-5 (e.g., mepolizumab, CAS No. 196078-29-2; resilizumab), IL-13 (e.g., IMA-026, IMA-638 (also referred to as anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody)), IL-4 (e.g., AER-001, IL-4/IL-13 trap), OX40L, TSLP, IL-25, IL-33, and IgE (e.g., XOLAIR®, QGE-031; and MEDI-4212); and receptors such as: IL-9 receptor, IL-5 receptor (e.g., MEDI-563 (benralizumab, CAS No. 1044511-01-4)), IL-4 receptor alpha (e.g., AMG-317, AIR-645), IL-13 receptoralpha1 (e.g., R-1671) and IL-13 receptoralpha2, OX40, TSLP-R, IL-7Ralpha (a co-receptor for TSLP), IL-17RB (receptor for IL-25), ST2 (receptor for IL-33), CCR3, CCR4, CRTH2 (e.g., AMG-853, AP768, AP-761, MLN6095, ACT129968), FcεRI, FcεRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL-5, IL-3, and GM-CSF (e.g., TPI ASM8).

In some embodiments of any of the preceding methods, the asthma is persistent chronic severe asthma with acute events of worsening symptoms (exacerbations or flares) that can be life threatening. In some embodiments, the asthma is atopic (also known as allergic) asthma, non-allergic asthma (e.g., often triggered by infection with a respiratory virus (e.g., influenza, parainfluenza, rhinovirus, human metapneumovirus, and respiratory syncytial virus) or inhaled irritant (e.g., air pollutants, smog, diesel particles, volatile chemicals and gases indoors or outdoors, or even by cold dry air).

In some embodiments of any of the preceding methods, the asthma is intermittent or exercise-induced, asthma due to acute or chronic primary or second-hand exposure to “smoke” (typically cigarettes, cigars, or pipes), inhaling or “vaping” (tobacco, marijuana, or other such substances), or asthma triggered by recent ingestion of aspirin or related NSAIDS. In some embodiments, the asthma is mild, or corticosteroid naïve asthma, newly diagnosed and untreated asthma, or not previously requiring chronic use of inhaled topical or systemic steroids to control the symptoms (cough, wheeze, shortness of breath/breathlessness, or chest pain). In some embodiments, the asthma is chronic, corticosteroid resistant asthma, corticosteroid refractory asthma, or asthma uncontrolled on corticosteroids or other chronic asthma controller medications.

In some embodiments of any of the preceding methods, the asthma is moderate to severe asthma. In certain embodiments, the asthma is T_{H}2-high asthma. In some embodiments, the asthma is severe asthma. In some embodiments, the asthma is atopic asthma, allergic asthma, non-allergic asthma (e.g., due to infection and/or respiratory syncytial virus (RSV)), exercise-induced asthma, aspirin sensitive/exacerbated asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, or asthma uncontrolled on corticosteroids. In some embodiments, the asthma is T helper lymphocyte type 2 (T_{H}2) or type 2 (T_{H}2) high, or Type 2 (T2)-driven asthma. In some embodiments, the asthma is eosinophilic asthma. In some embodiments, the asthma is allergic asthma. In some embodiments, the individual has been determined to be Eosinophilic Inflammation Positive (EIP). See WO2015/061441. In some embodiments, the asthma is periostin-high asthma (e.g., having periostin level at least about any of 20 ng/ml, 25 ng/ml, or 50 ng/ml serum). In some embodiments, the asthma is eosinophil-high asthma (e.g., at least about any of 150, 200, 250, 300, 350, 400 eosinophil counts/ml blood). In certain embodiments, the asthma is T_{H}2-low asthma or non-T_{H}2-driven asthma. In some embodiments, the individual has been determined to be Eosinophilic Inflammation Negative (EIN). See WO2015/061441. In some embodiments, the asthma is periostin-low asthma (e.g., having periostin level less than about 20 ng/ml serum). In some embodiments, the asthma is eosinophil-low asthma (e.g., less than about 150 eosinophil counts/μl blood or less than about 100 eosinophil counts/μl blood).

For example, in particular embodiments of any of the preceding methods, the asthma is moderate to severe asthma. In some embodiments, the asthma is uncontrolled on a corticosteroid. In some embodiments, the asthma is T_{H}2 high asthma or T_{H}2 low asthma. In particular embodiments, the asthma is T_{H}2 high asthma.

It is to be understood that any of the methods of treating a patient described herein, e.g., in Section II of the Detailed Description of the Invention above, may be employed in embodiments where the method includes administering a therapy (e.g., a therapy comprising an agent selected from the group consisting of a tryptase antagonist, an Fc epsilon receptor (FcεR) antagonist, an IgE+ B cell depleting antibody, a mast cell or basophil depleting antibody, a protease activated receptor 2 (PAR2) antagonist, an IgE antagonist, and a combination thereof) to the patient. For example, in some embodiments, the method includes administering a therapy comprising an agent selected from the group consisting of a tryptase antagonist, an Fc epsilon receptor (FcεR) antagonist, an IgE+ B cell depleting antibody, a mast cell or basophil depleting antibody, a protease activated receptor 2 (PAR2) antagonist, and a combination thereof. In other embodiments, the method includes administering a therapy comprising an IgE antagonist.

### IV. Detection of Nucleic Acid Polymorphisms

In several embodiments, the methods of treatment and diagnosis provided by the invention involve determination of the genotype of a patient at one or more polymorphisms, for example, to determine a patient's active tryptase allele count. Detection techniques for evaluating nucleic acids for the presence of a polymorphism (e.g., a SNP (e.g., a c733 G>A SNP at TPSAB1, CTGCAGGCGGGCGTGGTCAGCTGGG[G/A]CGAGGGCTGTGCCCAGCCCAACCGG (SEQ ID NO: 36) (see also rs145402040) or an insertion (e.g., a c980_981insC mutation at TPSB2, CACACGGTCACCCTGCCCCCTGCCTCAGAGACCTTCCCCCCC (SEQ ID NO: 37), which is indicated by the bolded and underlined C nucleotide)) involve procedures well known in the field of molecular genetics. Many, but not all, of the methods involve amplification of nucleic acids. Ample guidance for performing amplification is provided in the art. Exemplary references include manuals such as Erlich, ed., PCR Technology: Principles and Applications for DNA Amplification, Freeman Press, 1992; Innis et al. eds., PCR Protocols: A Guide to Methods and Applications, Academic Press, 1990; Ausubel, ed., Current Protocols in Molecular Biology, 1994-1999, including supplemental updates through April 2004; and Sambrook et al. eds., Molecular Cloning, A Laboratory Manual, 2001. General methods for detection of single nucleotide polymorphisms are disclosed in Kwok, ed., Single Nucleotide Polymorphisms: Methods and Protocols, Humana Press, 2003.

Although the methods typically employ PCR steps, other amplification protocols may also be used. Suitable amplification methods include ligase chain reaction (see, e.g., Wu et al. Genomics 4:560-569, 1988); strand displacement assay (see, e.g., Walker et al. Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al. Proc. Natl. Acad. Sci. USA 86:1173-1177, 1989); and self-sustained sequence replication (3SR) (Guatelli et al. Proc. Natl. Acad. Sci. USA 87:1874-1878, 1990; WO 1992/08800). Alternatively, methods that amplify the probe to detectable levels can be used, such as Qβ-replicase amplification (Kramer et al. Nature 339:401-402, 1989; Lomeli et al. Clin. Chem. 35:1826-1831, 1989). A review of known amplification methods is provided, for example, by Abramson et al. Curr. Opin. Biotech. 4:41-47, 1993.

Detection of the genotype, haplotype, SNP, microsatellite, or other polymorphism of an individual can be performed using oligonucleotide primers and/or probes. Oligonucleotides can be prepared by any suitable method, usually chemical synthesis. Oligonucleotides can be synthesized using commercially available reagents and instruments. Alternatively, they can be purchased through commercial sources. Methods of synthesizing oligonucleotides are well known in the art (see, e.g., Narang et al. Meth. Enzymol. 68:90-99, 1979; Brown et al. Meth. Enzymol. 68:109-151, 1979; Beaucage et al. Tetra. Lett. 22:1859-1862, 1981; and the solid support method of U.S. Pat. No. 4,458,066). In addition, modifications to the above-described methods of synthesis may be used to desirably impact enzyme behavior with respect to the synthesized oligonucleotides. For example, incorporation of modified phosphodiester linkages (e.g., phosphorothioate, methylphosphonates, phosphoamidate, or boranophosphate) or linkages other than a phosphorous acid derivative into an oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

The genotype of an individual (e.g., a patient having a mast cell-mediated inflammatory disease (e.g., asthma)) can be determined using many detection methods that are well known in the art. Most assays entail one of several general protocols: sequencing, hybridization using allele-specific oligonucleotides, primer extension, allele-specific ligation, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microchips, fluorescence polarization assays, and MALDI-TOF (matrix assisted laser desorption ionization-time of flight) mass spectrometry. Two methods that can also be used are assays based on invasive cleavage with Flap nucleases and methodologies employing padlock probes.

Determination of the presence or absence of a particular allele is generally performed by analyzing a nucleic acid sample that is obtained from the individual to be analyzed. Often, the nucleic acid sample comprises genomic DNA. The genomic DNA is typically obtained from blood samples, but may also be obtained from other cells or tissues.

It is also possible to analyze RNA samples for the presence of polymorphic alleles. For example, mRNA can be used to determine the genotype of an individual at one or more polymorphic sites. In this case, the nucleic acid sample is obtained from cells in which the target nucleic acid is expressed, e.g., T helper-2 (Th2) cells and mast cells. Such an analysis can be performed by first reverse-transcribing the target RNA using, for example, a viral reverse transcriptase, and then amplifying the resulting cDNA; or using a combined high-temperature reverse-transcription-polymerase chain reaction (RT-PCR), as described in U.S. Pat. Nos. 5,310,652; 5,322,770; 5,561,058; 5,641,864; and 5,693,517.

The sample may be taken from a patient who is suspected of having, or is diagnosed as having a mast cell-mediated inflammatory disease (e.g., asthma), and hence is likely in need of treatment, or from a normal individual who is not suspected of having any disorder. For determination of genotypes, patient samples, such as those containing cells, or nucleic acids produced by these cells, may be used in the methods of the present invention. Bodily fluids or secretions useful as samples in the present invention include, e.g., blood, urine, saliva, stool, pleural fluid, lymphatic fluid, sputum, BAL, mucosal lining fluid (MLF) (e.g., MLF obtained by nasosorption or bronchosorption), ascites, prostatic fluid, cerebrospinal fluid (CSF), or any other bodily secretion or derivative thereof. The word blood is meant to include whole blood, plasma, serum, or any derivative of blood. Sample nucleic acid for use in the methods described herein can be obtained from any cell type or tissue of a subject. For example, a subject's bodily fluid (e.g., blood) can be obtained by known techniques. Alternatively, nucleic acid tests can be performed on dry samples (e.g., hair or skin).

The sample may be frozen, fresh, fixed (e.g., formalin fixed), centrifuged, and/or embedded (e.g., paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the genotype in the sample. Likewise, biopsies may also be subjected to post-collection preparative and storage techniques, e.g., fixation.

Frequently used methodologies for analysis of nucleic acid samples to detect the presence of polymorphisms such as SNPs or insertions which are useful in the present invention are briefly described below. However, any method known in the art can be used in the invention to detect the presence of single nucleotide substitutions.

a. DNA Sequencing and Single Base Extensions

Polymophisms, e.g., SNPs or insertions, can be detected by direct sequencing. Methods include e.g., dideoxy sequencing-based methods (e.g., Sanger sequencing) and other methods such as Maxam and Gilbert sequence (see, e.g., Sambrook and Russell, supra). In some embodiments, the sequencing approach is Sanger sequencing.

The sequencing approach may be a massively parallel sequencing approach (e.g., ILLUMINA® sequencing). Other detection methods include PYROSEQUENCING™ of oligonucleotide-length products. Such methods often employ amplification techniques such as PCR. For example, in pyrosequencing, a sequencing primer is hybridized to a single stranded, PCR-amplified, DNA template and incubated with the enzymes DNA polymerase, ATP sulfurylase, luciferase, and apyrase, and the substrates adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of APS. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated. Apyrase, a nucleotide degrading enzyme, continuously degrades unincorporated dNTPs and excess ATP. When degradation is complete, another dNTP is added.

In some embodiments, RNA sequencing (RNA-Seq), also referred to as whole transcriptome shotgun sequencing (WTSS), can be used to detect polymorphisms (e.g., SNPs or insertions). See, e.g., Wang et al. Nature Reviews Genetics 10:57-63, 2009.

Another similar method for characterizing SNPs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

b. Allele-Specific Hybridization

This technique, also commonly referred to as allele-specific oligonucleotide hybridization (ASO) (e.g., Stoneking et al. Am. J. Hum. Genet. 48:70-382, 1991; Saiki et al. Nature 324, 163-166, 1986; EP 235,726; and WO 1989/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g., 15-20 bases in length. The probes are designed to differentially hybridize to one variant versus another. Principles and guidance for designing such probe is available in the art. Hybridization conditions should be sufficiently stringent that there is a significant difference in hybridization intensity between alleles, and producing an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele can be determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing SNP sequences. After stringent hybridization and washing conditions, fluorescence intensity is measured for each SNP oligonucleotide.

In one embodiment, the nucleotide present at the polymorphic site is identified by hybridization under sequence-specific hybridization conditions with an oligonucleotide probe or primer exactly complementary to one of the polymorphic alleles in a region encompassing the polymorphic site. The probe or primer hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe or primer and the exactly complementary allelic sequence. Thus, oligonucleotides from about 10 to about 35 nucleotides in length, usually from about 15 to about 35 nucleotides in length, which are exactly complementary to an allele sequence in a region which encompasses the polymorphic site are within the scope of the invention.

In an alternative embodiment, the nucleotide present at the polymorphic site is identified by hybridization under sufficiently stringent hybridization conditions with an oligonucleotide substantially complementary to one of the SNP alleles in a region encompassing the polymorphic site, and exactly complementary to the allele at the polymorphic site. Because mismatches which occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe or primer and the target allele. Thus, oligonucleotides from about 10 to about 35 nucleotides in length, usually from about 15 to about 35 nucleotides in length, which are substantially complementary to an allele sequence in a region which encompasses the polymorphic site, and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-oligonucleotide assay format, probes or primers for each target are immobilized on a single solid support. Hybridizations are carried out simultaneously by contacting the solid support with a solution containing target DNA. As all hybridizations are carried out under identical conditions, the hybridization conditions cannot be separately optimized for each probe or primer. The incorporation of mismatches into a probe or primer can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe or primer, can be selected empirically using the guidance provided herein and well known in the art. The use of oligonucleotide probes or primers to detect single base pair differences in sequence is described in, for example, Conner et al. Proc. Natl. Acad. Sci. USA 80:278-282, 1983, and U.S. Pat. Nos. 5,468,613 and 5,604,099.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. Oligonucleotides between about 15 and about 35 nucleotides in length are often used for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. For discrimination of a single base pair change in target sequence, the probe sequence is selected which hybridizes to the target sequence such that the polymorphic site occurs in the interior region of the probe.

The above criteria for selecting a probe sequence that hybridizes to a specific allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA. A reverse line-blot detection assay is described in the example.

An allele-specific probe that is specific for one of the polymorphism variants is often used in conjunction with the allele-specific probe for the other polymorphism variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized polymorphisms. WO 95/11995 also describes subarrays that are optimized for detection of variant forms of a pre-characterized polymorphism. Such a subarray can be used in detecting the presence of the polymorphisms described herein.

c. Allele-Specific Primers

Polymorphisms such as SNPs or insertions are also commonly detected using allele-specific amplification or primer extension methods. These reactions typically involve use of primers that are designed to specifically target a polymorphism via a mismatch at the 3′-end of a primer. The presence of a mismatch affects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, for example, WO 93/22456 and U.S. Pat. Nos. 5,137,806; 5,595,890; 5,639,611; and 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g., in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reactions that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′-terminus of the oligonucleotide, which mismatches occur at non-polymorphic sites in both allele sequences.

d. Detectable Probes

i) 5′-Nuclease Assay Probes

Genotyping can also be performed using a “TAQMAN®” or “5′-nuclease assay,” as described in U.S. Pat. Nos. 5,210,015; 5,487,972; and 5,804,375; and Holland et al. Proc. Natl. Acad. Sci. USA 88:7276-7280, 1988. In the TAQMAN® assay, labeled detection probes that hybridize within the amplified region are added during the amplification reaction. The probes are modified so as to prevent the probes from acting as primers for DNA synthesis. The amplification is performed using a DNA polymerase having 5′- to 3′-exonuclease activity. During each synthesis step of the amplification, any probe which hybridizes to the target nucleic acid downstream from the primer being extended is degraded by the 5′- to 3′-exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNP alleles. Alternatively, the method can be performed using an allele-specific primer and a labeled probe that binds to amplified product.

Any method suitable for detecting degradation product can be used in a 5′-nuclease assay. Often, the detection probe is labeled with two fluorescent dyes, one of which is capable of quenching the fluorescence of the other dye. The dyes are attached to the probe, usually one attached to the 5′-terminus and the other is attached to an internal site, such that quenching occurs when the probe is in an unhybridized state and such that cleavage of the probe by the 5′- to 3′-exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673 describe alternative methods for detecting the degradation of probe which occurs concomitant with amplification.

ii) Secondary Structure Probes

Probes detectable upon a secondary structural change are also suitable for detection of a polymorphism, including SNPs. Exemplified secondary structure or stem-loop structure probes include molecular beacons or SCORPION® primer/probes. Molecular beacon probes are single-stranded oligonucleic acid probes that can form a hairpin structure in which a fluorophore and a quencher are usually placed on the opposite ends of the oligonucleotide. At either end of the probe short complementary sequences allow for the formation of an intramolecular stem, which enables the fluorophore and the quencher to come into close proximity. The loop portion of the molecular beacon is complementary to a target nucleic acid of interest. Binding of this probe to its target nucleic acid of interest forms a hybrid that forces the stem apart. This causes a conformation change that moves the fluorophore and the quencher away from each other and leads to a more intense fluorescent signal. Molecular beacon probes are, however, highly sensitive to small sequence variation in the probe target (see, e.g., Tyagi et al. Nature Biotech. 14:303-308, 1996; Tyagi et al. Nature Biotech. 16:49-53, 1998; Piatek et al. Nature Biotech. 16: 359-363, 1998; Marras et al. Genetic Analysis: Biomolecular Engineering 14:151-156,1999; Tapp et al, BioTechniques 28: 732-738, 2000). A SCORPION® primer/probe comprises a stem-loop structure probe covalently linked to a primer.

e. Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g., Erlich, ed., PCR Technology, Principles and Applications for DNA Amplification, W. H. Freeman and Co., 1992).

Distinguishing of microsatellite polymorphisms can be done using capillary electrophoresis. Capillary electrophoresis conveniently allows identification of the number of repeats in a particular microsatellite allele. The application of capillary electrophoresis to the analysis of DNA polymorphisms is well known to those in the art (see, for example, Szantai et al. J Chromatogr A. 1079(1-2):41-9, 2005; Bjorheim et al. Electrophoresis 26(13):2520-30, 2005 and Mitchelson, Mol. Biotechnol. 24(1):41-68, 2003).

The identity of the allelic variant may also be obtained by analyzing the movement of a nucleic acid comprising the polymorphic region in polyacrylamide gels containing a gradient of denaturant, which is assayed using denaturing gradient gel electrophoresis (DGGE) (see, e.g., Myers et al. Nature 313:495-498, 1985). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing agent gradient to identify differences in the mobility of control and sample DNA (see, e.g., Rosenbaum et al. Biophys. Chem. 265:1275, 1987).

f. Single-Strand Conformation Polymorphism Analysis

Alleles of target sequences can be differentiated using single-strand conformation polymorphism analysis, which identifies base differences by alteration in electrophoretic migration of single stranded PCR products, as described, e.g., in Orita et al. Proc. Nat. Acad. Sci. 86, 2766-2770, 1989; Cotton Mutat. Res. 285:125-144, 1993; and Hayashi Genet. Anal. Tech. Appl. 9:73-79, 1992. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (see, e.g., Keen et al. Trends Genet. 7:5-10, 1991).

SNP detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g., ³²P, electron-dense reagents, enzyme, such as peroxidase or alkaline phosphatase, biotin, or haptens and proteins for which antisera or monoclonal antibodies are available. Labeling techniques are well known in the art (see, e.g., Current Protocols in Molecular Biology, supra; Sambrook et al., supra).

g. Additional Methods to Determine the Genotype of an Individual at Polymorphisms

DNA microarray technology, e.g., DNA chip devices, high-density microarrays for high-throughput screening applications, and lower-density microarrays may be used. Methods for microarray fabrication are known in the art and include various inkjet and microjet deposition or spotting technologies and processes, in situ or on-chip photolithographic oligonucleotide synthesis processes, and electronic DNA probe addressing processes. DNA microarray hybridization applications have been successfully applied in the areas of gene expression analysis and genotyping for point mutations, single nucleotide polymorphisms (SNPs), and short tandem repeats (STRs). Additional methods include interference RNA microarrays and combinations of microarrays and other methods such as laser capture microdissection (LCM), comparative genomic hybridization (CGH), array CGH, and chromatin immunoprecipitation (ChIP). See, e.g., He et al. Adv. Exp. Med. Biol. 593:117-133, 2007 and Heller Annu. Rev. Biomed. Eng. 4:129-153, 2002.

In some embodiments, protection from cleavage agents (such as a nuclease, hydroxylamine or osmium tetroxide and with piperidine) can be used to detect mismatched bases in RNA/RNA, DNA/DNA, or RNA/DNA heteroduplexes (see, e.g., Myers et al. Science 230:1242, 1985). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene with a sample nucleic acid, e.g., RNA or DNA, obtained from a tissue sample. The double-stranded duplexes are treated with an agent that cleaves single-stranded regions of the duplex, such as duplexes formed based on base pair mismatches between the control and sample strands. For instance, RNA/DNA duplexes can be treated with RNase and DNA/DNA hybrids can be treated with S1 nuclease to enzymatically digest the mismatched regions. Alternatively, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. Proc. Natl. Acad. Sci. USA 85:4397-4401, 1988; Saleeba et al. Meth. Enzymol. 217:286-295, 1992.

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In another embodiment, identification of the allelic variant is carried out using an oligonucleotide ligation assay (OLA), as described, for example, in U.S. Pat. No. 4,998,617 and Laridegren et al. Science 241:1077-1080, 1988. The OLA protocol uses two oligonucleotides which are designed to be capable of hybridizing to abutting sequences of a single strand of a target. One of the oligonucleotides is linked to a separation marker, e.g., by biotinylation, and the other is detectably labeled. If the precise complementary sequence is found in a target molecule, the oligonucleotides will hybridize such that their termini abut, and create a ligation substrate. Ligation then permits the labeled oligonucleotide to be recovered using avidin or another biotin ligand. Also known in the art is a nucleic acid detection assay that combines attributes of PCR and OLA (see, e.g., Nickerson et al. Proc. Natl. Acad. Sci. USA 87:8923-8927, 1990). In this method, PCR is used to achieve the exponential amplification of target DNA, which is then detected using OLA.

A single base polymorphism can be detected by using a specialized exonuclease-resistant nucleotide, as described, for example, in U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

A solution-based method may also be used for determining the identity of the nucleotide of the polymorphic site (see, e.g., WO 1991/02087). As above, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method that may be used is described in WO 92/15712. This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. The method is usually a heterogeneous phase assay, in which the primer or the target molecule is immobilized to a solid phase.

Many other primer-guided nucleotide incorporation procedures for assaying polymorphic sites in DNA have been described (Komher et al. Nucl. Acids. Res. 17:7779-7784, 1989; Sokolov Nucl. Acids Res. 18:3671, 1990; Syvanen et al. Genomics 8:684-692, 1990; Kuppuswamy et al. Proc. Natl. Acad. Sci. USA 88:1143-1147, 1991; Prezant et al. Hum. Mutat. 1:159-164, 1992; Ugozzoli et al. GATA 9:107-112, 1992; Nyren et al. Anal. Biochem. 208:171-175, 1993). These methods all rely on the incorporation of labeled deoxynucleotides to discriminate between bases at a polymorphic site.

### V. Determination of the Expression Level of Biomarkers

The therapeutic and diagnostic methods of the invention can involve determination of the expression level of one or more biomarkers (e.g., tryptase). The determination of the level of biomarkers can be performed by any of the methods known in the art or described below.

Expression of biomarkers described herein (e.g., tryptase) can be detected using any method known in the art. For example, tissue or cell samples from mammals can be conveniently assayed for, e.g., mRNAs or DNAs of a biomarker of interest using Northern, dot-blot, or PCR analysis, array hybridization, RNase protection assay, or using DNA SNP chip microarrays, which are commercially available, including DNA microarray snapshots. For example, real-time PCR (RT-PCR) assays such as quantitative PCR assays are well known in the art. In an illustrative embodiment of the invention, a method for detecting mRNA of a biomarker of interest (e.g., tryptase) in a biological sample comprises producing cDNA from the sample by reverse transcription using at least one primer; amplifying the cDNA so produced; and detecting the presence of the amplified cDNA. In addition, such methods can include one or more steps that allow one to determine the levels of mRNA in a biological sample (e.g., by simultaneously examining the levels a comparative control mRNA sequence of a “housekeeping” gene such as an actin family member). Optionally, the sequence of the amplified cDNA can be determined.

Other methods that can be used to detect nucleic acids, for use in the invention, involve high-throughput RNA sequence expression analysis, including RNA-based genomic analysis, such as, for example, RNASeq.

In one specific embodiment, expression of a biomarker (e.g., tryptase) can be performed by RT-PCR technology. Probes used for PCR may be labeled with a detectable marker, such as, for example, a radioisotope, fluorescent compound, bioluminescent compound, a chemiluminescent compound, metal chelator, or enzyme. Such probes and primers can be used to detect the presence of an expressed biomarker in a sample. As will be understood by the skilled artisan, a great many different primers and probes may be prepared based on the sequences provided in herein and used effectively to amplify, clone and/or determine the presence and/or levels of a biomarker.

Other methods include protocols that examine or detect mRNAs of a biomarker (e.g., tryptase), in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 2001/75166). See, for example, U.S. Pat. Nos. 5,700,637, 5,445,934, and 5,807,522, Lockart, Nat. Biotech. 14:1675-1680, 1996; and Cheung et al. Nat. Genet. 21(Suppl):15-19, 1999 for a discussion of array fabrication.

In addition, the DNA profiling and detection method utilizing microarrays described in European Patent EP 1753878 may be employed. This method rapidly identifies and distinguishes between different DNA sequences utilizing short tandem repeat (STR) analysis and DNA microarrays. In an embodiment, a labeled STR target sequence is hybridized to a DNA microarray carrying complementary probes. These probes vary in length to cover the range of possible STRs. The labeled single-stranded regions of the DNA hybrids are selectively removed from the microarray surface utilizing a post-hybridization enzymatic digestion. The number of repeats in the unknown target is deduced based on the pattern of target DNA that remains hybridized to the microarray.

One example of a microarray processor is the Affymetrix GENECHIP® system, which is commercially available and comprises arrays fabricated by direct synthesis of oligonucleotides on a glass surface. Other systems may be used as known to one skilled in the art.

Many references are available to provide guidance in applying the above techniques (Kohler et al. Hybridoma Techniques, Cold Spring Harbor Laboratory, 1980; Tijssen, Practice and Theory of Enzyme Immunoassays, Elsevier, 1985; Campbell, Monoclonal Antibody Technology, Elsevier, 1984; Hurrell, Monoclonal Hybridoma Antibodies: Techniques and Applications, CRC Press, 1982; and Zola, Monoclonal Antibodies: A Manual of Techniques, pp. 147-158, CRC Press, Inc., 1987). Northern blot analysis is a conventional technique well known in the art and is described, for example, in Sambrook et al, supra. Typical protocols for evaluating the status of genes and gene products are found, for example in Ausubel et al., supra. As to detection of protein biomarkers, various protein assays are available including, for example, antibody-based methods as well as mass spectroscopy and other similar means known in the art. In the case of antibody-based methods, for example, the sample may be contacted with an antibody specific for the biomarker (e.g., tryptase) under conditions sufficient for an antibody-biomarker complex to form, and then detecting the complex. Detection of the presence of the protein biomarker may be accomplished in a number of ways, such as by Western blotting (with or without immunoprecipitation), 2-dimensional sodium dodecyl sulfate polyacrylamide gel electrophoresis (SDS-PAGE), immunoprecipitation, fluorescence activated cell sorting (FACS™), flow cytometry, and enzyme-linked immunosorbent assay (ELISA) procedures for assaying a wide variety of tissues and samples, including plasma or serum. A wide range of immunoassay techniques using such an assay format are available, see, e.g., U.S. Pat. Nos. 4,016,043; 4,424,279; and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested is brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed, dried, and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent molecules.

In the case of an enzyme immunoassay (EIA), an enzyme is conjugated to the second antibody, generally by means of glutaraldehyde or periodate. As will be readily recognized, however, a wide variety of different conjugation techniques exist, which are readily available to the skilled artisan. Examples of commonly used enzymes suitable for methods of the present invention include horseradish peroxidase, glucose oxidase, beta-galactosidase, and alkaline phosphatase. The substrates to be used with the specific enzymes are generally chosen for the production, upon hydrolysis by the corresponding enzyme, of a detectable color change. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker (e.g., tryptase) which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

In some embodiments, the level of active tryptase in a sample (e.g., blood (e.g., serum or plasma), BAL, or MLF) can be determined using an active tryptase ELISA assay, for example, as described in Example 6 of U.S. Provisional Patent Application No. 62/457,722. The concentration of human active tryptase (tetramer) can be determined by an ELISA assay. Briefly, a monoclonal antibody clone recognizing human tryptase is utilized as the capture antibody (e.g., the monoclonal antibody B12 described in Fukuoka et al. supra, or the E88AS antibody clone). Any suitable antibody that binds human tryptase can be used. Recombinant human active tryptase beta 1 is purified and used as the source material for preparation of assay standards. Assay standards, controls, and diluted samples were incubated with 500 μg/ml soybean trypsin inhibitor (SBTI; Sigma Cat. No. 10109886001) for 10 min and then labeled with an activity-based probe (ABP) (G0353816) for 1 h. A small molecule tryptase inhibitor (G02849855) is added for 20 min to stop ABP labeling. Depending on the capture antibody used in the assay, this mixture may be incubated with an anti-human tryptase antibody that is capable of dissociating the tryptase tetramer (e.g., hu31A.v11 or B12) before being added to the ELISA plate with capture antibody for 1 h, washed with 1× phospho-buffered saline—TWEEN® (PBST), and incubated with SA-HRP reagent (streptavidin-conjugated horseradish peroxidase, General Electric (GE) catalog number RPN4401V) for 2 h. A colorimetric signal is generated by applying HRP substrate, tetramethylbenzidine (TMB), and the reaction is stopped by adding phosphoric acid. The plates are read on a plate reader (e.g., a SpectraMax® M5 plate reader) using 450 nm for detection absorbance and 650 nm for reference absorbance. A similar assay can be conducted to determine the level of active cynomolgus monkey (cyno) tryptase in a sample (e.g., blood (e.g., serum or plasma), BAL, or MLF), for example, using antibody clone 13G6 as the capture antibody.

In some embodiments, the level of total tryptase in a sample (e.g., blood (e.g., serum or plasma), BAL, or MLF) can be determined using a total tryptase ELISA assay, for example, as described in Example 6 of U.S. Provisional Patent Application No. 62/457,722. Briefly, the concentration of human total tryptase can be determined by an ELISA assay. An antibody recognizing human tryptase is utilized as the capture antibody (e.g., antibody clone B12). A monoclonal antibody recognizing human tryptase is utilized as the detection antibody (e.g., antibody clone E82AS). Recombinant human active tryptase beta 1 is purified and used as the source material for preparation of assay standards. Depending on the capture antibody used in the assay, this mixture may be incubated with an anti-human tryptase antibody that is capable of dissociating the tryptase tetramer (e.g., hu31A.v11 or B12) before being added to the ELISA plate with capture antibody for 2 h and then washed with 1×PBST. The biotinylated detection antibody is added for 1 h. Next, SA-HRP reagent is added for 1 h. A colorimetric signal is generated by applying TMB, and the reaction is stopped by adding phosphoric acid. The plates are read on a plate reader (e.g., a SpectraMax® M5 plate reader) using 450 nm for detection absorbance and 650 nm for reference absorbance. A similar assay can be conducted to determine the level of total cynomolgus monkey (cyno) tryptase in a sample (e.g., blood (e.g., serum or plasma), BAL, or MLF), for example, using antibody clone 13G6 as the capture antibody and antibody clone E88AS as the detection assay.

In some embodiments, an exemplary reference level for total tryptase in blood (e.g., serum or plasma) may be about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, or about 10 ng/ml. For example, in some embodiments, an exemplary reference level for total tryptase in plasma is about 3 ng/ml. In another example, in some embodiments, an exemplary reference level for total tryptase in serum is about 4 ng/ml. For example, in some embodiments, a subject may have a total tryptase level that is at or above a reference level if the subject's total tryptase level (e.g., in blood (e.g., serum or plasma) is about 1 ng/ml or higher, about 2 ng/ml or higher, about 3 ng/ml or higher, about 4 ng/ml or higher, about 5 ng/ml or higher, about 6 ng/ml or higher, about 7 ng/ml or higher, about 8 ng/ml or higher, about 9 ng/ml or higher, or about 10 ng/ml or higher. For example, in some embodiments, a subject may have a total tryptase level that is at or above a reference level if the subject's total plasma tryptase level is 3 ng/ml or higher. In another example, in some embodiments, a subject may have a total tryptase level that is at or above a reference level if the subject's total serum tryptase level is 4 ng/ml or higher.

In some embodiments of the present invention, a Total Periostin Assay, as described in International Patent Application Publication No. WO 2012/083132, which is incorporated herein by reference in its entirety, is used to determine the level of periostin in a sample derived from the patient. For example, a periostin capture ELISA assay that is very sensitive (sensitivity of approximately 1.88 ng/ml) referred to as the E4 assay in WO 2012/083132 can be used. The antibodies recognize periostin isoforms 1-4 (SEQ ID NOs:5-8 of WO 2012/083132) at nanomolar affinity. In other embodiments, the ELECSYS® periostin assay described in WO 2012/083132 can be used to determine the level of periostin in a sample derived from the patient.

In some embodiments, an exemplary reference level for periostin levels is 20 ng/ml, for example, when using the E4 assay described above. For instance, when using the E4 assay, a patient may have a periostin level at or greater than a reference level if the patient's periostin level (e.g., in serum or plasma) is 20 ng/ml or higher, 21 ng/ml or higher, 22 ng/ml or higher, 23 ng/ml or higher, 24 ng/ml or higher, 25 ng/ml or higher, 26 ng/ml or higher, 27 ng/ml or higher, 28 ng/ml or higher, 29 ng/ml or higher, 30 ng/ml or higher, 31 ng/ml or higher, 32 ng/ml or higher, 33 ng/ml or higher, 34 ng/ml or higher, 35 ng/ml or higher, 36 ng/ml or higher, 37 ng/ml or higher, 38 ng/ml or higher, 39 ng/ml or higher, 40 ng/ml or higher, 41 ng/ml or higher, 42 ng/ml or higher, 43 ng/ml or higher, 44 ng/ml or higher, 45 ng/ml or higher, 46 ng/ml or higher, 47 ng/ml or higher, 48 ng/ml or higher, 49 ng/ml or higher, 50 ng/ml or higher, 51 ng/ml or higher, 52 ng/ml or higher, 53 ng/ml or higher, 54 ng/ml or higher, 55 ng/ml or higher, 56 ng/ml or higher, 57 ng/ml or higher, 58 ng/ml or higher, 59 ng/ml or higher, 60 ng/ml or higher, 61 ng/ml or higher, 62 ng/ml or higher, 63 ng/ml or higher, 64 ng/ml or higher, 65 ng/ml or higher, 66 ng/ml or higher, 67 ng/ml or higher, 68 ng/ml or higher, 69 ng/ml or higher or 70 ng/ml or higher.

When using the E4 assay, a patient may have a periostin level at or below a reference level if the patient's periostin level (e.g., in serum or plasma) is 20 ng/ml or lower, 19 ng/ml or lower, 18 ng/ml or lower, 17 ng/ml or lower, 16 ng/ml or lower, 15 ng/ml or lower, 14 ng/ml or lower, 13 ng/ml or lower, 12 ng/ml or lower, 11 ng/ml or lower, 10 ng/ml or lower, 9 ng/ml or lower, 8 ng/ml or lower, 7 ng/ml or lower, 6 ng/ml or lower, 5 ng/ml or lower, 4 ng/ml or lower, 3 ng/ml or lower, 2 ng/ml or lower, or 1 ng/ml or lower.

In other embodiments, an exemplary reference level for periostin levels (e.g., in serum or plasma) is 50 ng/ml, for example, when using the ELECSYS® periostin assay described above. For instance, when using the ELECSYS® periostin assay, a patient may have a periostin level at or greater than a reference level if the patient's periostin level is 50 ng/ml or higher, 51 ng/ml or higher, 52 ng/ml or higher, 53 ng/ml or higher, 54 ng/ml or higher, 55 ng/ml or higher, 56 ng/ml or higher, 57 ng/ml or higher, 58 ng/ml or higher, 59 ng/ml or higher, 60 ng/ml or higher, 61 ng/ml or higher, 62 ng/ml or higher, 63 ng/ml or higher, 64 ng/ml or higher, 65 ng/ml or higher, 66 ng/ml or higher, 67 ng/ml or higher, 68 ng/ml or higher, 69 ng/ml or higher, 70 ng/ml or higher, 71 ng/ml or higher, 72 ng/ml or higher, 73 ng/ml or higher, 74 ng/ml or higher, 75 ng/ml or higher, 76 ng/ml or higher, 77 ng/ml or higher, 78 ng/ml or higher, 79 ng/ml or higher, 80 ng/ml or higher, 81 ng/ml or higher, 82 ng/ml or higher, 83 ng/ml or higher, 84 ng/ml or higher, 85 ng/ml or higher, 86 ng/ml or higher, 87 ng/ml or higher, 88 ng/ml or higher, 89 ng/ml or higher, 90 ng/ml or higher, 91 ng/ml or higher, 92 ng/ml or higher, 93 ng/ml or higher, 94 ng/ml or higher, 95 ng/ml or higher, 96 ng/ml or higher, 97 ng/ml or higher, 98 ng/ml or higher, or 99 ng/ml or higher.

When using the ELECSYS® periostin assay, a patient may have a periostin level at or below a reference level if the patient's periostin level (e.g., in serum or plasma) is 50 ng/ml or lower, 49 ng/ml or lower, 48 ng/ml or lower, 47 ng/ml or lower, 46 ng/ml or lower, 45 ng/ml or lower, 44 ng/ml or lower, 43 ng/ml or lower, 42 ng/ml or lower, 41 ng/ml or lower, 40 ng/ml or lower, 39 ng/ml or lower, 38 ng/ml or lower, 37 ng/ml or lower, 36 ng/ml or lower, 35 ng/ml or lower, 34 ng/ml or lower, 33 ng/ml or lower, 32 ng/ml or lower, 31 ng/ml or lower, 30 ng/ml or lower, 29 ng/ml or lower, 28 ng/ml or lower, 27 ng/ml or lower, 26 ng/ml or lower, 25 ng/ml or lower, 24 ng/ml or lower, 23 ng/ml or lower, 22 ng/ml or lower, 21 ng/ml or lower, 20 ng/ml or lower, 19 ng/ml or lower, 18 ng/ml or lower, 17 ng/ml or lower, 16 ng/ml or lower, 15 ng/ml or lower, 14 ng/ml or lower, 13 ng/ml or lower, 12 ng/ml or lower, 11 ng/ml or lower, 10 ng/ml or lower, 9 ng/ml or lower, 8 ng/ml or lower, 7 ng/ml or lower, 6 ng/ml or lower, 5 ng/ml or lower, 4 ng/ml or lower, 3 ng/ml or lower, 2 ng/ml or lower, or 1 ng/ml or lower.

### VI. Kits

For use in detection of the presence and/or expression level of biomarkers (e.g., tryptase), kits or articles of manufacture are also provided by the invention. Such kits can be used for determining whether a patient having a mast-cell mediated inflammatory disorder (e.g., asthma) is likely to respond to a therapy, for example, a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof (e.g., a tryptase antagonist and an IgE antagonist), or a therapy comprising an IgE antagonist or an Fc epsilon receptor (FcεR) antagonist, and/or for assessing or monitoring a response of a patient having asthma to treatment with a therapy. In some embodiments, the kits can be used to determine a patient's active tryptase allele count. In other embodiments, the kits can be used to determine the expression level of tryptase (e.g., active or total tryptase) in a sample from a patient. Such kits can be used for carrying out any of the methods of the invention.

For example, the invention features a kit for identifying a patient having a mast cell-mediated inflammatory disease who is likely to respond to a mast cell-directed therapy (e.g., a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof (e.g., a tryptase antagonist and an IgE antagonist)), the kit including: (a) reagents for determining the patient's active tryptase allele count or for determining the expression level of tryptase in a sample from the patient; and, optionally, (b) instructions for using the reagents to identify a patient having a mast cell-mediated inflammatory disease who is likely to respond to a mast cell-directed therapy (e.g., a therapy comprising an agent selected from the group consisting of a tryptase antagonist, an IgE antagonist, an FcεR antagonist, an IgE⁺ B cell depleting antibody, a mast cell or basophil depleting antibody, a PAR2 antagonist, and a combination thereof (e.g., a tryptase antagonist and an IgE antagonist)). In some embodiment, the kit includes reagents for determining the patient's active tryptase allele count. In other embodiments, the kit includes reagents for determining the expression level of tryptase in a sample from the patient.

In another example, the invention features a kit for identifying a patient having a mast cell-mediated inflammatory disease who is likely to respond to a therapy comprising an IgE antagonist or an FcεR antagonist that includes (a) reagents for determining the patient's active tryptase allele count or for determining the expression level of tryptase in a sample from the patient; and, optionally, (b) instructions for using the reagents to identify a patient having a mast cell-mediated inflammatory disease who is likely to respond to a therapy comprising an IgE antagonist or an FcεR antagonist.

Any suitable reagents for determining the patient's active tryptase allele count or for determining the expression level of tryptase can be used in any of the preceding kits, including, for example, oligonucleotides, polypeptides (e.g., antibodies), and the like.

In some embodiments, the kit further comprises reagents for determining the level of a Type 2 biomarker in a sample from the patient.

For example, in some embodiments, the reagent comprises an oligonucleotide. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, e.g., less than 1 kb from the polymorphism. Specific oligonucleotides are capable of hybridizing to a sequence, and under suitable conditions will not bind to a sequence differing by a single nucleotide.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In some embodiments, the surface is silica or glass. In some embodiments, the surface is a metal electrode.

In other embodiments, the reagent for determining the expression level of tryptase may be a polypeptide, for example, an antibody. In some embodiments, the antibody may be detectably labeled.

Yet other kits of the invention comprise at least one reagent necessary to perform the assay. For example, the kit can comprise an enzyme. Alternatively the kit can comprise a buffer or any other necessary reagent. The kits can include all or some of the positive controls, negative controls, reagents, primers, sequencing markers, probes, and antibodies described herein for determining the patient's active tryptase allele count or determining the expression level of tryptase in a sample from the patient.

Any of the preceding kits may comprise a carrier being compartmentalized to receive in close confinement one or more containers such as vials, tubes, and the like, each of the containers comprising one of the separate elements to be used in the method. For example, one of the containers may comprise a probe that is or can be detectably labeled. Such probe may be an antibody or oligonucleotide specific for a protein or message, respectively. Where the kit utilizes nucleic acid hybridization to detect the target nucleic acid, the kit may also have containers containing nucleotide(s) for amplification of the target nucleic acid sequence and/or a container comprising a reporter, such as a biotin-binding protein (e.g., avidin or streptavidin) bound to a reporter molecule, such as an enzymatic, florescent, or radioisotope label.

Such kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the invention have a number of embodiments. A typical embodiment is a kit comprising a container, a label on said container, and a composition contained within said container, wherein the composition includes a primary antibody that binds to a protein biomarker (e.g., tryptase), and the label on said container indicates that the composition can be used to evaluate the presence of such proteins in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label, e.g., an enzymatic label.

Another embodiment is a kit comprising a container, a label on said container, and a composition contained within said container, wherein the composition includes one or more polynucleotides that hybridize to a complement of a biomarker (e.g., tryptase) under stringent conditions, and the label on said container indicates that the composition can be used to evaluate the presence of a biomarker (e.g., tryptase) in a sample, and wherein the kit includes instructions for using the polynucleotide(s) for evaluating the presence of the biomarker RNA or DNA in a particular sample type.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In further specific embodiments, for antibody-based kits, the kit can comprise, for example: (1) a first antibody (e.g., attached to a solid support) that binds to a biomarker protein (e.g., tryptase); and, optionally, (2) a second, different antibody that binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a tryptase gene (e.g., TPSAB1 or TPSB2), and/or a nucleic acid sequence encoding a biomarker protein (e.g., tryptase) or (2) a pair of primers useful for amplifying a biomarker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container and all of the various containers can be within a single package, along with instructions for interpreting the results of the assays performed using the kit.

Any of the preceding kits may further include one or more therapeutic agents, including any of the tryptase antagonists, FcεR antagonists, IgE⁺ B cell depleting antibodies, mast cell or basophil depleting antibodies, PAR2 antagonists, IgE antagonists, and combinations thereof (e.g., a tryptase antagonist and an IgE antagonist), and/or additional therapeutic agents described herein.

### VII. Compositions and Pharmaceutical Formulations

In one aspect, the invention is based, in part, on the discovery that biomarkers of the invention (e.g., a patient's active tryptase allele count and/or the expression level of tryptase) can be used to identify patients having a mast cell-mediated inflammatory disease are likely to respond to a therapy (e.g., a therapy comprising an agent selected from the group consisting of a tryptase antagonist, an Fc epsilon receptor (FcεR) antagonist, an IgE⁺ B cell depleting antibody, a mast cell or basophil depleting antibody, a protease activated receptor 2 (PAR2) antagonist, an IgE antagonist, and a combination thereof (e.g., a tryptase antagonist and an IgE antagonist)). These agents, and combinations thereof, are useful for the treatment of mast cell-mediated inflammatory diseases, e.g., as part of any of the methods described herein, for example, in Sections II and III above. In some embodiments, the therapy is a mast cell-directed therapy. Any suitable tryptase antagonist (e.g., anti-tryptase antibody), Fc epsilon receptor (FcεR) antagonist, IgE⁺ B cell depleting antibody, mast cell or basophil depleting antibody, protease activated receptor 2 (PAR2) antagonist, and/or IgE antagonist can be used in the methods and assays described herein. Non-limiting examples suitable for use in the methods and assays of the invention are described further below.

A. Antibodies

Any suitable antibody can be used in the methods described herein, for example, anti-tryptase antibodies, anti-FcεR antibodies, IgE⁺ B cell depleting antibodies, mast cell or basophil depleting antibodies, anti-PAR2 antibodies, and/or anti-IgE antibodies. It is expressly contemplated that such anti-tryptase antibodies, anti-FcεR antibodies, IgE⁺ B cell depleting antibodies, mast cell or basophil depleting antibodies, anti-PAR2 antibodies, and/or anti-IgE antibodies for use in any of the embodiments enumerated above may have any of the features, singly or in combination, described in Sections a-c and 1-7 below.

a. Anti-Tryptase Antibodies

Any suitable anti-tryptase antibody can be used in the methods of the invention. For example, the anti-tryptase antibody may be any anti-tryptase antibody described in U.S. Provisional Patent Application No. 62/457,722, which is incorporated herein by reference in its entirety.

In some embodiments, the anti-tryptase antibody (e.g., the anti-tryptase beta antibody) can include at least one, at least two, at least three, at least four, at least five, or all six hypervariable regions (HVRs) selected from (a) an HVR-H1 comprising the amino acid sequence of DYGMV (SEQ ID NO: 1); (b) an HVR-H2 comprising the amino acid sequence of FISSGSSTVYYADTMKG (SEQ ID NO: 2); (c) an HVR-H3 comprising the amino acid sequence of RNYDDWYFDV (SEQ ID NO: 3); (d) an HVR-L1 comprising the amino acid sequence of SASSSVTYMY (SEQ ID NO: 4); (e) an HVR-L2 comprising the amino acid sequence of RTSDLAS (SEQ ID NO: 5); and (f) an HVR-L3 comprising the amino acid sequence of QHYHSYPLT (SEQ ID NO: 6), or a combination of one or more of the above HVRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 1-6. For example, in some embodiments, the anti-tryptase antibody includes (a) an HVR-H1 comprising the amino acid sequence of DYGMV (SEQ ID NO: 1); (b) an HVR-H2 comprising the amino acid sequence of FISSGSSTVYYADTMKG (SEQ ID NO: 2); (c) an HVR-H3 comprising the amino acid sequence of RNYDDWYFDV (SEQ ID NO: 3); (d) an HVR-L1 comprising the amino acid sequence of SASSSVTYMY (SEQ ID NO: 4); (e) an HVR-L2 comprising the amino acid sequence of RTSDLAS (SEQ ID NO: 5); and (f) an HVR-L3 comprising the amino acid sequence of QHYHSYPLT (SEQ ID NO: 6).

In some embodiments, the anti-tryptase antibody (e.g., the anti-tryptase beta antibody) can include (a) a heavy chain variable (VH) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 7; (b) a light chain variable (VL) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 8; or (c) a VH domain as in (a) and a VL domain as in (b). For example, in some embodiments, the VH domain comprises the amino acid sequence of SEQ ID NO: 7. In some embodiments, the VL domain comprises the amino acid sequence of SEQ ID NO: 8. In particular embodiments, the VH domain comprises the amino acid sequence of SEQ ID NO: 7 and the VL domain comprises the amino acid sequence of SEQ ID NO: 8.

In some embodiments, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 9 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some embodiments, the anti-tryptase antibody (e.g., the anti-tryptase beta antibody) includes (a) a heavy chain comprising the amino acid sequence of SEQ ID NO: 9 and (b) a light chain comprising the amino acid sequence of SEQ ID NO: 10.

In other embodiments, the anti-tryptase antibody (e.g., the anti-tryptase beta antibody) can include (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 11 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 10. For example, in some embodiments, the anti-tryptase antibody (e.g., the anti-tryptase beta antibody) includes (a) a heavy chain comprising the amino acid sequence of SEQ ID NO: 11 and (b) a light chain comprising the amino acid sequence of SEQ ID NO: 10.

In still other embodiments, the anti-tryptase antibody (e.g., the anti-tryptase beta antibody) includes at least one, at least two, at least three, at least four, at least five, or all six hypervariable regions (HVRs) selected from (a) an HVR-H1 comprising the amino acid sequence of GYAIT (SEQ ID NO: 12); (b) an HVR-H2 comprising the amino acid sequence of GISSAATTFYSSWAKS (SEQ ID NO: 13); (c) an HVR-H3 comprising the amino acid sequence of DPRGYGAALDRLDL (SEQ ID NO: 14); (d) an HVR-L1 comprising the amino acid sequence of QSIKSVYNNRLG (SEQ ID NO: 15); (e) an HVR-L2 comprising the amino acid sequence of ETSILTS (SEQ ID NO: 16); and (f) an HVR-L3 comprising the amino acid sequence of AGGFDRSGDTT (SEQ ID NO: 17), or a combination of one or more of the above HVRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 12-17. For example, in some embodiments, the anti-tryptase antibody includes (a) an HVR-H1 comprising the amino acid sequence of GYAIT (SEQ ID NO: 12); (b) an HVR-H2 comprising the amino acid sequence of GISSAATTFYSSWAKS (SEQ ID NO: 13); (c) an HVR-H3 comprising the amino acid sequence of DPRGYGAALDRLDL (SEQ ID NO: 14); (d) an HVR-L1 comprising the amino acid sequence of QSIKSVYNNRLG (SEQ ID NO: 15); (e) an HVR-L2 comprising the amino acid sequence of ETSILTS (SEQ ID NO: 16); and (f) an HVR-L3 comprising the amino acid sequence of AGGFDRSGDTT (SEQ ID NO: 17).

In some embodiments, the anti-tryptase antibody (e.g., the anti-tryptase beta antibody) includes (a) a heavy chain variable (VH) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 18; (b) a light chain variable (VL) domain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 19; or (c) a VH domain as in (a) and a VL domain as in (b). For example, in some embodiments, the VH domain comprises the amino acid sequence of SEQ ID NO: 18. In some embodiments, the VL domain comprises the amino acid sequence of SEQ ID NO: 19. In particular embodiments, the VH domain comprises the amino acid sequence of SEQ ID NO: 18 and the VL domain comprises the amino acid sequence of SEQ ID NO: 19.

In some embodiments of any of the preceding methods, the anti-tryptase antibody (e.g., the anti-tryptase beta antibody) includes (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 20 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 21. For example, in some embodiments, the anti-tryptase antibody (e.g., the anti-tryptase beta antibody) includes (a) a heavy chain comprising the amino acid sequence of SEQ ID NO: 20 and (b) a light chain comprising the amino acid sequence of SEQ ID NO: 21.

In other embodiments of any of the preceding methods, the anti-tryptase antibody (e.g., the anti-tryptase beta antibody) includes (a) a heavy chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 22 and (b) a light chain comprising an amino acid sequence having at least 90% sequence identity to (e.g., at least 91%, 92%, 93%, 94%, 95%, 96%, 97%, 98%, or 99% sequence identity), or the sequence of, the amino acid sequence of SEQ ID NO: 21. For example, in some embodiments, the anti-tryptase antibody (e.g., the anti-tryptase beta antibody) includes (a) a heavy chain comprising the amino acid sequence of SEQ ID NO: 22 and (b) a light chain comprising the amino acid sequence of SEQ ID NO: 21.

In some embodiments, the anti-tryptase antibody is an antibody that binds to the same epitope as any one of the preceding antibodies.

Any of the anti-tryptase antibodies disclosed herein can be administered in combination with any of the anti-IgE antibodies described in Subsection C below, including omalizumab (XOLAIR®).

b. IgE B Cell Depleting Antibodies

Any suitable IgE⁺ B cell depleting antibody can be used in the methods of the invention. In some embodiments, the IgE⁺ B cell depleting antibody is an anti-M1′ antibody (e.g., quilizumab). In some embodiments, the anti-M1′ antibody is any anti-M1′ antibody described in International Patent Application Publication No. WO 2008/116149.

c. Anti-IgE Antibodies

Any suitable anti-IgE antibody can be used in the methods of the invention. Exemplary anti-IgE antibodies include rhuMabE25 (E25, omalizumab (XOLAIR®)), E26, E27, as well as CGP-5101 (Hu-901), the HA antibody, ligelizumab, and talizumab. The amino acid sequences of the heavy and light chain variable domains of the humanized anti-IgE antibodies E25, E26 and E27 are disclosed, for example, in U.S. Pat. No. 6,172,213 and WO 99/01556. The CGP-5101 (Hu-901) antibody is described in Corne et al. J. Clin. Invest. 99(5): 879-887, 1997; WO 92/17207; and ATCC Dep. Nos. BRL-10706, BRL-11130, BRL-11131, BRL-11132 and BRL-11133. The HA antibody is described in U.S. Ser. No. 60/444,229, WO 2004/070011, and WO 2004/070010.

For example, in some embodiments, the anti-IgE antibody includes one, two, three, four, five, or all six of the following six HVRs: (a) an HVR-H1 comprising the amino acid sequence of GYSWN (SEQ ID NO: 40); (b) an HVR-H2 comprising the amino acid sequence of SITYDGSTNYNPSVKG (SEQ ID NO: 41); (c) an HVR-H3 comprising the amino acid sequence of GSHYFGHWHFAV (SEQ ID NO: 42); (d) an HVR-L1 comprising the amino acid sequence of RASQSVDYDGDSYMN (SEQ ID NO: 43); (e) an HVR-L2 comprising the amino acid sequence of AASYLES (SEQ ID NO: 44); and (f) an HVR-L3 comprising the amino acid sequence of QQSHEDPYT (SEQ ID NO: 45). In some embodiments, the anti-IgE antibody includes (a) a VH domain comprising an amino acid sequence having at least 90% sequence identity (e.g., at least 91%, 92%, 93%, 94%, 95%, 96%, 97%, 98%, or 99% sequence identity) to the amino acid sequence of SEQ ID NO: 38; (b) a VL domain comprising an amino acid sequence having at least 90% sequence identity (e.g., at least 91%, 92%, 93%, 94%, 95%, 96%, 97%, 98%, or 99% sequence identity) to the amino acid sequence of SEQ ID NO: 39; or (c) a VH domain as in (a) and a VL domain as in (b). In some embodiments, the VH domain comprises the amino acid sequence of SEQ ID NO: 38. In some embodiments, the VL domain comprises the amino acid sequence of SEQ ID NO: 39. In some embodiments, the VH domain comprises the amino acid sequence of SEQ ID NO: 38 and the VL domain comprises the amino acid sequence of SEQ ID NO: 39. Any of the anti-IgE antibodies described herein may be used in combination with any anti-tryptase antibody described in Subsection A above.

1. Antibody Affinity

In certain embodiments, an antibody provided herein (e.g., an anti-tryptase antibody, an anti-FcεR antibody, an IgE⁺ B cell depleting antibody, a mast cell or basophil depleting antibody, an anti-PAR2 antibody, or an anti-IgE antibody) has a dissociation constant (K_{D}) of ≤1 μM, ≤100 nM, ≤10 nM, ≤1 nM, ≤0.1 nM, ≤0.01 nM, ≤1 pM, or ≤0.1 pM (e.g., 10⁻⁶ M or less, e.g., from 10⁻⁶ M to 10⁻⁹M or less, e.g., from 10⁻⁹M to 10⁻¹³ M or less). For example, in some embodiments, an anti-tryptase antibody binds to tryptase (e.g., human tryptase, e.g., human tryptase beta) with a K_{D} of about 100 nM or lower (e.g., 100 nM or lower, 10 nM or lower, 1 nM or lower, 100 pM or lower, 10 pM or lower, 1 pM or lower, or 0.1 pM or lower). In some embodiments, the antibody binds tryptase (e.g., human tryptase, e.g., human tryptase beta) with a K_{D} of 10 nM or lower (e.g., 10 nM or lower, 1 nm or lower, 100 pM or lower, 10 pM or lower, 1 pM or lower, or 0.1 pM or lower). In some embodiments, the antibody binds tryptase (e.g., human tryptase, e.g., human tryptase beta) with a K_{D} of 1 nM or lower (e.g., 1 nm or lower, 100 pM or lower, 10 pM or lower, 1 pM or lower, or 0.1 pM or lower). In some embodiments, any of the anti-tryptase antibodies described above or herein binds to tryptase (e.g., human tryptase, e.g., human tryptase beta) with a K_{D} of about 0.5 nM or lower (e.g., 0.5 nm or lower, 400 pM or lower, 300 pM or lower, 200 pM or lower, 100 pM or lower, 50 pM or lower, 25 pM or lower, 10 pM or lower, 1 pM or lower, or 0.1 pM or lower). In some embodiments, the antibody binds tryptase (e.g., human tryptase, e.g., human tryptase beta) with a K_{D} between about 0.1 nM to about 0.5 nM (e.g., about 0.1 nM, about 0.2 nM, about 0.3 nM, about 0.4 nM, or about 0.5 nM). In some embodiments, the antibody binds tryptase (e.g., human tryptase, e.g., human tryptase beta) with a K_{D} of about 0.4 nM. In some embodiments, the antibody binds tryptase (e.g., human tryptase, e.g., human tryptase beta) with a K_{D} of about 0.18 nM. Any of the other antibodies described herein may bind to its antigen with affinities as described above with respect to anti-tryptase antibodies.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al. J. Mol. Biol. 293:865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al. Cancer Res. 57:4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN®-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′ (3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in phosphate buffered saline (PBS) with 0.05% polysorbate 20 (TWEEN®-20) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al. (J. Mol. Biol. 293:865-881, 1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some embodiments, K_{D} is measured using a BIACORE® SPR assay. In some embodiments, the SPR assay can use a BIAcore® T200 or an equivalent device. In some embodiments, BIAcore® Series S CM5 sensor chips (or equivalent sensor chips) are immobilized with monoclonal mouse anti-human IgG (Fc) antibody and anti-tryptase antibodies are subsequently captured on the flow cell. Serial 3-fold dilutions of the His-tagged human tryptase beta 1 monomer (SEQ ID NO: 128) are injected at a flow rate of 30 μl/min. Each sample is analyzed with 3 min association and 10 min dissociation. The assay is performed at 25° C. After each injection, the chip is regenerated using 3 M MgCl₂. Binding response is corrected by subtracting the response units (RU) from a flow cell capturing an irrelevant IgG at similar density. A 1:1 Languir model of simultaneous fitting of kₒₙ and k_{off} is used for kinetics analysis.

2. Antibody Fragments

In certain embodiments, an antibody provided herein (e.g., an anti-tryptase antibody, an anti-FcεR antibody, an IgE⁺ B cell depleting antibody, a mast cell or basophil depleting antibody, an anti-PAR2 antibody, or an anti-IgE antibody) is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134, 2003; and Hollinger et al. Proc. Natl. Acad. Sci. USA 90: 6444-6448, 1993. Triabodies and tetrabodies are also described in Hudson et al. Nat. Med. 9:129-134, 2003.

Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain embodiments, an antibody provided herein (e.g., an anti-tryptase antibody, an anti-FcεR antibody, an IgE⁺ B cell depleting antibody, a mast cell or basophil depleting antibody, an anti-PAR2 antibody, or an anti-IgE antibody) is a chimeric antibody. Certain chimeric antibodies are described, e.g., in U.S. Pat. No. 4,816,567; and Morrison et al. Proc. Natl. Acad. Sci. USA, 81:6851-6855, 1984). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:1619-1633, 2008, and are further described, e.g., in Riechmann et al. Nature 332:323-329, 1988; Queen et al. Proc. Natl. Acad. Sci. USA 86:10029-10033, 1989; U.S. Pat. Nos. 5,821,337, 7,527,791, 6,982,321, and 7,087,409; Kashmiri et al. Methods 36:25-34, 2005 (describing specificity determining region (SDR) grafting); Padlan, Mol. Immunol. 28:489-498, 1991 (describing “resurfacing”); Dall'Acqua et al. Methods 36:43-60, 2005 (describing “FR shuffling”); and Osbourn et al. Methods 36:61-68, 2005 and Klimka et al. Br. J. Cancer, 83:252-260, 2000 (describing the “guided selection” approach to FR shuffling).

Human framework regions that may be used for humanization include but are not limited to: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al. Proc. Natl. Acad. Sci. USA, 89:4285, 1992; and Presta et al. J. Immunol., 151:2623, 1993); human mature (somatically mutated) framework regions or human germline framework regions (see, e.g., Almagro et al. Front. Biosci. 13:1619-1633, 2008); and framework regions derived from screening FR libraries (see, e.g., Baca et al. J. Biol. Chem. 272:10678-10684, 1997 and Rosok et al. J. Biol. Chem. 271:22611-22618, 1996).

4. Human Antibodies

In certain embodiments, an antibody provided herein (e.g., an anti-tryptase antibody, an anti-FcεR antibody, an IgE⁺ B cell depleting antibody, a mast cell or basophil depleting antibody, an anti-PAR2 antibody, or an anti-IgE antibody) is a human antibody. Human antibodies can be produced using various techniques known in the art. Human antibodies are described generally in van Dijk et al. Curr. Opin. Pharmacol. 5:368-74, 2001 and Lonberg, Curr. Opin. Immunol. 20:450-459, 2008.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125, 2005. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001, 1984; Brodeur et al. Monoclonal Antibody Production Techniques and Applications, pp. 51-63 (Marcel Dekker, Inc., New York, 1987); and Boerner et al. J. Immunol. 147: 86, 1991). Human antibodies generated via human B-cell hybridoma technology are also described in Li et al. Proc. Natl. Acad. Sci. USA, 103:3557-3562, 2006. Additional methods include those described, for example, in U.S. Pat. No. 7,189,826 (describing production of monoclonal human IgM antibodies from hybridoma cell lines) and Ni, Xiandai Mianyixue, 26(4):265-268, 2006 (describing human-human hybridomas). Human hybridoma technology (Trioma technology) is also described in Vollmers et al. Histology and Histopathology 20(3):927-937, 2005 and Vollmers et al. Methods and Findings in Experimental and Clinical Pharmacology 27(3):185-91, 2005.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al. Nature 348:552-554, 1990; Clackson et al. Nature 352: 624-628, 1991; Marks et al. J. Mol. Biol. 222: 581-597, 1992; Marks et al. in Methods in Molecular Biology 248:161-175 (Lo, ed., Human Press, Totowa, N.J., 2003); Sidhu et al. J. Mol. Biol. 338(2): 299-310, 2004; Lee et al. J. Mol. Biol. 340(5): 1073-1093, 2004; Fellouse, Proc. Natl. Acad. Sci. USA 101(34):12467-12472, 2004; and Lee et al. J. Immunol. Methods 284(1-2): 119-132, 2004.

In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol., 12: 433-455, 1994. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. EMBO J. 12: 725-734, 1993. Finally, naive libraries can also be made synthetically by cloning unrearranged V-gene segments from stem cells, and using PCR primers containing random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein (e.g., an anti-tryptase antibody, an anti-FcεR antibody, an IgE⁺ B cell depleting antibody, a mast cell or basophil depleting antibody, an anti-PAR2 antibody, or an anti-IgE antibody) is a multispecific antibody, for example, a bispecific antibody. Multispecific antibodies are monoclonal antibodies that have binding specificities for at least two different sites. For example, with respect to anti-tryptase antibodies, in certain embodiments, bispecific antibodies may bind to two different epitopes of tryptase. In certain embodiments, one of the binding specificities is for tryptase and the other is for any other antigen (e.g., a second biological molecule). In some embodiments, bispecific antibodies may bind to two different epitopes of tryptase. In other embodiments, one of the binding specificities is for tryptase (e.g., human tryptase, e.g., human tryptase beta) and the other is for any other antigen (e.g., a second biological molecule, e.g., IL-13, IL-4, IL-5, IL-17, IL-33, IgE, M1 prime, CRTH2, or TRPA). Accordingly, the bispecific antibody may have binding specificity for tryptase and IL-13; tryptase and IgE; tryptase and IL-4; tryptase and IL-5; tryptase and IL-17, or tryptase and IL-33. In particular, the bispecific antibody may have binding specificity for tryptase and IL-13 or tryptase and IL-33. In other particular embodiments, the bispecific antibody may have binding specificity for tryptase and IgE. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein et al. Nature 305: 537, 1983; WO 93/08829; and Traunecker et al. EMBO J. 10: 3655, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al. Science, 229: 81, 1985); using leucine zippers to produce bispecific antibodies (see, e.g., Kostelny et al. J. Immunol., 148(5):1547-1553, 1992); using “diabody” technology for making bispecific antibody fragments (see, e.g., Hollinger et al. Proc. Natl. Acad. Sci. USA 90:6444-6448, 1993); and using single-chain Fv (scFv) dimers (see, e.g., Gruber et al. J. Immunol. 152:5368, 1994); and preparing trispecific antibodies as described, e.g., in Tutt et al. J. Immunol. 147: 60, 1991.

Engineered antibodies with three or more functional antigen binding sites, including “Octopus antibodies,” are also included herein (see, e.g., US 2006/0025576A1).

The antibody or fragment herein also includes a “Dual Acting Fab” or “DAF” comprising an antigen binding site that binds to tryptase as well as another, different antigen (see, US 2008/0069820, for example).

### Knobs-into-Holes

The use of knobs-into-holes as a method of producing multispecific antibodies is described, e.g., in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin. 26:1-9. A brief nonlimiting discussion is provided below.

A “protuberance” refers to at least one amino acid side chain which projects from the interface of a first polypeptide and is therefore positionable in a compensatory cavity in the adjacent interface (i.e., the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some embodiments, a nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US 2011/0287009 or Table 1 of U.S. Pat. No. 7,642,228.

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiments, the original residue for the formation of the protuberance has a small side chain volume, such as alanine, asparagine, aspartic acid, glycine, serine, threonine, or valine. See, for example, U.S. Pat. No. 7,642,228.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g., by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T), and valine (V). In some embodiments, an import residue is serine, alanine, or threonine. In some embodiments, the original residue for the formation of the cavity has a large side chain volume, such as tyrosine, arginine, phenylalanine, or tryptophan.

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe, and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely-accepted techniques in the art.

In some embodiments, a knob mutation in an IgG1 constant region is T366W. In some embodiments, a hole mutation in an IgG1 constant region comprises one or more mutations selected from T366S, L368A, and Y407V. In some embodiments, a hole mutation in an IgG1 constant region comprises T366S, L368A, and Y407V.

In some embodiments, a knob mutation in an IgG4 constant region is T366W. In some embodiments, a hole mutation in an IgG4 constant region comprises one or more mutations selected from T366S, L368A, and Y407V. In some embodiments, a hole mutation in an IgG4 constant region comprises T366S, L368A, and Y407V.

7. Antibody Variants

In certain embodiments, amino acid sequence variants of the antibodies provided herein are contemplated. For example, it may be desirable to improve the binding affinity and/or other biological properties of the antibody, such as inhibitory activity. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196, 2008), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al. ed., Human Press, Totowa, N.J., 2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham et al. Science 244:1081-1085, 1989. In this method, a residue or group of target residues (e.g., charged residues such as Arg, Asp, His, Lys, and Glu) are identified and replaced by a neutral or negatively charged amino acid (e.g., Ala or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32, 1997. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. 2003/0157108 and 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249, 2004; Yamane-Ohnuki et al. Biotech. Bioeng. 87: 614, 2004. Examples of cell lines capable of producing defucosylated antibodies include Lec13 CHO cells deficient in protein fucosylation (Ripka et al. Arch. Biochem. Biophys. 249:533-545, 1986; US 2003/0157108; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87: 614, 2004; Kanda et al. Biotechnol. Bioeng. 94(4):680-688, 2006; and WO 2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3, or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch et al. Annu. Rev. Immunol. 9:457-492, 1991. Non-limiting examples of in vitro assays to assess ADCC activity of a molecule of interest is described in U.S. Pat. No. 5,500,362 (see, e.g., Hellstrom et al. Proc. Natl. Acad. Sci. USA 83:7059-7063, 1986 and Hellstrom et al. Proc. Natl. Acad. Sci. USA 82:1499-1502, 1985; U.S. Pat. No. 5,821,337 (see Bruggemann et al. J. Exp. Med. 166:1351-1361, 1987). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al. Proc. Natl. Acad. Sci. USA 95:652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al. J. Immunol. Methods 202:163, 1996; Cragg et al. Blood 101:1045-1052, 2003; and Cragg et al. Blood 103:2738-2743, 2004). FcRn binding and in vivo clearance/half life determinations can also be performed using methods known in the art (see, e.g., Petkova et al. Intl. Immunol. 18(12):1759-1769, 2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312; and Shields et al. J. Biol. Chem. 9(2): 6591-6604, 2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), for example, as described in U.S. Pat. No. 6,194,551, WO 99/51642, and Idusogie et al. J. Immunol. 164: 4178-4184, 2000.

Antibodies with increased half lives and improved binding to the neonatal Fc receptor (FcRn), which is responsible for the transfer of maternal IgGs to the fetus (Guyer et al. J. Immunol. 117:587, 1976 and Kim et al. J. Immunol. 24:249, 1994), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan et al. Nature 322:738-40, 1988; U.S. Pat. Nos. 5,648,260 and 5,624,821; and WO 94/29351 concerning other examples of Fc region variants.

d) Cysteine Engineered Antibody Variants

In certain embodiments, it may be desirable to create cysteine engineered antibodies, for example, “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, and the like.

In another embodiment, conjugates of an antibody and nonproteinaceous moiety that may be selectively heated by exposure to radiation are provided. In one embodiment, the nonproteinaceous moiety is a carbon nanotube (Kam et al. Proc. Natl. Acad. Sci. USA 102: 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Pharmaceutical Formulations

Therapeutic formulations including therapeutic agents used in accordance with the present invention (e.g., any of the tryptase antagonists (e.g., anti-tryptase antibodies, including any of the anti-tryptase antibodies described herein), FcεR antagonists, IgE⁺ B cell depleting antibodies, mast cell or basophil depleting antibodies, PAR2 antagonists, IgE antagonists (e.g., anti-IgE antibodies, e.g., omalizumab (XOLAIR®)), and combinations thereof (e.g., a tryptase antagonist (e.g., an anti-tryptase antibody, including any of the anti-tryptase antibodies described herein) and an IgE antagonist (e.g., an anti-IgE antibody, e.g., omalizumab (XOLAIR®))), and/or additional therapeutic agents described herein) are prepared for storage by mixing the therapeutic agent(s) having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol. 119, Marcel Dekker, 2002.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound, preferably those with complementary activities that do not adversely affect each other. The type and effective amounts of such medicaments depend, for example, on the amount and type of the therapeutic agent(s) present in the formulation, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### EXAMPLES

The following examples are provided to illustrate, but not to limit the presently claimed invention.

### Example 1: Materials and Methods

A) Active Tryptase Allele Count

PCR followed by Sanger sequencing of genomic DNA was employed to determine active tryptase allele count as described previously (Trivedi et al. J. Allergy Clin. Immunol. 124:1099-1105 e1-4, 2009). In brief, active tryptase allele count was assessed as the number of remaining active tryptase genes after accounting for tryptase deficiency alleles, i.e., those determining α and βIII^{FS}. Genotypes were automatically called using the intensity ratio of the two (A/B) alleles. Patients were assigned to genotype bin based on this ratio. Genotypes were confirmed by visual inspection of the sequencing traces for 5% of the population without error. Patient data that did not bin properly were visually inspected. Genotyping for active tryptase allele count was conducted on European ancestry asthma subjects determined by principal components analysis of genome wide SNP data as described previously (Ramirez-Carrozzi et al. J. Allergy Clin. Immunol. 135:1080-1083 e3, 2015).

To genotype tryptase α, the forward primer 5′-CTG GTG TGC AAG GTG AAT GG-3′ (SEQ ID NO: 31) and the reverse primer 5′-AGG TCC AGO ACT CAG GAG GA-3′ (SEQ ID NO: 32) were used to amplify a portion of the TPSAB1 locus. The PCR conditions were as follows: Qiagen HOTSTARTAQ® Plus polymerase was used during the thermocycler conditions of 95° C. for 5 min, followed by 35 cycles of 94° C. for 60 seconds, 58° C. for 60 seconds, and 72° C. for 2 min. Following PCR, EXOSAP-IT™ PCR product cleanup reagent was used for cleanup. The same forward and reverse primers were used for sequencing. Sequencing was performed using BIG-DYE® terminator chemistry on an ABI 3730XL DNA analyzer manufactured by Applied Biosystems.

To genotype tryptase βIII^{FS}, the forward primer 5′-GCA GGT GAG OCT GAG AGT CC-3′ (SEQ ID NO: 33) and the reverse primer 5′-GGG ACC TTC ACC TOO TTC AG-3′ (SEQ ID NO: 34) were used to amplify a portion of the TPSB2 locus. The PCR conditions were as follows: Qiagen HOTSTARTAQ® Plus polymerase was used during the thermocycler conditions of 95° C. for 5 min, followed by 35 cycles of 94° C. for 60 seconds, 60° C. for 60 seconds, and 72° C. for 2 min. Following PCR, EXOSAP-IT™ PCR product cleanup reagent was used for cleanup. For sequencing, the forward primer 5′-GCA GGT GAG OCT GAG AGT CC-3′ (SEQ ID NO: 33) and the reverse sequencing primer 5′-CAG CCA GTG ACC CAG CAC-3′ (SEQ ID NO: 35) were used. Sequencing was performed using BIG-DYE® terminator chemistry on an ABI 3730XL DNA analyzer manufactured by Applied Biosystems.

B) Clinical Cohorts

EXTRA (ClinicalTrials.gov identifier: NCT00314574) was a randomized, double-blind, placebo-controlled study of Xolair (anti-IgE) in subjects 12-75 years old with moderate to severe persistent asthma. Full details of the study design have been published previously (Hanania et al. Ann. Intern. Med. 154:573-582, 2011; Hanania et al. Am. J. Respir. Crit. Care Med. 187:804-811, 2013; Choy et al. J. Allergy Clin. Immunol. 138:1230-1233 e8, 2016). In brief, after a 2- to 4-week run-in period, eligible patients were randomized in a 1:1 ratio to receive XOLAIR® (omalizumab) or placebo (in addition to high-dose inhaled corticosteroids (ICS) and long-acting beta-adrenoceptor agonists (LABA), with or without additional controller medications) for 48 weeks.

BOBCAT (Arron et al. Eur. Respir. J. 43:627-629, 2014; Choy et al. supra; Huang et al. J. Allergy Clin. Immunol. 136:874-884, 2015; Jia et al. J. Allergy Clin. Immunol. 130:647-654, 2012) was a multicenter observational study conducted in the United States, Canada, and the United Kingdom of 67 adult patients with moderate-to-severe asthma. Inclusion criteria required a diagnosis of moderate-to-severe asthma (confirmed by a forced expiratory volume in 1 second (FEV₁) between 40% and 80% of predicted value, as well as evidence within the past 5 years of >12% reversibility of airway obstruction with a short-acting bronchodilator or methacholine sensitivity (provocation concentration causing a 20% fall in FEV₁ (PC20) of <8 mg/mL) that was uncontrolled (as defined by at least 2 exacerbations in the prior year or a score of >1.50 on the Asthma Control Questionnaire (ACQ) 5-item version (ACQ-5) while receiving a stable dose regimen (>6 weeks) of a high-dose ICS (>1000 mg fluticasone or equivalent per day)) with or without a LABA.

MILLY (ClinicalTrials.gov identifier: NCT00930163) was a randomized, double-blind, placebo controlled study of lebrikizumab (anti-IL-13 antibody) in adults who had asthma that was inadequately controlled despite inhaled glucocorticoid therapy (Corren et al. N. Engl. J. Med. 365:1088-1098, 2011).

C) Total Tryptase ELISA

Serum or plasma tryptase levels were measured using a sandwich enzyme-linked immunosorbent assay (ELISA) with 2 monoclonal antibodies capable of detecting monomers and tetramers of the human tryptases β1, β2, β3, and α1. Briefly, 384-well plates were coated with monoclonal anti-tryptase antibody at 1.0 μg/ml in phosphate-buffered saline (PBS) buffer overnight at 4° C. and were then blocked with 90 μl of blocking buffer (1×PBS+1% bovine serum albumin (BSA)) for at least 1 h at room temperature. Serum or plasma samples were diluted 1:100 in assay buffer (1×PBS pH 7.4, 0.35 M NaCl, 0.5% BSA, 0.05% TWEEN® 20 (polysorbate 20), 0.25% 3-[(3-cholamidopropyl)dimethylammonio]-1-propanesulfonate (CHAPS), 5 mM ethylenediaminetetraacetic acid (EDTA), and 15 parts per million (PPM) PROCLIN™ (broad spectrum antimicrobial)) and added in triplicates to the plates after washing, and incubated with agitation at room temperature for 2 h at room temperature. Recombinant tryptase β1 was used to establish a standard range (7.8-500 pg/ml) in the assay. After washing, biotinylated anti-human tryptase (0.5 μg/ml) in assay diluent (1×PBS pH 7.4, 0.5% BSA, 0.05% TWEEN® 20) were added and incubated for 1 h at room temperature. Color was developed after washing with streptavidin-peroxidase and substrate tetramethylbenzidine (TMB). The data were interpreted based on a 4-parameter (4P)-fit standard curve. The detection limit of this assay was approximately 7.8 pg/ml.

D) Statistics

R software (RCoreTeam, R: A Language an Environment for Statistical Computing, 2014) was used for plotting and analysis.

### Example 2: Active Tryptase Gene Count is Heterogeneous in Moderate to Severe Asthma

Tryptase is a granule protein that is significantly expressed in mast cells and has been implicated as an important asthma mediator, having notable effects on lung function. The genes encoding enzymatically active tryptase, TSPAB1 and TPSB2, are polymorphic, and we have previously described the frequencies and pattern of inheritance of common, inactivating, loss of function mutations (Trivedi et al. J. Allergy Clin. Immunol. 124:1099-1105 e1-4, 2009). Despite the advent of modern whole genome analyses, including high density SNP arrays and next generation sequencing, tryptase loci have not been well studied because the high homology and repetitive nature of this region is not amenable for these methodologies, thus requiring direct re-sequencing. We hypothesized that active tryptase allele count, inferred by accounting for inactivating mutations of TSPAB1 and TPSB2, would affect the expression of mast cell-derived tryptase and predict clinical response to mast cell-related therapies, e.g., XOLAIR® (omalizumab), an anti-IgE antibody.

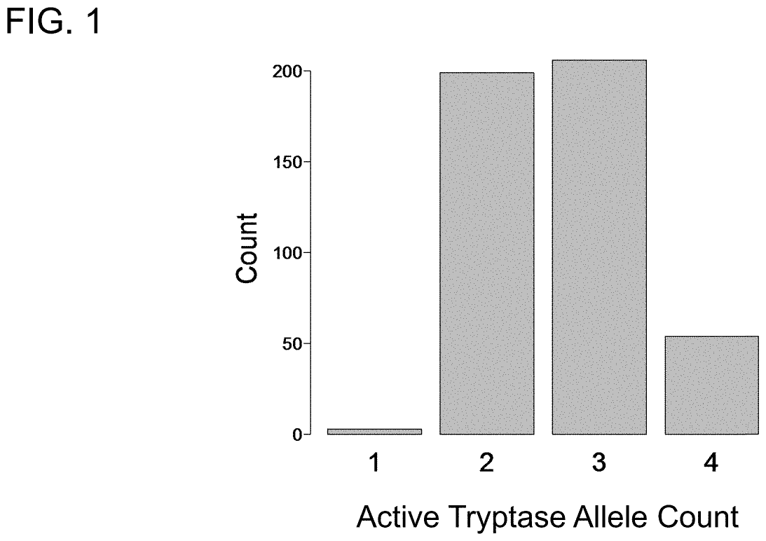

We assessed active tryptase allele count in moderate to severe asthma subjects of European ancestry from the BOBCAT, EXTRA, and MILLY studies (see Example 1). Consistent with previous reports in world populations (Trivedi et al. J. Allergy Clin. Immunol. 124:1099-1105 e1-4, 2009), loss of function mutations were common in subjects in our study (FIG. 1); 88.3% of the subjects (408 of 462) had at least one loss of function mutation, yielding 1, 2, or 3 remaining active tryptase copies. Subjects having zero active copies were not observed in these studies, and those having one active copy was relatively rare (<1%, 3 of 462). Subjects having two or three active tryptase copies predominated in our cohort (88%, 405 of 462); prevalence for two or three active copies were comparable (43%, 199 or 462; and 45%, 206 of 462 respectively).

The observed distribution of active tryptase allele count is consistent with the finding that specific alleles of TPSAB1 and TPSB2 are in linkage disequilibrium, leading to dysfunctional tryptase alleles being co-inherited with functional alleles (Trivedi et al. supra). Thus, subjects with zero or four active tryptase allele counts are expected to be rare. In summary, active tryptase allele count is heterogeneous in moderate to severe asthma patients.

### Example 3: Active Tryptase Allele Count is a Protein Quantitative Trait Linkage (pQTL) for Asthmatic Peripheral Tryptase Levels

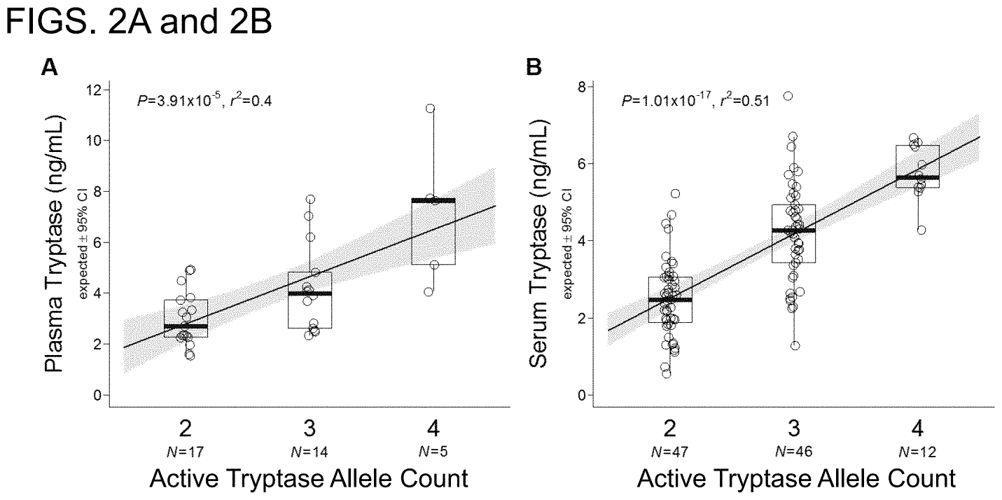

Next, we assessed the relationship of active tryptase copy number with total peripheral tryptase levels in moderate to severe asthma from BOBCAT (FIG. 2A) and MILLY (FIG. 2B) studies. A significant pQTL (P<0.0001) was observed in each study, further linking that active tryptase allele count is an underlying determinant of tryptase expression and that asthma subjects with increased active tryptase allele counts are associated with elevated tryptase expression levels. In summary, these data demonstrate that the expression level of peripheral tryptase (e.g., in blood samples) are correlated with the subject's active tryptase allele count. Based on this correlation, it is expected that the expression level of tryptase, for example, in blood (e.g., serum or plasma) can be used to predict treatment response, for example, to anti-IgE therapy or other therapeutic interventions.

### Example 4: Active Tryptase Allele Count Predicts Asthmatic FEV₁ Response to Anti-IgE Therapy

Based on the findings that active tryptase allele count is correlated with the expression of active tryptase from primary mast cells ex vivo and with peripheral levels of total tryptase in asthma patients (see Example 3), we hypothesized that active tryptase allele count would predict clinical response to a mast cell-related therapy in asthma. XOLAIR® (omalizumab) is an approved anti-IgE monoclonal antibody therapy for the reduction of asthma exacerbations for atopic asthma. As blocking IgE leads to the amelioration of clinical asthma by reducing IgE/FcεRI-dependent degranulation from mast cells, we conducted a post-hoc analysis of FEV₁ improvement from baseline on the basis of active tryptase allele count. As two and three active tryptase alleles were predominantly (88%) observed in asthma, and therefore subjects with one or four active tryptase alleles are relatively rare, we dichotomized our study population as having 1 or 2 versus 3 or 4 active tryptase alleles to improve statistical power.

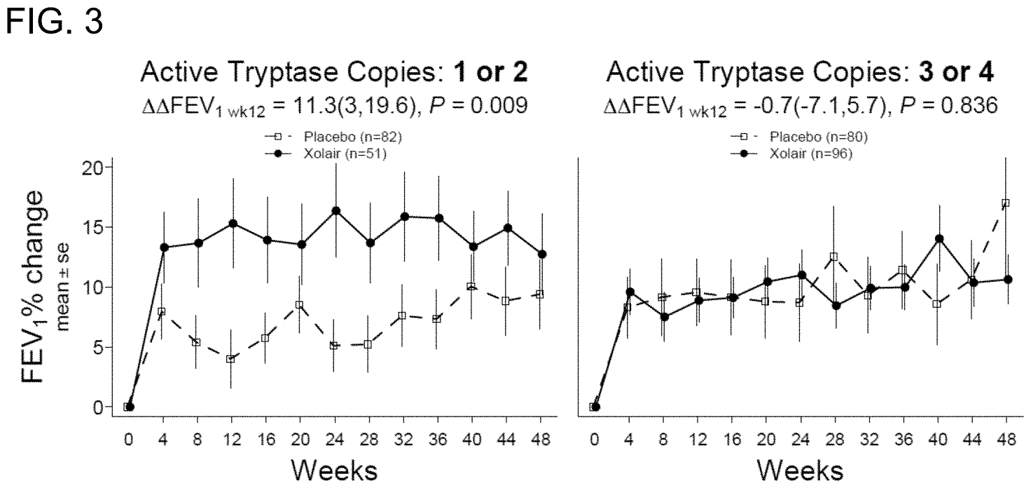

Subjects having one or two active tryptase alleles derived a significant FEV₁ percent improvement by week 12 with anti-IgE therapy (FIG. 3, mean±standard error=11.3(3, 19.6)%, P=0.009). In contrast, subjects with three or four active tryptase alleles did not derive FEV₁ benefit from anti-IgE therapy (FIG. 3). These observations were sustained throughout the 48 weeks of the study. Therefore, asthmatic subjects with one or two tryptase active alleles derived significant lung function improvements to anti-IgE (XOLAIR®) therapy as compared to subjects having three or four copy numbers.

Mast cell tryptase has been shown to directly affect airway smooth muscles by increasing contractility and cell differentiation in vitro, and therefore has been implicated as an important asthma mediator of airway obstruction. These data suggest that anti-IgE therapy may be most effective in subjects who express low levels of mast cell tryptase which may be released by both IgE/FcεRI-dependent degranulation as well as IgE/FcεRI-independent mechanisms. These data also indicate that active tryptase allele count can be used as a predictive biomarker for predicting response to asthma therapeutic interventions. For example, patients with low active tryptase allele count are likely to benefit from therapy with XOLAIR® (omalizumab). In other examples, patients with high active tryptase allele count are likely to benefit from therapy with tryptase antagonists (e.g., anti-tryptase antibodies).

### Example 5: Active Tryptase Allele Count does not Associate with Type 2 Biomarkers in Moderate to Severe Asthma

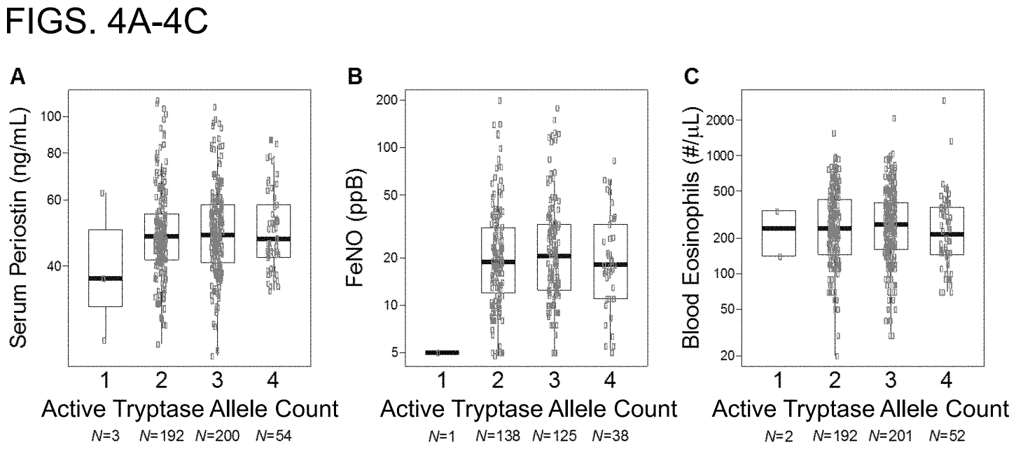

Previous studies showed that the expression levels of Type 2 biomarkers enriched for treatment benefit, i.e., exacerbation rate reduction, to XOLAIR® (omalizumab) therapy in asthma (Hanania et al. Am. J. Respir. Crit. Care Med. 187:804-811, 2013). To investigate how active tryptase allele count relates to biomarkers of Type 2 inflammation, we assessed the levels of serum periostin, fractional exhaled nitric oxide (FeNO), and blood eosinophil counts with respect to active tryptase allele count from subjects at baseline from BOBCAT, EXTRA, and MILLY studies and did not observe any relationship (FIGS. 4A-4C). These data indicate that active tryptase allele count and Type 2 biomarkers independently select different subsets of asthmatics. The independence of active tryptase copy number with respect to levels of biomarkers of Type 2 inflammation suggest that active tryptase copy number assessment provides unique information to tryptase and mast cell biology. For example, subjects who have increased active tryptase allele counts and low Type 2 biomarker levels (e.g., T_{H}2-low asthma) may benefit from treatment with a mast cell-directed therapy (e.g., a therapy including a tryptase antagonist, an IgE⁺ B cell depleting antibody, a mast cell or basophil depleting antibody, or a protease activated receptor 2 (PAR2) antagonist). Conversely, subjects with increased active tryptase allele counts and high Type 2 biomarker levels (e.g., T_{H}2-high asthma) may benefit from treatment with a T_{H}2 pathway inhibitor and/or a mast cell-directed therapy.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating a patient having a mast cell-mediated inflammatory disease who has been identified as having (i) a genotype comprising an active tryptase allele count that is at or above a reference active tryptase allele count; or (ii) an expression level of tryptase in a sample from the patient that is at or above a reference level of tryptase, the method comprising administering to a patient having a mast cell-mediated inflammatory disease a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof.

2. A method of determining whether a patient having a mast cell-mediated inflammatory disease is likely to respond to a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof, the method comprising:
(a) determining in a sample from a patient having a mast cell-mediated inflammatory disease the patient's active tryptase allele count; and
(b) identifying the patient as likely to respond to a therapy comprising an agent selected from the group consisting of a tryptase antagonist, an IgE⁺ B cell depleting antibody, a mast cell or basophil depleting antibody, a PAR2 antagonist, and a combination thereof based on the patient's active tryptase allele count, wherein an active tryptase allele count at or above a reference active tryptase allele count indicates that the patient has an increased likelihood of being responsive to the therapy.

3. A method of determining whether a patient having a mast cell-mediated inflammatory disease is likely to respond to a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof, the method comprising:
(a) determining the expression level of tryptase in a sample from a patient having a mast cell-mediated inflammatory disease; and
(b) identifying the patient as likely to respond to a therapy comprising an agent selected from the group consisting of a tryptase antagonist, an IgE⁺ B cell depleting antibody, a mast cell or basophil depleting antibody, a PAR2 antagonist, and a combination thereof based on the expression level of tryptase in the sample from the patent, wherein an expression level of tryptase in the sample at or above a reference level of tryptase indicates that the patient has an increased likelihood of being responsive to the therapy.

4. The method of claim 2 or 3, further comprising administering the therapy to the patient.

5. The method of any one of claims 1-4, wherein the patient has been identified as having a level of a Type 2 biomarker in a sample from the patient that is below a reference level of the Type 2 biomarker.

6. The method of claim 5, wherein the agent is administered to the patient as a monotherapy.

7. The method of any one of claims 1-4, wherein the patient has been identified as having a level of a Type 2 biomarker in a sample from the patient that is at or above a reference level of the Type 2 biomarker.

8. The method of claim 7, wherein the method further comprises administering an additional T_{H}2 pathway inhibitor to the patient.

9. A method of treating a patient having a mast cell-mediated inflammatory disease who has been identified as having (i) a genotype comprising an active tryptase allele count that is below a reference active tryptase allele count; or (ii) an expression level of tryptase in a sample from the patient that is below a reference level of tryptase, the method comprising administering to a patient having a mast cell-mediated inflammatory disease a therapy comprising an IgE antagonist or an Fc epsilon receptor (FcεR) antagonist.

10. A method of determining whether a patient having a mast cell-mediated inflammatory disease is likely to respond to a therapy comprising an IgE antagonist or an FcεR antagonist, the method comprising:
(a) determining in a sample from a patient having a mast cell-mediated inflammatory disease the patient's active tryptase allele count; and
(b) identifying the patient as likely to respond to a therapy comprising an IgE antagonist or an FcεR antagonist based on the patient's active tryptase allele count, wherein an active tryptase allele count below a reference active tryptase allele count indicates that the patient has an increased likelihood of being responsive to the therapy.

11. A method of determining whether a patient having a mast cell-mediated inflammatory disease is likely to respond to a therapy comprising an IgE antagonist or an FcεR antagonist, the method comprising:
(a) determining the expression level of tryptase in a sample from a patient having a mast cell-mediated inflammatory disease; and
(b) identifying the patient as likely to respond to a therapy comprising an IgE antagonist or an FcεR antagonist based on the expression level of tryptase in the sample from the patient, wherein an expression level of tryptase in the sample from the patient below a reference level of tryptase indicates that the patient has an increased likelihood of being responsive to the therapy.

12. The method of claim 10 or 11, further comprising administering the therapy to the patient.

13. The method of any one of claims 10-12, wherein the patient has been identified as having a level of a Type 2 biomarker in a sample from the patient that is at or above a reference level of the Type 2 biomarker.

14. The method of claim 13, wherein the method further comprises administering an additional T_{H}2 pathway inhibitor to the patient.

15. A method of selecting a therapy for a patient having a mast cell-mediated inflammatory disease, the method comprising:
(a) determining in a sample from a patient having a mast cell-mediated inflammatory disease the patient's active tryptase allele count; and
(b) selecting for the patient:
(i) a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof if the patient's active tryptase allele count is at or above a reference active tryptase allele count, or
(ii) a therapy comprising an IgE antagonist or an FcεR antagonist if the patient's active tryptase allele count is below a reference active tryptase allele count.

16. A method of selecting a therapy for a patient having a mast cell-mediated inflammatory disease, the method comprising:
(a) determining the expression level of tryptase in a sample from a patient having a mast cell-mediated inflammatory disease; and
(b) selecting for the patient:
(i) a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof if the expression level of tryptase in the sample from the patient is at or above a reference level of tryptase, or
(ii) a therapy comprising an IgE antagonist or an FcεR antagonist if the expression level of tryptase in the sample from the patient is below a reference level of tryptase.

17. The method of claim 15 or 16, further comprising administering the therapy selected in accordance with (b) to the patient.

18. The method of any one of claims 15-17, wherein the patient has been identified as having a level of a Type 2 biomarker in a sample from the patient that is below a reference level of the Type 2 biomarker.

19. The method of claim 18, wherein the agent is administered to the patient as a monotherapy.

20. The method of any one of claims 15-17, wherein the patient has been identified as having a level of a Type 2 biomarker in a sample from the patient that is at or above a reference level of the Type 2 biomarker, and the method further comprises selecting a combination therapy that comprises a T_{H}2 pathway inhibitor.

21. The method of claim 20, wherein the method further comprises administering a T_{H}2 pathway inhibitor to the patient.

22. A method for assessing a response of a patient having a mast cell-mediated inflammatory disease to treatment with a therapy comprising an agent selected from the group consisting of a tryptase antagonist, an IgE⁺ B cell depleting antibody, a mast cell or basophil depleting antibody, a protease activated receptor 2 (PAR2) antagonist, and a combination thereof, the method comprising:
(a) determining the expression level of tryptase in a sample from a patient having a mast cell-mediated inflammatory disease at a time point during or after administration of a therapy comprising an agent selected from the group consisting of a tryptase antagonist, an IgE⁺ B cell depleting antibody, a mast cell or basophil depleting antibody, a PAR2 antagonist, and a combination thereof to the patient; and
(b) maintaining, adjusting, or stopping the treatment based on a comparison of the expression level of tryptase in the sample with a reference level of tryptase,
wherein a change in the expression level of tryptase in the sample from the patient compared to the reference level is indicative of a response to treatment with the therapy.

23. The method of claim 22, wherein the change is an increase in the expression level of tryptase and the treatment is maintained.

24. The method of claim 23, wherein the change is a decrease in the expression level of tryptase and the treatment is adjusted or stopped.

25. A method for monitoring the response of a patient having a mast cell-mediated inflammatory disease treated with a therapy comprising an agent selected from the group consisting of a tryptase antagonist, an IgE⁺ B cell depleting antibody, a mast cell or basophil depleting antibody, a protease activated receptor 2 (PAR2)antagonist, and a combination thereof, the method comprising:
(a) determining the expression level of tryptase in a sample from the patient at a time point during or after administration of the therapy comprising an agent selected from the group consisting of a tryptase antagonist, an IgE⁺ B cell depleting antibody, a mast cell or basophil depleting antibody, a PAR2 antagonist, and a combination thereof to the patient; and
(b) comparing the expression level of tryptase in the sample from the patient with a reference level of tryptase, thereby monitoring the response of the patient undergoing treatment with the therapy.

26. The method of claim 25, wherein the change is an increase in the level of tryptase and the treatment is maintained.

27. The method of claim 25, wherein the change is a decrease in the expression level of tryptase and the treatment is adjusted or stopped.

28. The method of any one of claim 1, 2, 4-10, 12-15, or 17-21, wherein the active tryptase allele count is determined by sequencing the TPSAB1 and TPSB2 loci of the patient's genome.

29. The method of claim 28, wherein the sequencing is Sanger sequencing or massively parallel sequencing.

30. The method of claim 28 or 29, wherein the TPSAB1 locus is sequenced by a method comprising (i) amplifying a nucleic acid from the subject in the presence of a first forward primer comprising the nucleotide sequence of 5′-CTG GTG TGC AAG GTG AAT GG-3′ (SEQ ID NO: 31) and a first reverse primer comprising the nucleotide sequence of 5-AGG TOO AGO ACT CAG GAG GA-3′ (SEQ ID NO: 32) to form a TPSAB1 amplicon, and (ii) sequencing the TPSAB1 amplicon.

31. The method of claim 30, wherein sequencing the TPSAB1 amplicon comprises using the first forward primer and the first reverse primer.

32. The method of any one of claims 28-31, wherein the TPSB2 locus is sequenced by a method comprising (i) amplifying a nucleic acid from the subject in the presence of a second forward primer comprising the nucleotide sequence of 5′-GCA GGT GAG COT GAG AGT CC-3′ (SEQ ID NO: 33) and a second reverse primer comprising the nucleotide sequence of 5′-GGG ACC TTC ACC TGC TTC AG-3′ (SEQ ID NO: 34) to form a TPSB2 amplicon, and (ii) sequencing the TPSB2 amplicon.

33. The method of claim 32, wherein sequencing the TPSB2 amplicon comprises using the second forward primer and a sequencing reverse primer comprising the nucleotide sequence of 5′-CAG CCA GTG ACC CAG CAC-3′ (SEQ ID NO: 35).

34. The method of any one of claim 1, 2, 4-10, 12-15, 17-21, or 28-33, wherein the active tryptase allele count is determined by the formula: 4—the sum of the number of tryptase α and tryptase βIII frame-shift (βIII^{FS}) alleles in the patient's genotype.

35. The method of claim 34, wherein tryptase alpha is detected by detecting the c733 G>A SNP at TPSAB1 comprising the nucleotide sequence CTGCAGGCGGGCGTGGTCAGCTGGG[G/A]CGAGGGCTGTGCCCAGCCCAACCGG (SEQ ID NO: 36), wherein the presence of an A at the c733 G>A SNP indicates tryptase alpha.

36. The method of claim 34 or 35, wherein tryptase beta III^{FS} is detected by detecting a c980_981insC mutation at TPSB2 comprising the nucleotide sequence CACACGGTCACCCTGCCCCCTGCCTCAGAGACCTTCCCCCCC (SEQ ID NO: 37).

37. The method of any one of claim 1, 2, 4-10, 12-15, 17-21, or 28-36, wherein the reference active tryptase allele count is determined in a group of patients having the mast cell-mediated inflammatory disease.

38. The method of any one of claim 1, 2, 4-10, 12-15, 17-21, or 28-37, wherein the reference active tryptase allele count is 3.

39. The method of any one of claim 1, 2, 4-8, 15, 18-21 or 28-38, wherein the patient has an active tryptase allele count of 3 or 4.

40. The method of any one of claim 9, 10, 12, 15, 18-21, or 28-38, wherein the patient has an active tryptase allele count of 0, 1, or 2.

41. The method of any one of claim 1, 3-9, 11-14, or 16-27, wherein the tryptase is tryptase beta I, tryptase beta II, tryptase beta III, tryptase alpha I, or a combination thereof.

42. The method of any one of claim 1, 3-9, 11-14, 16-27, or 41, wherein the expression level of tryptase is a protein expression level.

43. The method of claim 42, wherein the protein expression level of tryptase is an expression level of active tryptase.

44. The method of claim 42, wherein the protein expression level of tryptase is an expression level of total tryptase.

45. The method of any one of claims 42-44, wherein the protein expression level is measured using an immunoassay, enzyme-linked immunosorbent assay (ELISA), Western blot, or mass spectrometry.

46. The method of any one of claim 1, 3-9, 11-14, 16-27, or 41, wherein the expression level of the tryptase is an mRNA expression level.

47. The method of claim 46, wherein the mRNA expression level is measured using a polymerase chain reaction (PCR) method or a microarray chip.

48. The method of claim 47, wherein the PCR method is qPCR.

49. The method of any one of claim 1, 3-9, 11-14, 16-27, or 41-48, wherein the reference level of tryptase is a level determined in a group of individuals having the mast cell-mediated inflammatory disease.

50. The method of claim 49, wherein the reference level of tryptase is a median level.

51. The method of any one of claims 1-50, wherein the sample from the patient is selected from the group consisting of a blood sample, a tissue sample, a sputum sample, a bronchiolar lavage sample, a mucosal lining fluid (MLF) sample, a bronchosorption sample, and a nasosorption sample.

52. The method of claim 51, wherein the blood sample is a whole blood sample, a serum sample, a plasma sample, or a combination thereof.

53. The method of claim 52, wherein the blood sample is a serum sample or a plasma sample.

54. The method of any one of claim 1-8 or 15-53, wherein the agent is a tryptase antagonist.

55. The method of claim 54, wherein the tryptase antagonist is a tryptase alpha antagonist or a tryptase beta antagonist.

56. The method of claim 55, wherein the tryptase antagonist is a tryptase beta antagonist.

57. The method of claim 55 or 56, wherein the tryptase beta antagonist is an anti-tryptase beta antibody or an antigen-binding fragment thereof.

58. The method of claim 57, wherein the antibody comprises the following six hypervariable regions (HVRs):
(a) an HVR-H1 comprising the amino acid sequence of DYGMV (SEQ ID NO: 1);
(b) an HVR-H2 comprising the amino acid sequence of FISSGSSTVYYADTMKG (SEQ ID NO: 2);
(c) an HVR-H3 comprising the amino acid sequence of RNYDDWYFDV (SEQ ID NO: 3);
(d) an HVR-L1 comprising the amino acid sequence of SASSSVTYMY (SEQ ID NO: 4);
(e) an HVR-L2 comprising the amino acid sequence of RTSDLAS (SEQ ID NO: 5); and
(f) an HVR-L3 comprising the amino acid sequence of QHYHSYPLT (SEQ ID NO: 6).

59. The method of claim 57 or 58, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

60. The method of claim 59, wherein the VH domain comprises the amino acid sequence of SEQ ID NO: 7.

61. The method of claim 59, wherein the VL domain comprises the amino acid sequence of SEQ ID NO: 8.

62. The method of claim 59, wherein the VH domain comprises the amino acid sequence of SEQ ID NO: 7 and the VL domain comprises the amino acid sequence of SEQ ID NO: 8.

63. The method of any one of claims 57-62, wherein the antibody comprises (a) a heavy chain comprising the amino acid sequence of SEQ ID NO: 9 and (b) a light chain comprising the amino acid sequence of SEQ ID NO: 10.

64. The method of any one of claims 57-62, wherein the antibody comprises (a) a heavy chain comprising the amino acid sequence of SEQ ID NO: 11 and (b) a light chain comprising the amino acid sequence of SEQ ID NO: 10.

65. The method of claim 57, wherein the antibody comprises the following six HVRs:
(a) an HVR-H1 comprising the amino acid sequence of GYAIT (SEQ ID NO: 12);
(b) an HVR-H2 comprising the amino acid sequence of GISSAATTFYSSWAKS (SEQ ID NO: 13);
(c) an HVR-H3 comprising the amino acid sequence of DPRGYGAALDRLDL (SEQ ID NO: 14);
(d) an HVR-L1 comprising the amino acid sequence of QSIKSVYNNRLG (SEQ ID NO: 15);
(e) an HVR-L2 comprising the amino acid sequence of ETSILTS (SEQ ID NO: 16); and
(f) an HVR-L3 comprising the amino acid sequence of AGGFDRSGDTT (SEQ ID NO: 17).

66. The method of claim 57 or 65, wherein the antibody comprises (a) a heavy chain variable (VH) domain comprising an amino acid sequence having at least 90%, at least 95%, or at least 99% sequence identity to the amino acid sequence of SEQ ID NO: 18; (b) a light chain variable (VL) domain comprising an amino acid sequence having at least 90%, at least 95%, or at least 99% identity to the amino acid sequence of SEQ ID NO: 19; or (c) a VH domain as in (a) and a VL domain as in (b).

67. The method of claim 66, wherein the VH domain comprises the amino acid sequence of SEQ ID NO: 18.

68. The method of claim 66, wherein the VL domain comprises the amino acid sequence of SEQ ID NO: 19.

69. The method of claim 66, wherein the VH domain comprises the amino acid sequence of SEQ ID NO: 18 and the VL domain comprises the amino acid sequence of SEQ ID NO: 19.

70. The method of any one of claim 57 or 65-69, wherein the antibody comprises (a) a heavy chain comprising the amino acid sequence of SEQ ID NO: 20 and (b) a light chain comprising the amino acid sequence of SEQ ID NO: 21.

71. The method of any one of claim 57 or 65-69, wherein the antibody comprises (a) a heavy chain comprising the amino acid sequence of SEQ ID NO: 22 and (b) a light chain comprising the amino acid sequence of SEQ ID NO: 21.

72. The method of any one of claims 54-71, wherein the therapy further comprises an IgE antagonist.

73. The method of any one of claim 9-21 or 28-53, wherein the agent is an FcεR antagonist.

74. The method of claim 73, wherein the FcεR antagonist is a Bruton's tyrosine kinase (BTK) inhibitor.

75. The method of claim 74, wherein the BTK inhibitor is GDC-0853, acalabrutinib, GS-4059, spebrutinib, BGB-3111, or HM71224.

76. The method of any one of claim 1-8 or 15-53, wherein the agent is an IgE⁺ B cell depleting antibody.

77. The method of claim 76, wherein the IgE⁺ B cell depleting antibody is an anti-M1′ domain antibody.

78. The method of any one of claim 1-8 or 15-53, wherein the agent is a mast cell or basophil depleting antibody.

79. The method of any one of claim 1-8 or 15-53, wherein the agent is a PAR2 antagonist.

80. The method of any one of claim 9-21 or 28-53, wherein the agent is an IgE antagonist.

81. The method of claim 72 or 80, wherein the IgE antagonist is an anti-IgE antibody.

82. The method of claim 81, wherein the anti-IgE antibody is an IgE blocking antibody and/or an IgE depleting antibody.

83. The method of claim 82, wherein the anti-IgE antibody comprises the following six HVRs:
(a) an HVR-H1 comprising the amino acid sequence of GYSWN (SEQ ID NO: 40);
(b) an HVR-H2 comprising the amino acid sequence of SITYDGSTNYNPSVKG (SEQ ID NO: 41);
(c) an HVR-H3 comprising the amino acid sequence of GSHYFGHWHFAV (SEQ ID NO: 42);
(d) an HVR-L1 comprising the amino acid sequence of RASQSVDYDGDSYMN (SEQ ID NO: 43);
(e) an HVR-L2 comprising the amino acid sequence of AASYLES (SEQ ID NO: 44); and
(f) an HVR-L3 comprising the amino acid sequence of QQSHEDPYT (SEQ ID NO: 45).

84. The method of claim 82 or 83, wherein the anti-IgE antibody comprises (a) a heavy chain variable (VH) domain comprising an amino acid sequence having at least 90%, at least 95%, or at least 99% sequence identity to the amino acid sequence of SEQ ID NO: 38; (b) a light chain variable (VL) domain comprising an amino acid sequence having at least 90%, at least 95%, or at least 99% identity to the amino acid sequence of SEQ ID NO: 39; or (c) a VH domain as in (a) and a VL domain as in (b).

85. The method of claim 84, wherein the VH domain comprises the amino acid sequence of SEQ ID NO: 38.

86. The method of claim 84, wherein the VL domain comprises the amino acid sequence of SEQ ID NO: 39.

87. The method of claim 84, wherein the VH domain comprises the amino acid sequence of SEQ ID NO: 38 and the VL domain comprises the amino acid sequence of SEQ ID NO: 39.

88. The method of any one of claims 81-87, wherein the anti-IgE antibody is omalizumab (XOLAIR®) or XmAb7195.

89. The method of claim 88, wherein the anti-IgE antibody is omalizumab (XOLAIR®).

90. The method of any one of claim 5-8, 13, 14, 18-21, or 28-89, wherein the Type 2 biomarker is a T_{H}2 cell-related cytokine, periostin, eosinophil count, an eosinophil signature, FeNO, or IgE.

91. The method of claim 90, wherein the T_{H}2 cell-related cytokine is IL-13, IL-4, IL-9, or IL-5.

92. The method of any one of claim 5-8, 13, 14, 18-21, or 28-91, wherein the T_{H}2 pathway inhibitor inhibits interleukin-2-inducible T cell kinase (ITK), Bruton's tyrosine kinase (BTK), Janus kinase 1 (JAK1), GATA binding protein 3 (GATA3), IL-9, IL-5, IL-13, IL-4, IL-33, OX40L, TSLP, IL-25, IL-9 receptor, IL-5 receptor, IL-4 receptor alpha, IL-13 receptoralpha1, IL-13 receptoralpha2, OX40, TSLP-R, IL-7Ralpha, IL-17RB, ST2, CCR3, CCR4, CRTH2, Flap, Syk kinase; CCR4, TLR9, or GM-CSF.

93. The method of any one of claim 1, 4-9, 12-14, or 17-92, further comprising administering an additional therapeutic agent to the patient.

94. The method of claim 93, wherein the additional therapeutic agent is selected from the group consisting of a corticosteroid, an IL-33 axis binding antagonist, a TRPA1 antagonist, a bronchodilator or asthma symptom control medication, an immunomodulator, a tyrosine kinase inhibitor, and a phosphodiesterase inhibitor.

95. The method of claim 94, wherein the additional therapeutic agent is a corticosteroid.

96. The method of claim 94 or 95, wherein the corticosteroid is an inhaled corticosteroid.

97. The method of any one of claims 1-96, wherein the mast cell-mediated inflammatory disease is selected from the group consisting of asthma, atopic dermatitis, chronic spontaneous urticaria (CSU), systemic anaphylaxis, mastocytosis, chronic obstructive pulmonary disease (COPD), idiopathic pulmonary fibrosis (IPF), and eosinophilic esophagitis.

98. The method of claim 97, wherein the mast cell-mediated inflammatory disease is asthma.

99. The method of claim 98, wherein the asthma is moderate to severe asthma.

100. The method of any one of claims 97-99, wherein the asthma is uncontrolled on a corticosteroid.

101. The method of any one of claims 97-100, wherein the asthma is T_{H}2 high asthma or T_{H}2 low asthma.

102. A kit for identifying a patient having a mast cell-mediated inflammatory disease who is likely to respond to a therapy comprising an agent selected from the group consisting of a tryptase antagonist, an IgE+ B cell depleting antibody, a mast cell or basophil depleting antibody, a protease activated receptor 2 (PAR2) antagonist, and a combination thereof, the kit comprising:
(a) reagents for determining the patient's active tryptase allele count or for determining the expression level of tryptase in a sample from the patient; and, optionally,
(b) instructions for using the reagents to identify a patient having a mast cell-mediated inflammatory disease who is likely to respond to a therapy comprising an agent selected from the group consisting of a tryptase antagonist, an IgE+ B cell depleting antibody, a mast cell or basophil depleting antibody, a PAR2 antagonist, and a combination thereof.

103. The kit of claim 102, wherein the agent is a tryptase antagonist, and the therapy further comprises an IgE antagonist.

104. A kit for identifying a patient having a mast cell-mediated inflammatory disease who is likely to respond to a therapy comprising an IgE antagonist or an FcεR antagonist, the kit comprising:
(a) reagents for determining the patient's active tryptase allele count or for determining the expression level of tryptase in a sample from the patient; and, optionally,
(b) instructions for using the reagents to identify a patient having a mast cell-mediated inflammatory disease who is likely to respond to a therapy comprising an IgE antagonist or an FcεR antagonist.

105. The kit of any one of claims 102-104, further comprising reagents for determining the level of a Type 2 biomarker in a sample from the patient.

106. An agent selected from the group consisting of a tryptase antagonist, an IgE+ B cell depleting antibody, a mast cell or basophil depleting antibody, a protease activated receptor 2 (PAR2) antagonist, and a combination thereof for use in a method of treating a patient having a mast cell-mediated inflammatory disease, wherein
(i) the genotype of the patient has been determined to comprise an active tryptase allele count that is at or above a reference active tryptase allele count; or
(ii) a sample from the patient has been determined to have an expression level of tryptase that is at or above a reference level of tryptase.

107. The agent for use of claim 106, wherein the patient has been determined to have a level of a Type 2 biomarker in a sample from the patient that is below a reference level of the Type 2 biomarker, and the agent is for use as a monotherapy.

108. The agent for use of claim 106, wherein the patient has been identified as having a level of a Type 2 biomarker in a sample from the patient that is at or above a reference level of the Type 2 biomarker, and the agent is for use in combination with a T_{H}2 pathway inhibitor.

109. An agent selected from an IgE antagonist or an FcεR antagonist for use in a method of treating a patient having a mast cell-mediated inflammatory disease, wherein
(i) the genotype of the patient has been determined to comprise an active tryptase allele count that is below a reference active tryptase allele count; or
(ii) a sample from the patient has been determined to have an expression level of tryptase that is below a reference level of tryptase.

110. The agent for use of claim 109, wherein the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.

111. The agent for use of any one of claims 106-110, wherein the active tryptase allele count is determined by sequencing the TPSAB1 and TPSB2 loci of the patient's genome.

112. The agent for use of claim 111, wherein the sequencing is Sanger sequencing or massively parallel sequencing.

113. The agent for use of claim 111 or 112, wherein the TPSAB1 locus is sequenced by a method comprising (i) amplifying a nucleic acid from the subject in the presence of a first forward primer comprising the nucleotide sequence of 5′-CTG GTG TOO AAG GTG AAT GG-3′ (SEQ ID NO: 31) and a first reverse primer comprising the nucleotide sequence of 5′-AGG TCC AGO ACT CAG GAG GA-3′ (SEQ ID NO: 32) to form a TPSAB1 amplicon, and (ii) sequencing the TPSAB1 amplicon.

114. The agent for use of claim 113, wherein sequencing the TPSAB1 amplicon comprises using the first forward primer and the first reverse primer.

115. The agent for use of any one of claims 111-114, wherein the TPSB2 locus is sequenced by a method comprising (i) amplifying a nucleic acid from the subject in the presence of a second forward primer comprising the nucleotide sequence of 5′-GCA GGT GAG COT GAG AGT CC-3′ (SEQ ID NO: 33) and a second reverse primer comprising the nucleotide sequence of 5′-GGG ACC TTC ACC TGC TTC AG-3′ (SEQ ID NO: 34) to form a TPSB2 amplicon, and (H) sequencing the TPSB2 amplicon.

116. The agent for use of claim 115, wherein sequencing the TPSB2 amplicon comprises using the second forward primer and a sequencing reverse primer comprising the nucleotide sequence of 5′-CAG CCA GTG ACC CAG CAC-3′ (SEQ ID NO: 35).

117. The agent for use of any one of claims 106-116, wherein the active tryptase allele count is determined by the formula: 4—the sum of the number of tryptase α and tryptase βIII frame-shift (βIII^{FS}) alleles in the patient's genotype.

118. The agent for use of claim 117, wherein tryptase alpha is detected by detecting the c733 G>A SNP at TPSAB1 comprising the nucleotide sequence CTGCAGGCGGGCGTGGTCAGCTGGG[G/A]CGAGGGCTGTGCCCAGCCCAACCGG (SEQ ID NO: 36), wherein the presence of an A at the c733 G>A SNP indicates tryptase alpha.

119. The agent for use of claim 117 or 118, wherein tryptase beta III^{FS} is detected by detecting a c980_981insC mutation at TPSB2 comprising the nucleotide sequence CACACGGTCACCCTGCCCCCTGCCTCAGAGACCTTCCCCCCC (SEQ ID NO: 37).

120. The agent for use of any one of claims 106-119, wherein the reference active tryptase allele count is determined in a group of patients having the mast cell-mediated inflammatory disease.

121. The agent for use of any one of claims 106-120, wherein the reference active tryptase allele count is 3.

122. The agent for use of any one of claims 106-121, wherein the patient has an active tryptase allele count of 3 or 4.

123. The agent for use of any one of claims 106-121, wherein the patient has an active tryptase allele count of 0, 1, or 2.

124. The agent for use of any one of claims 106-123, wherein the tryptase is tryptase beta I, tryptase beta II, tryptase beta III, tryptase alpha I, or a combination thereof.

125. The agent for use of any one of claims 106-124, wherein the expression level of tryptase is a protein expression level.

126. The agent for use of claim 125, wherein the protein expression level of tryptase is an expression level of active tryptase.

127. The agent for use of claim 125, wherein the protein expression level of tryptase is an expression level of total tryptase.

128. The agent for use of any one of claims 125-127, wherein the protein expression level is measured using an immunoassay, enzyme-linked immunosorbent assay (ELISA), Western blot, or mass spectrometry.

129. The agent for use of any one of claims 106-124, wherein the expression level of the tryptase is an mRNA expression level.

130. The agent for use of claim 129, wherein the mRNA expression level is measured using a polymerase chain reaction (PCR) method or a microarray chip.

131. The agent for use of claim 130, wherein the PCR method is qPCR.

132. The agent for use of any one of claims 106-131, wherein the reference level of tryptase is a level determined in a group of individuals having the mast cell-mediated inflammatory disease.

133. The agent for use of claim 132, wherein the reference level of tryptase is a median level.

134. The agent for use of any one of claims 106-133, wherein the sample from the patient is selected from the group consisting of a blood sample, a tissue sample, a sputum sample, a bronchiolar lavage sample, a mucosal lining fluid (MLF) sample, a bronchosorption sample, and a nasosorption sample.

135. The agent for use of claim 134, wherein the blood sample is a whole blood sample, a serum sample, a plasma sample, or a combination thereof.

136. The agent for use of claim 135, wherein the blood sample is a serum sample or a plasma sample.

137. The agent for use of any one of claim 106-108 or 111-136, wherein the agent is a tryptase antagonist.

138. The agent for use of claim 137, wherein the tryptase antagonist is a tryptase alpha antagonist or a tryptase beta antagonist.

139. The agent for use of claim 138, wherein the tryptase antagonist is a tryptase beta antagonist.

140. The agent for use of claim 138 or 139, wherein the tryptase beta antagonist is an anti-tryptase beta antibody or an antigen-binding fragment thereof.

141. The agent for use of claim 140, wherein the antibody comprises the following six hypervariable regions (HVRs):
(a) an HVR-H1 comprising the amino acid sequence of DYGMV (SEQ ID NO: 1);
(b) an HVR-H2 comprising the amino acid sequence of FISSGSSTVYYADTMKG (SEQ ID NO: 2);
(c) an HVR-H3 comprising the amino acid sequence of RNYDDWYFDV (SEQ ID NO: 3);
(d) an HVR-L1 comprising the amino acid sequence of SASSSVTYMY (SEQ ID NO: 4);
(e) an HVR-L2 comprising the amino acid sequence of RTSDLAS (SEQ ID NO: 5); and
(f) an HVR-L3 comprising the amino acid sequence of QHYHSYPLT (SEQ ID NO: 6).

142. The agent for use of claim 140 or 141, wherein the antibody comprises (a) a heavy chain variable (VH) domain comprising an amino acid sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).

143. The agent for use of claim 142, wherein the VH domain comprises the amino acid sequence of SEQ ID NO: 7.

144. The agent for use of claim 142, wherein the VL domain comprises the amino acid sequence of SEQ ID NO: 8.

145. The agent for use of claim 142, wherein the VH domain comprises the amino acid sequence of SEQ ID NO: 7 and the VL domain comprises the amino acid sequence of SEQ ID NO: 8.

146. The agent for use of any one of claims 140-145, wherein the antibody comprises (a) a heavy chain comprising the amino acid sequence of SEQ ID NO: 9 and (b) a light chain comprising the amino acid sequence of SEQ ID NO: 10.

147. The agent for use of any one of claims 140-145, wherein the antibody comprises (a) a heavy chain comprising the amino acid sequence of SEQ ID NO: 11 and (b) a light chain comprising the amino acid sequence of SEQ ID NO: 10.

148. The agent for use of claim 140, wherein the antibody comprises the following six HVRs:
(a) an HVR-H1 comprising the amino acid sequence of GYAIT (SEQ ID NO: 12);
(b) an HVR-H2 comprising the amino acid sequence of GISSAATTFYSSWAKS (SEQ ID NO: 13);
(c) an HVR-H3 comprising the amino acid sequence of DPRGYGAALDRLDL (SEQ ID NO: 14);
(d) an HVR-L1 comprising the amino acid sequence of QSIKSVYNNRLG (SEQ ID NO: 15);
(e) an HVR-L2 comprising the amino acid sequence of ETSILTS (SEQ ID NO: 16); and
(f) an HVR-L3 comprising the amino acid sequence of AGGFDRSGDTT (SEQ ID NO: 17).

149. The agent for use of claim 140 or 148, wherein the antibody comprises (a) a heavy chain variable (VH) domain comprising an amino acid sequence having at least 90%, at least 95%, or at least 99% sequence identity to the amino acid sequence of SEQ ID NO: 18; (b) a light chain variable (VL) domain comprising an amino acid sequence having at least 90%, at least 95%, or at least 99% identity to the amino acid sequence of SEQ ID NO: 19; or (c) a VH domain as in (a) and a VL domain as in (b).

150. The agent for use of claim 149, wherein the VH domain comprises the amino acid sequence of SEQ ID NO: 18.

151. The agent for use of claim 149, wherein the VL domain comprises the amino acid sequence of SEQ ID NO: 19.

152. The agent for use of claim 149, wherein the VH domain comprises the amino acid sequence of SEQ ID NO: 18 and the VL domain comprises the amino acid sequence of SEQ ID NO: 19.

153. The agent for use of any one of claim 140 or 148-152, wherein the antibody comprises (a) a heavy chain comprising the amino acid sequence of SEQ ID NO: 20 and (b) a light chain comprising the amino acid sequence of SEQ ID NO: 21.

154. The agent for use of any one of claim 140 or 148-152, wherein the antibody comprises (a) a heavy chain comprising the amino acid sequence of SEQ ID NO: 22 and (b) a light chain comprising the amino acid sequence of SEQ ID NO: 21.

155. The agent for use of any one of claims 137-154, wherein the tryptase antagonist is to be administered in combination with an IgE antagonist.

156. The agent for use of any one of claims 109-136, wherein the agent is an FcεR antagonist.

157. The agent for use of claim 156, wherein the FcεR antagonist is a Bruton's tyrosine kinase (BTK) inhibitor.

158. The agent for use of claim 157, wherein the BTK inhibitor is GDC-0853, acalabrutinib, GS-4059, spebrutinib, BGB-3111, or HM71224.

159. The agent for use of any one of claim 106-108 or 111-136, wherein the agent is an IgE⁺ B cell depleting antibody.

160. The agent for use of claim 159, wherein the IgE⁺ B cell depleting antibody is an anti-M1′ domain antibody.

161. The agent for use of any one of claim 106-108 or 111-136, wherein the agent is a mast cell or basophil depleting antibody.

162. The agent for use of any one of claim 106-108 or 111-136, wherein the agent is a PAR2 antagonist.

163. The agent for use of any one of claims 109-136, wherein the agent is an IgE antagonist.

164. The agent for use of claim 155 or 163, wherein the IgE antagonist is an anti-IgE antibody.

165. The agent for use of claim 164, wherein the anti-IgE antibody is an IgE blocking antibody and/or an IgE depleting antibody.

166. The agent for use of claim 165, wherein the anti-IgE antibody comprises the following six HVRs:
(a) an HVR-H1 comprising the amino acid sequence of GYSWN (SEQ ID NO: 40);
(b) an HVR-H2 comprising the amino acid sequence of SITYDGSTNYNPSVKG (SEQ ID NO: 41);
(c) an HVR-H3 comprising the amino acid sequence of GSHYFGHWHFAV (SEQ ID NO: 42);
(d) an HVR-L1 comprising the amino acid sequence of RASQSVDYDGDSYMN (SEQ ID NO: 43);
(e) an HVR-L2 comprising the amino acid sequence of AASYLES (SEQ ID NO: 44); and
(f) an HVR-L3 comprising the amino acid sequence of QQSHEDPYT (SEQ ID NO: 45).

167. The agent for use of claim 165 or 166, wherein the anti-IgE antibody comprises (a) a heavy chain variable (VH) domain comprising an amino acid sequence having at least 90%, at least 95%, or at least 99% sequence identity to the amino acid sequence of SEQ ID NO: 38; (b) a light chain variable (VL) domain comprising an amino acid sequence having at least 90%, at least 95%, or at least 99% identity to the amino acid sequence of SEQ ID NO: 39; or (c) a VH domain as in (a) and a VL domain as in (b).

168. The agent for use of claim 167, wherein the VH domain comprises the amino acid sequence of SEQ ID NO: 38.

169. The agent for use of claim 167, wherein the VL domain comprises the amino acid sequence of SEQ ID NO: 39.

170. The agent for use of claim 167, wherein the VH domain comprises the amino acid sequence of SEQ ID NO: 38 and the VL domain comprises the amino acid sequence of SEQ ID NO: 39.

171. The agent for use of any one of claims 164-170, wherein the anti-IgE antibody is omalizumab (XOLAIR®) or XmAb7195.

172. The agent for use of claim 171, wherein the anti-IgE antibody is omalizumab (XOLAIR®).

173. The agent for use of any one of claim 107, 108, or 110-172, wherein the Type 2 biomarker is a T_{H}2 cell-related cytokine, periostin, eosinophil count, an eosinophil signature, FeNO, or IgE.

174. The agent for use of claim 173, wherein the T_{H}2 cell-related cytokine is IL-13, IL-4, IL-9, or IL-5.

175. The agent for use of any one of claim 107, 108, or 110-174, wherein the T_{H}2 pathway inhibitor inhibits interleukin-2-inducible T cell kinase (ITK), Bruton's tyrosine kinase (BTK), Janus kinase 1 (JAK1), GATA binding protein 3 (GATA3), IL-9, IL-5, IL-13, IL-4, IL-33, OX40L, TSLP, IL-25, IL-9 receptor, IL-5 receptor, IL-4 receptor alpha, IL-13 receptoralpha1, IL-13 receptoralpha2, OX40, TSLP-R, IL-7Ralpha, IL-17RB, ST2, CCR3, CCR4, CRTH2, Flap, Syk kinase; CCR4, TLR9, or GM-CSF.

176. The agent for use of any one of claims 106-175, wherein the agent or combination is formulated for administration with an additional therapeutic agent.

177. The agent for use of claim 176, wherein the additional therapeutic agent is selected from the group consisting of a corticosteroid, an IL-33 axis binding antagonist, a TRPA1 antagonist, a bronchodilator or asthma symptom control medication, an immunomodulator, a tyrosine kinase inhibitor, and a phosphodiesterase inhibitor.

178. The agent for use of claim 177, wherein the additional therapeutic agent is a corticosteroid.

179. The agent for use of claim 177 or 178, wherein the corticosteroid is an inhaled corticosteroid.

180. The agent for use of any one of claims 106-179, wherein the mast cell-mediated inflammatory disease is selected from the group consisting of asthma, atopic dermatitis, chronic spontaneous urticaria (CSU), systemic anaphylaxis, mastocytosis, chronic obstructive pulmonary disease (COPD), idiopathic pulmonary fibrosis (IPF), and eosinophilic esophagitis.

181. The agent for use of claim 180, wherein the mast cell-mediated inflammatory disease is asthma.

182. The agent for use of claim 181, wherein the asthma is moderate to severe asthma.

183. The agent for use of any one of claims 180-182, wherein the asthma is uncontrolled on a corticosteroid.

184. The agent for use of any one of claims 180-183, wherein the asthma is T_{H}2 high asthma or T_{H}2 low asthma.

185. Use of an agent selected from the group consisting of a tryptase antagonist, an IgE+ B cell depleting antibody, a mast cell or basophil depleting antibody, a protease activated receptor 2 (PAR2) antagonist, and a combination thereof in the manufacture of a medicament for treating a patient having a mast cell-mediated inflammatory disease, wherein
(i) the genotype of the patient has been determined to comprise an active tryptase allele count that is at or above a reference active tryptase allele count; or
(ii) a sample from the patient has been determined to have an expression level of tryptase that is at or above a reference level of tryptase.

186. The use of claim 185, wherein the agent is a tryptase antagonist, and the medicament is formulated for administration with an IgE antagonist.

187. The use of claim 185 or 186, wherein the patient has been determined to have a level of a Type 2 biomarker in a sample from the patient that is below a reference level of the Type 2 biomarker, and the agent is for use as a monotherapy.

188. The use of claim 185 or 186, wherein the patient has been identified as having a level of a Type 2 biomarker in a sample from the patient that is at or above a reference level of the Type 2 biomarker, and the agent is for use in combination with a T_{H}2 pathway inhibitor.

189. Use of an IgE antagonist or an FcεR antagonist in the manufacture of a medicament for treating a patient having a mast cell-mediated inflammatory disease, wherein
(i) the genotype of the patient has been determined to comprise an active tryptase allele count that is below a reference active tryptase allele count; or
(ii) a sample from the patient has been determined to have an expression level of tryptase that is below a reference level of tryptase.

190. The use of claim 189, wherein the patient has been determined to have a level of a Type 2 biomarker in a sample from the patient that is at or above a reference level of the Type 2 biomarker, and the IgE antagonist or FcεR antagonist is for use in combination with an additional T_{H}2 pathway inhibitor.
